SEQUENCE LISTING (105 sequences)

SEQ ID NO: 1 - DNA, 2820 bp, Glycine max
  misc_feature (1)..(2820): Clone sde4c.pk0003.c8 encoding LOX1
ctttttcttcttcttcttcttctttattttctcattttgccaaactaaaatagttgtgttggtagctttggcaaagatgttttcagcaggccataagatcaaagggacagtggtgttgatgcccaagaatgagttggaagttaaccctgatggctcagcagttgacaaccttaatgctttcttgggccgtagtgtctcccttcagctcattagtgctaccaaggctgatgcacatggaaaaggaaaagttggaaaggatacgttcttggaagggattaatacttcgttaccaactttgggagcaggagagtctgcattcaatattcattttgaatgggacggaagcatgggaatccccggtgcgttttacataaagaactacatgcaagtttagttcttcctcaagagtttgactcttgaagccatttcaaaccaaggaaccatccgctttgtttgcaactcatgggtttataacactaaactttacaaaagcgtgcgcattttctttgccaaccatacatatgtacctagtgagacaccagcaccacttgtggagtacagagaagaagaattgaagagtttaagaggaaatggaacgggagagcgcaaggaatatgataggatctatgattatgatgtctacaatgatttgggcaacccagataagagtgaaaagttagctcgtccagttcttggagggtctagcacctttccctaccctcgtaggggaagaactggtcgaggtccaacagttacagacccgaatactgagaaacaaggcgaagtattttatgttccaagagatgaaaatttaggtcacttgaagtctaaggatgctcttgagattggaacaaaatctttatctcaaattgtccagccggcgttcgaatctgcgttcgatttgaaatccacgccaattgagtttcatagcttccaagatgtgcatgacttgtatgaaggtggaattaagcttcctagagatgtaattagcacaattatccccttaccagtgatcaaagaactctatcgtaccgatggtcaacacatcctcaagtttccacaacctcacgtcgttcaagtgagtcagtctgcatggatgactgatgaagaatttgctagagagatgattgctggtgtaaatccctgcgtaattcgtggtcttgaagagttccctccaaaaagtaatctggatcctgcaatctatggtgatcaaagcagtaagataacagcagattcccttgatctagatgggtacacaatggacgaggcacttggtagtcgaaggttatttatgttagattaccatgatatcttcatgccatatgtgaggcagataaatcagctgaattctgccaagacttatgcgacaaggactatcctttttttgagagaagatggaactttaaagccagtggccatcgaattaagtttgccacattctgctggggatctgtcagctgccgtcagtcaagtcgtcttacctgctaaggaaggtgttgagagcacaatttggctactagccaaagcttatgtcatcgtaaatgactcttgctaccatcaactcatgagccattggttaaatactcatgcggcgatggagccattcgtcatagcaacacaccgacatcttagcgtgcttcacccaatttacaagcttctgactcctcactatcgtaacaacatgaacatcaacgcacttgccaggcaatctctaattaatgctaatggcataatagagacaacctttttgccctcaaagtattctgtggagatgtcttcggcggtttataagaattgggttttcactgatcaagcactacctgcagatcttatcaagagaggagtggcaattaaggatccatcaaccccacatggagttcgtcttctgatagaggactatccttatgccgctgatggactggagatatgggctgcaattaagacatgggttcaagaatatgtgcccttgtactatgcaagagatgatgatgtcaaaaatgattctgaactccaacattggtggaaagaagctgtagagaaaggccatggtgatttgaaagacaagccatggtggcctaagttgcagacacttgaagaccttgttgaagtttgcctcattatcatatggattgcttcagctctccatgcagccgttaattttggtcagtatccttatggaggtttgataatgaaccgcccaactgcttctagaaggttgcttcctgagaaaggcaccccagaatatgaagaaatgattaataaccatgaaaaggcttatttgagaacaattacatcaaagttgccgactctcattagcctttcagtgatagagatcttgtcgacacatgcttctgatgaggtctaccttggccagagggacaacccacattggacctctgattcaaaagcattacaagcctttcaaaagtttggaaacaagctcaaggaaattgaggaaaaacttgtgaggaggaacaatgatccgagtctgcagggcaatcgacttggcccggttcaactgccatacactttgctttatcctagcagtgaggaagggttaacttttaggggaattccaaatagcatctctatctaagggagcctgtggtttactctaattactctatatatagtgcatgtgtaccttccaataaaaaagatgcaagactagagatccgataaatcttgcatcttatctaatgtttcaattatcttgtgttttaattaatgttgtaattgagctcacagtttggttgtggtttgcaaataataagagcagtgagcactattagtttgtgtttaaaaaaaaaaaaaaaaaaa

SEQ ID NO: 2 - DNA, 2869 bp, Glycine max
  misc_feature (1)..(2869): Clone se4.pk0007.e7 encoding LOX2
cttgttctttaattttctgattttagcaaattaaaatagtagtgttattggtaggttttggcaaagatgttttcagttccaggggtgtcgggaatcctgaacagaggaggagggcataagataaaagggacagtggtgttgatgaggaagaatgtgttggacttcaacagcgtggctgatcttactaaaggaaatgttgggggactcataggcaccggcctcaacgttgttggctcaacacttgataacctcactgctttcttgggccgaagtgtcgccctacagctcattagtgctaccaaacctcttgcaaatggaaaaggaaaagttggaaaggatacgttcttggaagggattattgtgtcgttaccaactttgggagcaggggagtctgcattcaatattcagtttgaatgggacgaaagcatgggaatccccggtgcgttttacataaagaactacatgcaagttgagttttacctcaagagtttgactcttgaacacgttccaaaccaaggaaccattcgctttgtttgcaactcatgggtttataacactaaactttacaaaagcgtgcgcattttctttgccaaccatacatatgttccaagtgagacaccagcagcacttgtggggtacagagaagaagaattgaagaatttaagaggagatggaaaaggagagcgcaaagaacatgataggatctatgattatgatgtctacaatgatttgggcaatccagatcacggtgaaaattttgctcgcccaattcttggagggtctagcactcatccctaccctcgtaggggaagaactggtcgatatccaacaaggaaagatcagaattctgagaagccaggcgaagtttatgttccaagagatgaaaattttggtcacttgaagtcctcggattttcttgcatatggaataaaatctttatctcaatatgtcttaccggcgttcgaatctgttttcgatttgaacttcacgccaaatgagtttgatagctcccaagatgtgcgtgacctccacgaaggtggaattaagcttcctacagaagtaattagcacaattatgcccttaccggtggtcaaagaactctttcgtacagatggtgaacaagtcctcaagtttccaccacctcatgtcattcaagtgagtaagtctgcatggatgaccgatgaagaatttgcaagagagatggttgcgggtgtaaatccatgcgtaattcgtggtcttcaagagtttcctccaaaaagcaatctggatcctacaatctatggtgagcaaaccagtaagataacagcagatgcccttgatctagatggatacacagtggacgaggcactagcaagtcggaggttatttatgttagattaccatgatgtattcatgccatatataaggcggataaatcagacatatgccaaggcttatgcgacaaggactatcctttttctgagagaaaatggaaccttaaagccagtggccatcgaattaagtttgccacatcctgctggggacctgtcaggtgctgtcagtcaagtcatcttacctgcaaaagaaggtgttgaaagtacaatttggctactggccaaagcttatgtggtcgtaaatgactcttgctatcatcaactcatgagccattggttaaatactcatgcggtgattgagccattcatcatagcaacaaaccgacaccttagtgctcttcacccaatttataagcttctaactcctcactaccgtgacaccatgaacatcaacgcacttgctaggcaatctctcattaatgctgatggcataatagagaaatcttttttgccctcaaagcattccgttgagatgtcttcagcggtttataagaattgggttttcactgatcaagcactacctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcttctgatgaggtctaccttggccaaagggacaacccacattggacctctgactcaaaagcattacaagcctttcaaaaatttggaaacaagctcaaagaaattgaggaaaaacttgcaaggaagaacaatgatcaaagtctctccaatcgacttgggccggttcaactgccatacactttgctccatcctaacagtgaggaagggttgacttgcagggggattcctaatagcatctctatctaagggagcatgtggtctactttaattacagtactgtacctaccttccaataaaaaagatgcaaggctagagatccaataaatcttgcatcctatctaatgtttcaattatcttgtgttttaatgctgtaatggagcctacaatttgcaaattaattaagagcaccgagcactattagtttgcgtttaaaaaaaaaaaaaaaaaa

SEQ ID NO: 3 - DNA, 2794 bp, Glycine max
  misc_feature (1)..(2794): Clone sgs1c.pk002.g4 encoding LOX3
ttggtgggttgcaaagatgcttgggggtcttctccataggggtcataagataaaagggacagtggtgttgatgcgcaagaatgtgttggacgtgaatagcgtaaccagcgttgggggaattattggtcaaggtctcgacttagttggctcaacactcgatactcttactgccttcttgggccgatccgtgtctctccagcttattagtgctaccaaagctgatgccaacggaaaagggaaacttggaaaggctacctttttggaaggtatcattacttcattgccaactttgggagcaggccaatctgcattcaaaattaattttgaatgggatgatgggagtggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatgaaggtgttgaaagttctatttggctgctagcaaaggcttatgtagttgtgaatgactcgtgctatcatcaacttgtcagccattggttaaacactcatgcagttgttgagccattcatcatagcaacaaacaggcatctcagtgttgttcaccctatttataaactccttcaccctcactatcgtgacaccatgaacataaatggccttgctcggttatcactggtcaacgacggtggcgttatagaacaaacatttttgtggggaaggtattctgtggaaatgtctgctgtagtttacaaggattgggtttttacagatcaagcattgcctgctgatcttataaaaagaggaatggcaattgaggatccatcgtgccctcatggcattcgccttgtgatagaggactacccttatgctgttgatggacttgagatatgggatgctatcaagacatgggtccatgaatacgttttcttgtactacaaatcagatgacacacttagagaagatcctgaactccaagcctgctggaaagaactcgtagaggtgggtcatggagacaagaaaaatgagccatggtggcctaagatgcaaactcgtgaagagctagttgaagcttgcgctatcatcatatggactgcttcagcacttcatgcagctgttaattttggacagtatccctatggaggtttaatcttaaaccgtccaactcttagtaggcgattcatgcctgagaaaggttctgctgagtatgaggagctgaggaagaatccccagaaggcttacttgaagactattacaccaaagtttcagacccttattgacctttctgttatagaaatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggaattcccaacagtatctctatctgaagaggtttgtggttccttaagttttgtggctttgaataaaagatagaagagaaggaatctgaatttcttcatccaacctgcacatggtaatctagtgtgtttgagtgctttcatgtttggctttaataaaataaaagcagttgttttgtaaaatgttcagaatcaataaaagcatttactgctttaaaaaaaaaaaaaaaaaaa

SEQ ID NO: 4 - DNA, 2808 bp, Glycine max
  misc_feature (1)..(2808): NCBI GI NO 18674 encoding LOX1
caggctttttctttttcttcttcttcttcttctttattttctcattttgccaaactaaaatagttgtgttggtagctttggcaaagatgttttcagcaggccataagatcaaagggacagtggtgttgatgcccaagaatgagttggaagttaaccctgatggctcagcagttgacaaccttaatgctttcttgggccgtagtgtctcccttcagctcattagtgctaccaaggctgatgcacatggaaaaggaaaagttggaaaggatacgttcttggaagggattaatacttcgttaccaactttgggagcaggagagtctgcattcaatattcattttgaatgggacggaagcatgggaatccccggtgcgttttacataaagaactacatgcaagttgagttcttcctcaagagtttgactcttgaagccatttcaaaccaaggaaccatccgctttgtttgcaactcatgggtttataacactaaactttacaaaagcgtgcgcattttctttgccaaccatacatatgtacctagtgagacaccagcaccacttgtgagttacagagaagaagaattgaagagtttaagaggaaatggaacgggagagcgcaaggaatatgataggatctatgattatgatgtctacaatgatttgggcaacccagataagagtgaaaagttagctcgtccagttcttggagggtctagcacctttccctaccctcgtaggggaagaactggtcgaggtccaacagttacagacccgaatactgagaaacaaggcgaagtattttatgttccaagagatgaaaatttaggtcacttgaagtctaaggatgctcttgagattggaacaaaatctttatctcaaattgtccagccggcgttcgaatctgcgttcgatttgaaatccacgccaattgagtttcatagcttccaagatgtgcatgacttgtatgaaggtggaattaagcttcctagagatgtaattagcacaattatccccttaccagtgatcaaagaactctatcgtaccgatggtcaacacatcctcaagtttccacaacctcacgtcgttcaagtgagtcagtctgcatggatgactgatgaagaatttgctagagagatgattgctggtgtaaatccctgcgtaattcgtggtcttgaagagttccctccaaaaagtaatctggatcctgcaatctatggtgatcaaagcagtaagataacagcagattcccttgatctagatgggtacacaatggacgaggcacttggtagtcgaaggttatttatgttagattaccatgatatcttcatgccatatgtgaggcagataaatcagctgaattctgccaagacttatgcgacaaggactatcctttttttgagagaagatggaactttaaagccagtggccatcgaattaagtttgccacattctgctggggatctgtcagctgccgtcagtcaagtcgtcttacctgctaaggaaggtgttgagagcacaatttggctactagccaaagcttatgtcatcgtaaatgactcttgctaccatcaactcatgagccattggttaaatactcatgcggcgatggagccattcgtcatagcaacacaccgacatcttagcgtgcttcacccaatttacaagcttctgactcctcactatcgtaacaacatgaacatcaacgcacttgccaggcaatctctaattaatgctaatggcataatagagacaacctttttgccctcaaagtattctgtggagatgtcttcggcggtttataagaattgggttttcactgatcaagcactacctgcagatcttatcaagagaggagtggcaattaaggatccatcaaccccacatggagttcgtcttctgatagaggactatccttatgccgctgatggactggagatatgggctgcaattaagacatgggttcaagaatatgtgcccttgtactatgcaagagatgatgatgtcaaaaatgattctgaactccaacattggtggaaagaagctgtagagaaaggccatggtgatttgaaagacaagccatggtggcctaagttgcagacacttgaagaccttgttgaagtttgcctcattatcatatggattgcttcagctctccatgcagccgttaattttggtcagtatccttatggaggtttgataatgaaccgcccaactgcttctagaaggttgcttcctgagaaaggcaccccagaatatgaagaaatgattaataaccatgaaaaggcttatttgagaacaattacatcaaagttgccgactctcattagcctttcagtgatagagatcttgtcgacacatgcttctgatgaggtctaccttggccagagggacaacccacattggacctctgattcaaaagcattacaagcctttcaaaagtttggaaacaagctcaaggaaattgaggaaaaacttgtgaggaggaacaatgatccgagtctgcagggcaatcgacttggcccggttcaactgccatacactttgctttatcctagcagtgaggaagggttaacttttaggggaattccaaatagcatctctatctaagggagcctgtggtttactctaattactctatatatagtgcatgtgtaccttccaataaaaaagatgcaagactagagatccgataaatcttgcatcttatctaatgtttcaattatcttgtgttttaattaatgttgtaattgagctcacagtttggttgtcaaaggtttgtaataagagcagtgagcactattagtttgtg

SEQ ID NO: 5 - DNA, 2766 bp, Glycine max
  misc_feature (1)..(2766): NCBI GI NO 170013 encoding LOX2
caagcttttctttttcttgttctttaattttctgattttagcaaattaaaatagtagtgttattggtaggttttggcaaagatgttttcagttccaggggtgtcgggaatcctgaacagaggaggagggcataagataaaagggacagtggtgttgatgaggaagaatgtgttggacttcaacagcgtggctgatcttactaaaggaaatgttgggggactcataggcaccggcctcaacgttgttggctcaacacttgataacctcactgctttcttgggccgaagtgtcgccctacagctcattagtgctaccaaacctcttgcaaatggaaaaggaaaagttggaaaggatacgttcttggaagggattattgtgtcgttaccaactttgggagcaggggagtctgcattcaatattcagtttgaatgggacgaaagcatgggaatccccggtgcgttttacataaagaactacatgcaagttgagttttacctcaagagtttgactcttgaagacgttccaaaccaaggaaccattcgctttgtttgcaactcatgggtttataacactaaactttacaaaagcgtgcgcattttctttgccaaccatacatatgttccaagtgagacaccagcagcacttgtggggtacagagaagaagaattgaagaatttaagaggagatggaaaaggagagcgcaaagaacatgataggatctatgattatgatgtctacaatgatttgggcaatccagatcacggtgaaaattttgctcgcccaattcttggagggtctagcactcatccctaccctcgtaggggaagaactggtcgatatccaacaaggaaagatcagaattctgagaagccaggcgaagtttatgttccaagagatgaaaattttggtcacttgaagtcctcggattttcttgcatatggaataaaatctttatctcaatatgtcttaccggcgttcgaatctgttttcgatttgaacttcacgccaaatgagtttgatagcttccaagatgtgcgtgacctccacgaaggtggaattaagcttcctacagaagtaattagcacaattatgcccttaccggtggtcaaagaactctttcgtacagatggtgaacaagtcctcaagtttccaccacctcatgtcattcaagtgagtaagtctgcatggatgaccgatgaagaatttgcaagagagatggttgcgggtgtaaatccatgcgtaattcgtggtcttcaagagtttcctccaaaaagcaatctggatcctacaatctatggtgagcaaaccagtaagataacagcagatgcccttgatctagatggatacacagtggacgaggcactagcaagtcggaggttatttatgttagattaccatgatgtattcatgccatatataaggcggataaatcagacatatgccaaggcttatgcgacaaggactatcctttttctgagagaaaatggaaccttaaagccagtggccatcgaattaagtttgccacatcctgctggggacctgtcaggtgctgtcagtcaagtcatcttacctgcaaaagaaggtgttgaaagtacaatttggctactggccaaagcttatgtggtcgtaaatgactcttgctatcatcaactcatgagccattggttaaatactcatgcggtgattgagccattcatcatagcaacaaaccgacaccttagtgctcttcacccaatttataagcttctaactcctcactaccgtgacaccatgaacatcaacgcacttgctaggcaatctctcattaatgctgatggcataatagagaaatcttttttgccctcaaagcattccgttgagatgtcttcagcggtttataagaattgggttttcactgatcaagcactacctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcttctgatgaggtctaccttggccaaagggacaacccacattggacctctgactcaaaagcattacaagcctttcaaaaatttggaaacaagctcaaagaaattgaggaaaaacttgcaaggaagaacaatgatcaaagtctctccaatcgacttgggccggttcaactgccatacactttgctccatcctaacagtgaggggttgacttgcagggggattcctaatagcatctctatctaagggagcatgtggtctactttaattacagtactgtacctaccttccaataaaaaagatgcaaggctagagatccaataaatcttgcac

SEQ ID NO: 6 - DNA, 2811 bp, Glycine max
  misc_feature (1)..(2811): NCBI GI NO 1794171 encoding LOX3
gtagtgttggtgggttgcaaagatgcttgggggtcttctccataggggtcataagataaaagggacagtggtgttgatgcgcaagaatgtgttggacgtgaatagcgtaaccagcgttgggggaattattggtcaaggtctcgacttagttgggtcaacactcgatactcttactgccttcttgggccgatccgtgtctctccagcttattagtgctaccaaagctgatgccaacggaaaagggaaacttggaaaggctacctttttggaaggtatcattacttcattgccaactttgggagcaggccaatctgcattcaaaattaattttgaatgggatgatgggagtggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatgaaggtgttgaaagttctatttggctgctagcaaaggcttatgtagttgtgaatgactcgtgctatcatcaacttgtcagccattggttaaacactcatgcagttgttgagccattcatcatagcaacaaacaggcatctcagtgttgttcaccctatttataaactccttcaccctcactatcgtgacaccatgaacataaatggccttgctcggttatcactggtcaacgacggtggcgttatagaacaaacatttttgtggggaaggtattctgtggaaatgtctgctgtagtttacaaggattgggtttttacagatcaagcattgcctgctgaccttataaaaagaggaatggcaattgaggatccatcgtgccctcatggcattcgccttgtgatagaggactacccttatactgttgatggacttgagatatgggatgctatcaagacatgggtccatgaatacgttttcttgtactacaaatcagatgacacacttagagaagatcctgaactccaagcctgctggaaagaactcgtagaggtgggtcatggagacaagaaaaatgagccatggtggcctaagatgcaaactcgtgaagagctagttgaagcttgcgctatcatcatatggactgcttcagcacttcatgcagctgttaattttggacagtatccctatggaggtttaatcttaaaccgtccaactcttagtaggcgattcatgcctgagaaaggttctgctgagtatgaggagctgaggaagaatccccagaaggcttacttgaagactattacaccaaagtttcagacccttattgacctttctgttatagaaatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggaattcccaacagtatctctatctgaagaggtttgtggttccttaagttttgtggatttgaataaaagatagaagagaaggaatctgaatttcttcatccaacctgcacatggtaatctagtgtgtttgagtgctttcatgtttggctttaataaaataaaagcagttgttttgtaaaatgttcagaatcaataaaagcatttactgctttgctttgtgcgctaaaaaaaaaaaaaaaaaa

SEQ ID NO: 7 - DNA, 32 bp, Artificial Sequence
  longest stretch of identical nucleotides shared by LOX1, LOX2, and  LOX3
aggatctatgattatgatgtctacaatgattt

SEQ ID NO: 8 - DNA, 50 bp, Artificial Sequence
  longest stretch of identical nucleotides shared by LOX1 and
cttctagaaggttgcttcctgagaaaggcaccccagaatatgaagaaatg

SEQ ID NO: 9 - DNA, 1425 bp, Glycine max
  misc_feature (1)..(1425): cDNA insert in clone sr1.pk0097.b11 encoding CHS
attctctctactactgctagctgtgacttgttttgagttcaatcaaatcgcattggtatctgaaatcaggaaagatggttagcgtagctgagatcaggcaggcacaaagggcagaaggcccagcaaccatccttgccattggaactgcaaacccaccaaaccgtgttgatcagagcacctatcctgattactacttcagaatcaccaacagtgaccacatgaccgagctcaaagagaaatttcagcgcatgtgtgacaagtctatgatcaagacgagatatatgtacctaaacgaagagatcttgaaagagaatccaaacatgtgtgcttacatggcaccttctttggatgctaggcaagacatggtggtggtagaggtaccaaagctagggaaagaggctgcagtaaaggccataaaggagtggggccagccaaagtcaaagattacccacttgatcttctgcaccactagcggtgtggacatgcctggtgctgattaccaactcaccaaacaattgggccttcgcccttatgtgaagaggtacatgatgtaccaacaaggttgctttgcaggtggcacggttcttcgtttggccaaggatttggctgagaacaacaagggtgcacgtgtgcttgttgtctgctctgagatcactgcagtcacattccgtgggccaagtgacactcaccttgatagtcttgtgggccaagcattgtttggagatggagctgctgcagtcattgttggttctgacccaattccacaagttgagaagcctttgtatgagcttgtttggactgcacaaacaattgctccagacagtgaaggtgctattgatggacaccttcgtgaagttggactcacatttcacctcctcaaggatgttcccgggattgtctcaaagaacattgataaggcactttttgaggctttcaacccattgaacatctctgattacaactccatcttttggattgcacaccctggtgggcctgcgattttagaccaagttgagcaaaagttgggtctcaaacctgagaagatgaaggccactagagatgtgcttagtgaatatgggaacatgtcaagtgcttgtgttcttttcatcttggatgagatgaggaggaaatctgctgaaaatggacataaaaccacaggtgaaggacttgaatggggtgtgttgttcggttttggacctggacttaccattgaaactgttgttttgcatagtgtggccatctgagatgcctcatatattatttcattattgtgtaccacttttcaaacttgcttggggtttgtaacaacaacaaccaccaaaaaaaaaaaaactcgtttagagtttgtttgtgtacgtttatattaaaataataatacgtgtttggtggctttttagcctaattatcaaaaaaaaaaaaaaaaaa

SEQ ID NO: 10 - DNA, 1654 bp, Glycine max
  misc_feature (1)..(1654): cDNA in clone sdp3c.pk017.j17 encoding HPL
gcacgagaaccaccacaccacacagcacttcccttctcctcctccaccatgtcattgccaccgccgtcgctggtaacggcggcgactccgacggagcttccgatccggcagattccgggaagctacgggttcccgttgctgggccccatctcggatcggcttgactacttctggttccagaagccagagagcttcttcagaaagcgagtggagaaatacaagagcaccgtgttccgcaccaatgttcctccctccttccccttcttcgttaacgttaatcccaacgtaatcgcggttctcgacgtgaaatcattctcccacctcttcgacatggacctcgtcgacaagaaggatgttctcgtcggagactttgtccccagcgtcgccttcaccggaaacatgagagtcggcgtttaccaggacactaccgaacctcaacattccaaggtgaagaactacatcatggatattctgaaaagaagctcaggcatatgggtatcagaactagaatcaaacctagacacactgtgggacaacatcgaagcatcactctcgaaatcctcatcagtttcgtaccttttcccgttacaacagtttctcttcacgttcctctgcaaagtccttgccggtgcagaccctgcacgtgaccctaaaatcgcagagtcaggctattccatgcttaatagttggctcgctcttcagctgctccccacagtcagcgtgggcatccttcaacccctggaagaaatcttcctccactcctttgcctaccctttcttcctcgtcggtggaaactacaacaacctctacaacttcatcaagcaacaaggcaaggacactataaaccggggcgcaatcgggttcgggttaaccgaagaagaagccatccacaatttgctcttcgtgctgggcttcaactcctacggcggattctccatttttctcccctctctgatcgatgccatagccagcaactccgcattgcaggagaagcttaaaaaagaagcgagagaaaagggcgggtcaacgctgaccttcgactcggtcaaagagatggacctgatccaatccgtggtctacgaaacgctgcgcatgaaccctccggttccactccagtacggtcgagcccgaaaggacttccggttgagttcgcacgactcggtcttccacgtcaagaagggcgagttgctgtgcggctttcagaagctcgtcatgagggactccgttatattcgacgaaccggaccggttcaagccggaccggttcacccaagagaaaggggcccagttgttgaactacttgtattggtccaatgggcctcagaccgggtcgcccagtgtgtccaataaacagtgcgcgggtaaggacgttgtcacactcaccgcggctttgattgtggcgtacctttttcgcaggtatgattccatccaaggggatggcagttccatcactgcccttcaaaagaccaagtgaaagaagggtaaatgtgtattttcattgtggagcttgtattttatctaaggtatattatttacttgagaatgatgatctatttatatagaagaggcaggtttatgcaactctttcataaactacaatcttcaattgtaactaacagatgactgatacattttgtaaaaaaaaaaaaaaaaaa

SEQ ID NO: 11 - protein, 474 aa, Glycine max
  MISC_FEATURE (1)..(474): translation of nucleotides 49-1473 of clone sdp3c.pk017.j17
MSLPPPSLVTAATPTELPIRQIPGSYGFPLLGPISDRLDYFWFQKPESFFRKRVEKYKSTVFRTNVPPSFPFFVNVNPNVIAVLDVKSFSHLFDMDLVDKKDVLVGDFVPSVAFTGNMRVGVYQDTTEPQHSKVKNYIMDILKRSSGIWVSELESNLDTLWDNIEASLSKSSSVSYLFPLQQFLFTFLCKVLAGADPARDPKIAESGYSMLNSWLALQLLPTVSVGILQPLEEIFLHSFAYPFFLVGGNYNNLYNFIKQQGKDTINRGAIGFGLTEEEAIHNLLFVLGFNSYGGFSIFLPSLIDAIASNSALQEKLKKEAREKGGSTLTFDSVKEMDLIQSVVYETLRMNPPVPLQYGRARKDFRLSSHDSVFHVKKGELLCGFQKLVMRDSVIFDEPDRFKPDRFTQEKGAQLLNYLYWSNGPQTGSPSVSNKQCAGKDVVTLTAALIVAYLFRRYDSIQGDGSSITALQKTK

SEQ ID NO: 12 - DNA, 1752 bp, Glycine max
  misc_feature (1)..(1752): cDNA insert in sdp4c.pk015.e22 clone encoding HPL
acaacactacactagcaagcacacacctttagcacaatcaccaatggcttcttccgacagcaagcttcccctgaaacccatccctggaagctacgggcttcctttctttggacccatgagtgacagacatgactatttctacaaccaaggacgcgacaagttctttgccgaacgaattaaaaagtacaactctacggttatacgaaccaacatgccaccgggtcctttcatttcctctaatcctagagtcatcgctctcctcgacggtgtctccttccccattctattcgacaactccaaggtcgataagcgcgatgttctcgacggcaccttcatgccttccacctccttcaccggcggttaccgcgcgtgtgccttccaggacaccaccgaaccctcccacgcgctcctcaagcgcttctaccttaacttcctcgcctccaagcacgaaaccttcctcccactcttccgcaacaacctctccgaccacttctctgatctggaagacaagctggccggcaaatccggcaaggccagcttcaactcctccgtcggctccgccaccttcaacttcctcttccgtctcctctccgacaaagacccctccgaaaccataatcggctccgacggccccagcctggtccaaacctggctggcagctcagctggctcctctggccactctaggcttacccaggatcttcaactacgtggaggatttcttaatcaggtcaatcccctttccagcatggactgtcaaatccagctacaagaaactctacgaaggtctctcgacagcaggtactgcgattctggacgaagcggagcgcgtggggataaagagggacgaagcgtgccacaatcttgtgttcatgttatcgttcaacgcgcagggtgggttagtgaaccagtttccgattttgatcaagtggctaggactggctggggagggtttacacaagcagctcgcggaggagatcaggaccgttgttaaggacgaaggaggggtgagtctccgggcgttggatcagatgactttgaccaaatcagtggtgtatgaggtcctgaggatagagcccgcggtgccgttccagtacgcgaaggccagggaggatctggtggtggagagccacgatgcggcgtacgagatcaagaagggagagatgatcttcggatatcagccgttcgctaccaaggatccgaagatcttcgagaacgccgaggactttgtggcccacaggttccttggccacgacggggaaaagctcttgagacacgtcttgtggtccaacggaccccagacggaggagcccacaccggatgataaacagtgtcccgccaagaatctggtggtgctcatgtgcaggctctacttggtggaattcttcctgcgttacgacacgtttacgttcgattttaaaccagttgttctgggtcccgatgttaccatcaagtcactcgccaaggcttcttccttctgatcctcctcatcacctactctacctcaaacttaaaatcaatcttctttttaatgtgtattagttaattaataattaaataaaacaaagtaaatatatatattaaaagtaggatgtgcactggacagcacagcagtactcatgaattgccgcttgttgcattttgtgagagaggtgtgtacattatttgctacctgtaacttttgcagagtgctctctatatatataatgttaaagttttacttgttttaaaaaaaaaaaaaaaaaaaaaaaaa

SEQ ID NO: 13 - protein, 478 aa, Glycine max
  MISC_FEATURE (1)..(478): translation of nucleotides 44-1480 of clone sdp4c.pk015.e22
MASSDSKLPLKPIPGSYGLPFFGPMSDRHDYFYNQGRDKFFAERIKKYNSTVIRTNMPPGPFISSNPRVIALLDGVSFPILFDNSKVDKRDVLDGTFMPSTSFTGGYRACAFQDTTEPSHALLKRFYLNFLASKHETFLPLFRNNLSDHFSDLEDKLAGKSGKASFNSSVGSATFNFLFRLLSDKDPSETIIGSDGPSLVQTWLAAQLAPLATLGLPRIFNYVEDFLIRSIPFPAWTVKSSYKKLYEGLSTAGTAILDEAERVGIKRDEACHNLVFMLSFNAQGGLVNQFPILIKWLGLAGEGLHKQLAEEIRTVVKDEGGVSLRALDQMTLTKSVVYEVLRIEPAVPFQYAKAREDLVVESHDAAYEIKKGEMIFGYQPFATKDPKIFENAEDFVAHRFLGHDGEKLLRHVLWSNGPQTEEPTPDDKQCPAKNLVVLMCRLYLVEFFLRYDTFTFDFKPVVLGPDVTIKSLAKASSF

SEQ ID NO: 14 - DNA, 1729 bp, Glycine max
  misc_feature (1)..(1729): cDNA insert in clone sgs4c.pk002.f8 encoding HPL
gcacgagcactcctcctcctccctctctcttccaaacacacatcctcaaccatggcagcaccatcttcagagacaaagtcaccatcatcctcgaacacgcagcttccgctgaaaccaatcccaggcagctacggaatgccgttttttggagcaataagcgacagacacaactacttctaccaccaaggacgcgacaagttcttcgcgacgaggattgaaaaacacaactccaccgtgatccgaaccaacatgcctccggggcccttcatctcctcggaccctcgtgtcgtcgcgttgttggacggtgcctccttcccgatcctcttcgacaacgacaaggtcgagaagctcaacgttctcgacggcaccttcatgccttccaccaagttcaccggcgggttccgcgtctgcgcctacctcgacaccaccgaacccaaccacgcactcatcaaacagttcttccttaacgtcctcgccaagcgaaaagactcttttgtccctctgttccgaaactgcctccaggagtcgttcgcggagattgaggaccagttgagtaaaaacaccaaagcggacttcaacactgtgttcagtgacgcttccttcaacttcatgttcaggttgttctgtgatggcaaagacccttcgcagaccaacctcggttctaagggaccgaagctcgtggacacatggcttctctttcagctggccccactggcaactctaggcctccccaaaatcttcaactacatcgaagacttcctaatccgcacactccctttccccgcgtgcctcacaaagtccggctacaagaacctctacgaggcctttaaaacgcacgcaacgacagcactcgacgaggccgaaaagctaggcctcaaacgaaacgaagcgtgccacaacgtcgttttcactgcagggttcaacgcctacggagggttaaagaaccagttcccctacgtcttaaaatggctagggctaagcggcgaaaagctccatgctgacctcgcgcgtgaggttcggcgcgtggtgaacgacgagggaggtgtcacgttcaccgcgttggagaacatgcctctcgtgaagtccgtggtgtacgaggtaatgcggatcgaacctgcggtgccgtaccagtacgcacgcgcgagggagaaccttgtcgtttcgagccatgacgcgtcgtttgaggtgaagaagggggagatgctgttcgggtaccagccgtttgcgacgagggatccgaggatcttcgaggatgctgaggtgtttgttccgcggaggtttgttggggagggggagaagatgctgaagcacgtgctgtggtcgaatgggagggagacggaggagccttcggcgagtaataagcagtgtcccgggaagaatctggtggtgctgctgtgcaggttgtttctggtggaactctttttgcgttatgatacgtttgagtttgagtatacgcaggctgggtttggtcctactattaccattaagtccctcactaaggcctctaccatctgagtttggggtttattatcttccttgtggcagcgtggttttagcttaatttttaaaaaaatgtatgtgtttttaactttttgtttttttctaaagacatgttgtaaaaaaaataagcgatgttttttttaaatttaatttggaccgaaaatattattattattgttatatattatatattaaaaacaattttatattaaaaaaaaaaaaaaaaaaa

SEQ ID NO: 15 - protein, 487 aa, Glycine max
  MISC_FEATURE (1)..(487): translation of nucleotides 52-1515 of cDNA insert in clone sgs4c.pk002.f8
MAAPSSETKSPSSSNTQLPLKPIPGSYGMPFFGAISDRHNYFYHQGRDKFFATRIEKHNSTVIRTNMPPGPFISSDPRVVALLDGASFPILFDNDKVEKLNVLDGTFMPSTKFTGGFRVCAYLDTTEPNHALIKQFFLNVLAKRKDSFVPLFRNCLQESFAEIEDQLSKNTKADFNTVFSDASFNFMFRLFCDGKDPSQTNLGSKGPKLVDTWLLFQLAPLATLGLPKIFNYIEDFLIRTLPFPACLTKSGYKNLYEAFKTHATTALDEAEKLGLKRNEACHNVVFTAGFNAYGGLKNQFPYVLKWLGLSGEKLHADLAREVRRVVNDEGGVTFTALENMPLVKSVVYEVMRIEPAVPYQYARARENLVVSSHDASFEVKKGEMLFGYQPFATRDPRIFEDAEVFVPRRFVGEGEKMLKHVLWSNGRETEEPSASNKQCPGKNLVVLLCRLFLVELFLRYDTFEFEYTQAGFGPTITIKSLTKASTI

SEQ ID NO: 16 - DNA, 1756 bp, Glycine max
  misc_feature (1)..(1756): cDNA insert in clone sgs1c.pk006.o20 encoding soybean IFS
gtaattaacctcactcaaactcgggatcacagaaaccaacaacagttcttgcactgaggtttcacgatgttgctggaacttgcacttggtttgtttgtgttagctttgtttctgcacttgcgtcccacaccaagtgcaaaatcaaaagcacttcgccacctcccaaaccctccaagcccaaagcctcgtcttcccttcattggccaccttcacctcttaaaagataaacttctccactatgcactcatcgatctctccaaaaagcatggccccttattctctctctccttcggctccatgccaaccgtcgttgcctccacccctgagttgttcaagctcttcctccaaacccacgaggcaacttccttcaacacaaggttccaaacctctgccataagacgcctcacttacgacaactctgtggccatggttccattcggaccttactggaagttcgtgaggaagctcatcatgaacgaccttctcaacgccaccaccgtcaacaagctcaggcctttgaggacccaacagatccgcaagttccttagggttatggcccaaagcgcagaggcccagaagccccttgacgtcaccgaggagcttctcaaatggaccaacagcaccatctccatgatgatgctcggcgaggctgaggagatcagagacatcgctcgcgaggttcttaagatcttcggcgaatacagcctcactgacttcatctggcctttgaagtatctcaaggttggaaagtatgagaagaggattgatgacatcttgaacaagttcgaccctgtcgttgaaagggtcatcaagaagcgccgtgagatcgtcagaaggagaaagaacggagaagttgttgagggcgaggccagcggcgtcttcctcgacactttgcttgaattcgctgaggacgagaccatggagatcaaaattaccaaggagcaaatcaagggccttgttgtcgactttttctctgcagggacagattccacagcggtggcaacagagtgggcattggcagagctcatcaacaatcccagggtgttgcaaaaggctcgtgaggaggtctacagtgttgtgggcaaagatagactcgttgacgaagttgacactcaaaaccttccttacattagggccattgtgaaggagacattccgaatgcacccaccactcccagtggtcaaaagaaagtgcacagaagagtgtgagattaatgggtatgtgatcccagagggagcattggttcttttcaatgtttggcaagtaggaagggaccccaaatactgggacagaccatcagaattccgtcccgagaggttcttagaaactggtgctgaaggggaagcagggcctcttgatcttaggggccagcatttccaactcctcccatttgggtctgggaggagaatgtgccctggtgtcaatttggctacttcaggaatggcaacacttcttgcatctcttatccaatgctttgacctgcaagtgctgggccctcaaggacaaatattgaaaggtgatgatgccaaagttagcatggaagagagagctggcctcacagttccaagggcacatagtctcgtttgtgttccacttgcaaggatcggcgttgcatctaaactcctttcttaattaagataatcatcatatacaatagtagtgtcttgccatcgcagttgctttttatgtattcataatcatcatttcaataaggtgtgactggtacttaatcaagtaattaaggttacatacatgc

SEQ ID NO: 17 - DNA, 1465 bp, Glycine max
  misc_feature (1)..(1465): cDNA insert in clone sfl1.pk0040.g11 encoding soybean F3H
gcacgagggcacgaggaagcattgcattctgctatttaattccactacgtacacgcacattctcctcaaagacaacaatggcaccaacagccaagactctgacttacctggcccaggagaaaaccctagaatcgagcttcgttcgggacgaggaggagcgtcccaaggttgcctacaacgaattcagcgacgagatcccagtgatttctcttgccggaatcgacgaggtggatggacgcagaagagagatttgtgagaagatcgtggaggcttgcgagaattggggtatattccaggttgttgatcacggtgtggatcaacaactcgtggccgagatgacccgtctcgccaaagagttctttgctttgccaccggacgagaagcttcgttttgatatgtccggcgccaaaaagggtggattcattgtctccagccatctccaaggggaatcggtgcaggactggagagaaatagtgacatacttttcgtacccaaaaagagagagggactattcaaggtggccagacacgccagaagggtggagatcggtgactgaggaatacagcgacaaagtaatgggtctagcttgcaagctcatggaggtgttgtccgaagcaatggggttagagaaagagggtttaagcaaagcatgtgttgacatggaccagaaggtggtggttaattactaccccaaatgccctcaacctgacctcactcttggcctgaagcgccacacggatccgggcactatcaccttgctgcttcaggaccaagtgggtggacttcaagccaccagggacaatggcaaaacatggatcaccgttcagcctgtggaggctgccttcgtcgtcaatcttggagatcatgctcattatctgagcaatggaaggttcaagaatgctgatcaccaagcggtggtgaactcaaaccatagccgtttgtccatagccacttttcaaaacccagcaccaaatgcaactgtttaccctctgaagataagagaaggagagaagcctgtgatggaggaaccaatcacttttgctgaaatgtacaggaggaagatgagcaaggacattgagattgcaaggatgaagaagctggctaaggaaaagcatttgcaggaccttgagaatgaaaagcatttgcaagaacttgatcagaaggcaaaacttgaggccaagcctttgaaggagattcttgcttaattaataataattacatatgtatcatttgcatgcccccttggtgtttttagtattttttaagggccatgaattaataatagtccttacctttgtgcttttgtacgtcttatgatttatcctttgtggggatatcatgtgttgtgttcagttgcctatgtcttattagctagctggctcatctatgtataccttatatgtgcctctattataaatgaaaataagtggcactgtctttattaaaaaaaaaaaaaaaaaaaaaa

SEQ ID NO: 18 - DNA, 2478 bp, Glycine max
  misc_feature (1)..(2478): cDNA in clone src3c.pk024.m11 encoding an b-amyrin synthase
ggtttgtttggtgtgagtgaatagggatcagggatgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcccttggttttttgtatgtatattacaggaaatcttgaatcagtatttccagaagaacatcgcaaagaaattcttcgttacacatattatcaccagaatgaagacggaggatggggactacacatagagggtcatagcactatgttttgtactgcactgaactatatatgcatgcgaatgcttggagaaggacctaatggaggtcatgacaatgcttgtgctagagcaagaaagtggattcgagatcatggtggtgtaacacatataccttcatggggaaaaacttggctttcgatactcggtgtatttgattggtgcggaagcaacccaatgcccccagagttttggatccttccatcttttcttcctatgcatccagctaagatgtggtgttactgtcgattggtatacatgcctatgtcttacttatatgggaagaggtttgtgggtccaatcacaccactcatcttacaattaagagaagagttgtttactcaaccttatgaaaaagttaattggaagaaagcgcgtcaccaatgtgcaaaggaagatctttactatccccatcctttgatacaagacctaatatgggatagtttatacatattcactgaaccgctacttactcgttggcctttcaacaagttgattagagaaaaggcccttcaagtaactatgaaacatattcattatgaagatgagactagtcgatacataaccattggttgtgtggaaaaggttttatgtatgcttgcttgttgggtggaagatccaaacggagatgctttcaagaagcatcttgcaagggtcccagattacttatgggtttctgaagatggaatgaccatgcagagttttggtagccaagaatgggatgctggctttgctgttcaagctttgcttgccactaacataattgaagaaattggtcctacgtttgcaaaaggacatgatttcatcaagaagtctcaggtgaaggataatccttttggagattttaaaagtatgcatcgtcatatttctaaagggtcttggacattctctgatcaagaccatggatggcaagtttctgattgcactgcagaaggtttaaagtgttgtctacttctatcaatgttgccaccagagattgtgggagaaaagatggaacctgaaagattatacgattcagtcaatgtcttgttgtcgcttcagagtaaaaaaggtggtttagcagcatgggagcctgcaggagctcaagagtggttagaattactcaatcccacagaattttttgcggacattgtagttgaacatgaatatgttgagtgcactggatctgcaatccaagctttagttttgttcaagaagctatatccaggacataggaagaaagagatagaaaatttcattaccaatgcagttcgattccttgaagatacacaaacagctgatggttcatggtatggaaattggggagtttgcttcacttatggctcttggtttgcacttggaggtctagcagctgctggtaagacttacaccaattgtgctgccattcgcaaagccgttaaatttctacttacaacacaaagagaggacggtggatggggagagagttatctttcaagcccaaaaaagatatatgtacctctagaaggaagccgatcaaatgttgtacatacagcatgggctcttatgggactaattcatgctggacaggcggatagagaccccatgcctcttcaccgtgctgcaaagttgctcattaattctcagttggaagagggtgattggccccaacaggaaatcacgggagtattcatgaaaaattgcatgttgcattatccaatgtacagagatatttatccaatgtgggctctagctgaatatcgaaggcgggttccattgccttccactgaagtttaatttagaatggtttgagcacgaaaaggcaaaggcattttcattaagattgaggcaaataagttgtgtgtaatcaagcttaatcaattttttcatattcctatgtttatttcctacatatattggtagaaaaattatttcaaaaaaaaaaaaaaaaaa

SEQ ID NO: 19 - DNA, 2766 bp, Glycine max
  misc_feature (1)..(2766): cDNA clone sah1c.pk002.n23 encoding an oxidosqualene cyclase
ttcatctcccacgcttcactttctccctccccctccctctccctctccctctccccaccccgagacctcaccctcccctccttctccctttcgccaccacaacgcccaacgtccacataagctagatgagatcaatctgaagcaaatggttataatttcaaaattttaagagtggaggacctgtgttgtgcacgttagagtgaatcgttcaagattaatccttaacaacctgaccaccaggaacaaccagctatcattttacattgaactagaaattcatttagaagatcaaagacaaaattttccgattaaaacgtacttaaattgaagaggggttgttggcattgtgcaccaaaaaggaaaaaaaatgtggaggttaaagatagcagatggagggaatgatccctatatatttagcacaaataattttgtggggaggcaaacatgggagtttgattctgaggcaggtaccgctgaggaacgagctcaaattgaagcagctcgtcaaaacttttatgaaaatcgcttcatggtcaaggcttgtggtgatcgactttggcggtttcagattttgagggaaaataatttcaaacaaacaataagtggcgtaaagatagaagatgatgagaaaattacatgcgagaaaattaggagcaccatgaagagggccactcattacctatcgtcactacagactagtgatggtcattggcctgctcatcttggaggttccctcttttttactccaccgttggtcatttgtttatatattacaggacatattgattctatattttcagaagagtatcgtaaagagattcttcgttacatatattaccaccagaacaaagatggaggttggggactacacatagaaggtcacagtatcatgttttgcactacactcaattatatatgcatgcgaattcttggagaaggacctaatggaggtcataacaatgcttgtgctaaagcaagaaagtggattcatgatcatggtggtgcaacacatataccttcatgggggaaattttggctttcggtacttggtatagttgattggtgtggaagcaacccaatgccgcctgaattttggatccttccttcttttctccctatgcatccgggtaaaatgtggtgttattgtcggttggtatacatgcccatgtcttatttgtatgggaagaaatttacgggtccaatcacaccgttagttgtaaatttgagagaagaactttttattcaaccttatgatgaaaatagttggaagaaagcacgtcataaatgtgcaaatgaagatctttactatccccatcattggatacaagatctattatgggatagtttgtatgtattcaccgagcctcttctaaattgttggcctttcaacaagttggttagagaaaaggcacttcaagtaacaatgaaacatattcattatgaagacgaaaatagtcggtatattgccatcgggtgtgtggaaaaggttctatgtatgcttgcttgttgggttgaagatccaaatggagatgctttcaagaagcatcttgcaaggatcccagattatttatgggtttctgaagatggaatgaccatgcagggtattggtactcaatcatgggatgttggtttcattgttcaagctttacttgctactaaccttatagatgattttggacctacaattgcaaaagctcacgatttcatcaagaaatctcaggtaagagaaaatccttcgggagattttaagagtatgtatcgtcacatttgtaaaggctcatggacccttgccgatagagatcatgcatggcaagtttctgataccactgcagaatgtttgaagtgttgtctacttttatcagtgctgccacaagatattgtgggagaaaaaatggaacttgaaaagttacatgattcaatcaatttgatactgtcacttcagagtaaaaatggaggtatgactgcgtgggagcccgcaggagcttataaatggttggaactactcaatcctacggaattttttgctgacatagtagttgagcacgaatatcttgaatgcactgcatcagcaattcaggttttagtgttgttcaaaaagctttaccctgagcatagaaaggaagagatagagaacttcattgctaaagcagtaacattcattgaagatacacaattagagaatggttcttggtatgggaattgggcagtttgtttcacttacagctcttggtttgcacttggaggtctagttgctgctggcaagacttacacaaattgtgttactattcgtaaagctgtgaaatttctactcaaaatacaaaataaggacggtgggtggggagagagttatctttcttgcccaaggaagatgtacgtacctcttgaaggaagtcgatcaaatgttgtacaaacatcatgggctctaatggctctaattcatgctgagcaggctgagagagatccaactccccttcatcatgcagcaaagttactcattaattctcagttagaagatggcgattggccccaacaagaaactcttggagtatacttgagaaattgcttggttcattactcattctatagaaatatttttccaatgtgggctttggctgaataccgcacaaatgttttattgccttcctttactatttaagttgaaaaattgtgagctcaaaaagataatgtcataccaataaaagtctagaaaaaaaaaagttggtaatgaagtttaataggcttattcataaaaaaaaaaaaaaaaaa

SEQ ID NO: 20 - DNA, 4690 bp, Artificial Sequence
  Recombinant DNA fragment 1025
  misc_feature (1190)..(1190): n is a, c, g, or t
cgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatgaaggtgttgaaagttctatttggctgctagcaaaggcttatgtagttgtgaatgactcgtgctatcatcaacttgtcagccattggttaaacactcatgcagttgttgagccattcatcatagcaacaaacaggcatctcagtgttgttcaccctatttataaactccttcaccctcactatcgtgacaccatgaacataaatggccttgctcggttatcactggtcaacgacggtggcgttatagaacaaacatttttgtggggaaggtattctgtggaaatgtctgctgtagtttacaaggattgggtttttacagatcaagcattgcctgctgatcttataaaaagaggaatggcaattgaggatccatcgtgccctcatggcattcgccttgtgatagaggactacccttatgctgttgatggacttgagatatgggatgctatcaagacatgggtccatgaatacgttttcttgtactacaaatcagatgacacacttagagaagatcctgaactccaagcctgctggaaagaactcgtagaggtgggtcatggagacaagaaaaatgagccatggtggcctaagatgcaaactcgtgaagagctagttgaagcttgcgctatcatcatatggactgcttcagcacttcatgcagctgttaattttggacagtatccctatggaggtttaatcttaaaccgtccaactcttagtaggcgattcatgcctgagaaaggttctgctgagtatgaggagctgaggaagaatccccagaaggcttacttgaagactattacaccaaagtttcagacccttattgacctttctgttatagaaatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggaattcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 21 - DNA, 2460 bp, Artificial Sequence
  gene expression-silencing cassette from pKS133
  misc_feature (1275)..(1275): n is a, c, g, or t
cgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaatgtgttattaacccttctcttcgaggatccaagcttgg

SEQ ID NO: 22 - DNA, 10 bp, Artificial Sequence
  sekf-annealing oligonucleotide linker
ggccgaattc

SEQ ID NO: 23 - DNA, 4351 bp, Artificial Sequence
  Recombinant DNA fragment 1028
  misc_feature (1190)..(1190): n is a, c, g, or t
cgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggaattcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 24 - DNA, 3964 bp, Artificial Sequence
  ALS selectable marker fragment
ggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 25 - protein, 656 aa, Glycine max
  MISC_FEATURE (1)..(656): soybean ALS resistant to sulfonylurea herbicides
MPHNTMAATASRTTRFSSSSSHPTFPKRITRSTLPLSHQTLTKPNHALKIKCSISKPPTAAPFTKEAPTTEPFVSRFASGEPRKGADILVEALERQGVTTVFAYPGGASMEIHQALTRSAAIRNVLPRHEQGGVFAAEGYARSSGLPGVCIATSGPGATNLVSGLADALMDSVPVVAITGQVARRMIGTDAFQETPIVEVSRSITKHNYLILDVDDIPRVVAEAFFVATSGRPGPVLIDIPKDVQQQLAVPNWDEPVNLPGYLARLPRPPAEAQLEHIVRLIMEAQKPVLYVGGGSLNSSAELRRFVELTGIPVASTLMGLGTFPIGDEYSLQMLGMHGTVYANYAVDNSDLLLAFGVRFDDRVTGKLEAFASRAKIVHIDIDSAEIGKNKQAHVSVCADLKLALKGINMILEEKGVEGKFDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQLEDRFYKSNRAHTYLGDPSSESEIFPNMLKFADACGIPAARVTKKEELRAAIQRMLDTPGPYLLDVIVPHQEHVLPMIPSNGSFKDVITEGDGRTRY

SEQ ID NO: 26 - protein, 4 aa, Artificial Sequence
  conserved “Subsequence B” from ALS
GQVP

SEQ ID NO: 27 - protein, 10 aa, Artificial Sequence
  conserved “Subsequence F” from ALS
  MISC_FEATURE (4)..(4): Xaa = Val or Met
GMVXQWEDRF

SEQ ID NO: 28 - protein, 5 aa, Artificial Sequence
  N-terminal amino acids added to ALS
MPHNT

SEQ ID NO: 29 - DNA, 8336 bp, Artificial Sequence
  Recombinant DNA fragment 1029
  misc_feature (1190)..(1190): n is a, c, g, or t
cgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggaattcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgaggggggtctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgacctgcaggcatgcaagcttcgctcgacgg

SEQ ID NO: 30 - DNA, 3072 bp, Artificial Sequence
  Recombinant DNA fragment KS136
  misc_feature (1191)..(1191): n is a, c, g, or t
cgcgccaagcttggatcctcgaagagaagggttaataacacacttttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgctgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaagcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 31 - DNA, 611 bp, Artificial Sequence
  FAD2 fragment in KS136
cggccgctgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaag

SEQ ID NO: 32 - DNA, 8948 bp, Artificial Sequence
  Recombinant DNA fragment PHP19853A
  misc_feature (5182)..(5182): n is a, c, g, or t
cgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcgcgcggccgcaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggtgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaagcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 33 - DNA, 2484 bp, Artificial Sequence
  LOX3, LOX2, FAD2 in recombinant DNA fragment PHP19853A
aattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggtgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaa

SEQ ID NO: 34 - DNA, 34 bp, Artificial Sequence
  Oligonucleotide primer TW108
cgatgcggccgcaattcctggagcattttatatc

SEQ ID NO: 35 - DNA, 40 bp, Artificial Sequence
  Oligonucleotide primer TW109
cactcgtgagcaatcactcacctctgaaagttaatccttc

SEQ ID NO: 36 - DNA, 40 bp, Artificial Sequence
  Oligonucleotide primer TW110
gaaggattaactttcagaggtgagtgattgctcacgagtg

SEQ ID NO: 37 - DNA, 33 bp, Artificial Sequence
  Oligonucleotide primer KS99
gaattcgcggccgcttaatctctgtccatagtt

SEQ ID NO: 38 - DNA, 8716 bp, Artificial Sequence
  Recombinant DNA fragment PHP19112A
  misc_feature (5182)..(5182): n is a, c, g, or t
cgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcgcgcggccgcaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatggccgcttagcgtagctgagatcaggcaggcacaaagggcagaaggcccagcaaccatccttgccattggaactgcaaacccaccaaaccgtgttgatcagagcacctatcctgattactacttcagaatcaccaacagtgaccacatgaccgagctcaaagagaaatttcagcgcatgtgtgacaagtctatgatcaagacgagatatatgtacctaaacgaagagatcttgaaagagaatccaaacatgtgtgcttacatggcaccttctttggatgctaggcaagacatggtggtggtagaggtaccaaagctagggaaagaggctgcagtaaaggccataaaggagtggggccagccaaagtcaaagattacccacttgatcttctgcaccactagcggtgtggacatgcctggtgctgattaccaactcaccaaacaattgggccttcgcccttatgtgaagaggtacatgatgtaccaacaaggttgctttgcaggtggcacggttcttcgtttggccaaggatttggctgagaacaacaagggtgcacgtgtgcttgttgtctgctctgagatcactgcagtcacgcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 39 - DNA, 2254 bp, Artificial Sequence
  LOX3, LOX2, CHS fragment in recombinant DNA fragment PHP19112A
aattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatggccgcttagcgtagctgagatcaggcaggcacaaagggcagaaggcccagcaaccatccttgccattggaactgcaaacccaccaaaccgtgttgatcagagcacctatcctgattactacttcagaatcaccaacagtgaccacatgaccgagctcaaagagaaatttcagcgcatgtgtgacaagtctatgatcaagacgagatatatgtacctaaacgaagagatcttgaaagagaatccaaacatgtgtgcttacatggcaccttctttggatgctaggcaagacatggtggtggtagaggtaccaaagctagggaaagaggctgcagtaaaggccataaaggagtggggccagccaaagtcaaagattacccacttgatcttctgcaccactagcggtgtggacatgcctggtgctgattaccaactcaccaaacaattgggccttcgcccttatgtgaagaggtacatgatgtaccaacaaggttgctttgcaggtggcacggttcttcgtttggccaaggatttggctgagaacaacaagggtgcacgtgtgcttgttgtctgctctgagatcactgcagtcacgc

SEQ ID NO: 40 - DNA, 30 bp, Artificial Sequence
  Oligonucleotide primer BM1
gcggccgcaattcctggagcattttatatc

SEQ ID NO: 41 - DNA, 35 bp, Artificial Sequence
  Oligonucleotide primer BM2
ctacgctaagcggccgcatgccttgacaagatctc

SEQ ID NO: 42 - DNA, 35 bp, Artificial Sequence
  Oligonucleotide primer BM3
cttgtcaaggcatgcggccgcttagcgtagctgag

SEQ ID NO: 43 - DNA, 30 bp, Artificial Sequence
  Oligonucleotide primer BM4
gcggccgcgtgactgcagtgatctcagagc

SEQ ID NO: 44 - DNA, 8906 bp, Artificial Sequence
  Recombinant DNA fragment PHP19113A
  misc_feature (5182)..(5182): n is a, c, g, or t
cgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcgcgcggccgcaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaagggagaagttgttgagggcgaggccagcggcgtcttcctcgacactttgcttgaattcgctgaggacgagaccatggagatcaaaattaccaaggagcaaatcaagggccttgttgtcgactttttctctgcagggacagattccacagcggtggcaacagagtgggcattggcagagctcatcaacaatcccagggtgttgcaaaaggctcgtgaggaggtctacagtgttgtgggcaaagatagactcgttgacgaagttgacactcaaaaccttccttacattagggccattgtgaaggagacattccgaatgcacccaccactcccagtggtcaaaagaaagtgcacagaagagtgtgagattaatgggtatgtgatcccagagggagcattggttcttttcaatgtttggcaagtaggaagggaccccaaatactgggacagaccatcagaattccgtcccgagaggttcttagaaactggtgctgaaggggaagcagggcctcttgatcttaggggccagcatttccaactcctcccatttgggtctgggaggagaatgtgccctggtgtcaatttggctacttcaggaatggcaacacttcttgcatctcttatccaatgctttgacctgcaagtgctgggccctcaaggacaaatattgaaaggtgatgatgccaaagttagcatggaagagagagctggcctcacagttccaagggcacatagtctcgtttgtgttccacttgcaaggatcggcgttgcatctaaactcctttcttaagcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 45 - DNA, 2442 bp, Artificial Sequence
  LOX2, LOX3, IFS in recombinant DNA fragment PHP19113A
aattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaagggagaagttgttgagggcgaggccagcggcgtcttcctcgacactttgcttgaattcgctgaggacgagaccatggagatcaaaattaccaaggagcaaatcaagggccttgttgtcgactttttctctgcagggacagattccacagcggtggcaacagagtgggcattggcagagctcatcaacaatcccagggtgttgcaaaaggctcgtgaggaggtctacagtgttgtgggcaaagatagactcgttgacgaagttgacactcaaaaccttccttacattagggccattgtgaaggagacattccgaatgcacccaccactcccagtggtcaaaagaaagtgcacagaagagtgtgagattaatgggtatgtgatcccagagggagcattggttcttttcaatgtttggcaagtaggaagggaccccaaatactgggacagaccatcagaattccgtcccgagaggttcttagaaactggtgctgaaggggaagcagggcctcttgatcttaggggccagcatttccaactcctcccatttgggtctgggaggagaatgtgccctggtgtcaatttggctacttcaggaatggcaacacttcttgcatctcttatccaatgctttgacctgcaagtgctgggccctcaaggacaaatattgaaaggtgatgatgccaaagttagcatggaagagagagctggcctcacagttccaagggcacatagtctcgtttgtgttccacttgcaaggatcggcgttgcatctaaactcctttcttaa

SEQ ID NO: 46 - DNA, 35 bp, Artificial Sequence
  Oligonucleotide primer BM8
ctcaacaacttctcccttgacaagatctctatcac

SEQ ID NO: 47 - DNA, 36 bp, Artificial Sequence
  Oligonucleotide primer BM9
agagatcttgtcaagggagaagttgttgagggcgag

SEQ ID NO: 48 - DNA, 32 bp, Artificial Sequence
  Oligonucleotide primer BM10
gcggccgcttaagaaaggagtttagatgcaac

SEQ ID NO: 49 - DNA, 8783 bp, Artificial Sequence
  Recombinant DNA fragment PHP19027A
  misc_feature (5182)..(5182): n is a, c, g, or t
cgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcgcgcggccgcaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaagatggcaccaacagccaagactctgacttacctggcccaggagaaaaccctagaatcgagcttcgttcgggacgaggaggagcgtcccaaggttgcctacaacgaattcagcgacgagatcccagtgatttctcttgccggaatcgacgaggtggatggacgcagaagagagatttgtgagaagatcgtggaggcttgcgagaattggggtatattccaggttgttgatcacggtgtggatcaacaactcgtggccgagatgacccgtctcgccaaagagttctttgctttgccaccggacgagaagcttcgttttgatatgtccggcgccaaaaagggtggattcattgtctccagccatctccaaggggaatcggtgcaggactggagagaaatagtgacatacttttcgtacccaaaaagagagagggactattcaaggtggccagacacgccagaagggtggagatcggtgactgaggaatacagcgacaaagtaatgggtctagcttgcaagctcatggaggtgttgtccgaagcaatggggttagagaaagagggtttaagcaaagcatgtgttgacatggaccagaaggtggtggttaattactaccccaaatgccctcaacctgacctcactcttggcctgaagcgccacacggatgcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 50 - DNA, 2319 bp, Artificial Sequence
  LOX2, LOX3, F3H in recombinant DNA fragment PHP19027A
aattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaagatggcaccaacagccaagactctgacttacctggcccaggagaaaaccctagaatcgagcttcgttcgggacgaggaggagcgtcccaaggttgcctacaacgaattcagcgacgagatcccagtgatttctcttgccggaatcgacgaggtggatggacgcagaagagagatttgtgagaagatcgtggaggcttgcgagaattggggtatattccaggttgttgatcacggtgtggatcaacaactcgtggccgagatgacccgtctcgccaaagagttctttgctttgccaccggacgagaagcttcgttttgatatgtccggcgccaaaaagggtggattcattgtctccagccatctccaaggggaatcggtgcaggactggagagaaatagtgacatacttttcgtacccaaaaagagagagggactattcaaggtggccagacacgccagaagggtggagatcggtgactgaggaatacagcgacaaagtaatgggtctagcttgcaagctcatggaggtgttgtccgaagcaatggggttagagaaagagggtttaagcaaagcatgtgttgacatggaccagaaggtggtggttaattactaccccaaatgccctcaacctgacctcactcttggcctgaagcgccacacggat

SEQ ID NO: 51 - DNA, 35 bp, Artificial Sequence
  Oligonucleotide primer BM11
ggctgttggtgccatcttgacaagatctctatcac

SEQ ID NO: 52 - DNA, 35 bp, Artificial Sequence
  Oligonucleotide primer BM12
agagatcttgtcaagatggcaccaacagccaagac

SEQ ID NO: 53 - DNA, 32 bp, Artificial Sequence
  Oligonucleotide primer BM13
gcggccgcatccgtgtggcgcttcaggccaag

SEQ ID NO: 54 - DNA, 9746 bp, Artificial Sequence
  Recombinant DNA fragment PHP19338A
  misc_feature (5182)..(5182): n is a, c, g, or t
cgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgcaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagagatcttgtcaaggcatgcaaccaccacaccacacagcacttcccttctcctcctccaccatgtcattgccaccgccgtcgctggtaacggcggcgactccgacggagcttccgatccggcagattccgggaagctacgggttcccgttgctgggccccatctcggatcggcttgactacttctggttccagaagccagagagcttcttcagaaagcgagtggagaaatacaagagcaccgtgttccgcaccaatgttcctccctccttccccttcttcgttaacgttaatcccaacgtaatcgcggttctcgacgtgaaatcattctcccacctcttcgacatggacctcgtcgacaagaaggatgttctcgtcggagactttgtccccagcgtcgccttcaccggaaacatgagagtcggcgtttaccaggacactaccgaacctcaacattccaaggtgaagaactacatcatggatattctgaaaagaagctcaggcatatgggtatcagaactagaatcaaacctagacacactgtcactcctcctcctccctctctcttccaaacacacatcctcaaccatggcagcaccatcttcagagacaaagtcaccatcatcctcgaacacgcagcttccgctgaaaccaatcccaggcagctacggaatgccgttttttggagcaataagcgacagacacaactacttctaccaccaaggacgcgacaagttcttcgcgacgaggattgaaaaacacaactccaccgtgatccgaaccaacatgcctccggggcccttcatctcctcggaccctcgtgtcgtcgcgttgttggacggtgcctccttcccgatcctcttcgacaacgacaaggtcgagaagctcaacgttctcgacggcaccttcatgccttccaccaagttcaccggcgggttccgcgtctgcgcctacctcgacaccaccgaacccaaccacgcactcatcaaacagttcttccttaacgtcctcgccaagcgaaaagactcttttgtccctctgttccgaaactgcctccaggagtcgttcgcggagattgaggaccagttgagtaaaaacaccaaggccagcttcaactcctccgtcggctccgccaccttcaacttcctcttccgtctcctctccgacaaagacccctccgaaaccataatcggctccgacggccccagcctggtccaaacctggctggcagctcagctggctcctctggccactctaggcttacccaggatcttcaactacgtggaggatttcttaatcaggtcaatcccctttccagcatggactgtcaaatccagctacaagaaactctacgaaggtctctcgacagcaggtactgcgattctggacgaagcggagcgcgtggggataaagagggacgaagcgtgccacaatcttgtgttcatgttatcgttcaacgcgcagggtgggttagtgaaccagtttccgattttgatcaagtggctaggactggctggggagggtttacacaagcagctcgcggaggagatcaggaccgttgttaaggacgaaggaggggtgagtctccgggcgttggatcagatgactttgaccaaatcagtggtgtatgaggtcctgaggatagcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 55 - DNA, 3286 bp, Artificial Sequence
  LOX3, LOX2, HPL1, HPL2, HPL3 fragments in recombinant DNA fragment PHP19338A
aattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagagatcttgtcaaggcatgcaaccaccacaccacacagcacttcccttctcctcctccaccatgtcattgccaccgccgtcgctggtaacggcggcgactccgacggagcttccgatccggcagattccgggaagctacgggttcccgttgctgggccccatctcggatcggcttgactacttctggttccagaagccagagagcttcttcagaaagcgagtggagaaatacaagagcaccgtgttccgcaccaatgttcctccctccttccccttcttcgttaacgttaatcccaacgtaatcgcggttctcgacgtgaaatcattctcccacctcttcgacatggacctcgtcgacaagaaggatgttctcgtcggagactttgtccccagcgtcgccttcaccggaaacatgagagtcggcgtttaccaggacactaccgaacctcaacattccaaggtgaagaactacatcatggatattctgaaaagaagctcaggcatatgggtatcagaactagaatcaaacctagacacactgtcactcctcctcctccctctctcttccaaacacacatcctcaaccatggcagcaccatcttcagagacaaagtcaccatcatcctcgaacacgcagcttccgctgaaaccaatcccaggcagctacggaatgccgttttttggagcaataagcgacagacacaactacttctaccaccaaggacgcgacaagttcttcgcgacgaggattgaaaaacacaactccaccgtgatccgaaccaacatgcctccggggcccttcatctcctcggaccctcgtgtcgtcgcgttgttggacggtgcctccttcccgatcctcttcgacaacgacaaggtcgagaagctcaacgttctcgacggcaccttcatgccttccaccaagttcaccggcgggttccgcgtctgcgcctacctcgacaccaccgaacccaaccacgcactcatcaaacagttcttccttaacgtcctcgccaagcgaaaagactcttttgtccctctgttccgaaactgcctccaggagtcgttcgcggagattgaggaccagttgagtaaaaacaccaaggccagcttcaactcctccgtcggctccgccaccttcaacttcctcttccgtctcctctccgacaaagacccctccgaaaccataatcggctccgacggccccagcctggtccaaacctggctggcagctcagctggctcctctggccactctaggcttacccaggatcttcaactacgtggaggatttcttaatcaggtcaatcccctttccagcatggactgtcaaatccagctacaagaaactctacgaaggtctctcgacagcaggtactgcgattctggacgaagcggagcgcgtggggataaagagggacgaagcgtgccacaatcttgtgttcatgttatcgttcaacgcgcagggtgggttagtgaaccagtttccgattttgatcaagtggctaggactggctggggagggtttacacaagcagctcgcggaggagatcaggaccgttgttaaggacgaaggaggggtgagtctccgggcgttggatcagatgactttgaccaaatcagtggtgtatgaggtcctgaggata

SEQ ID NO: 56 - DNA, 41 bp, Artificial Sequence
  Oligonucleotide primer BM14
gtgctgtgtggtgtggtggttgcatgccttgacaagatctc

SEQ ID NO: 57 - DNA, 41 bp, Artificial Sequence
  Oligonucleotide primer BM15
gagatcttgtcaaggcatgcaaccaccacaccacacagcac

SEQ ID NO: 58 - DNA, 38 bp, Artificial Sequence
  Oligonucleotide primer BM16
gaggagtgacagtgtgtctaggtttgattctagttctg

SEQ ID NO: 59 - DNA, 36 bp, Artificial Sequence
  Oligonucleotide primer BM17
gacacactgtcactcctcctcctccctctctcttcc

SEQ ID NO: 60 - DNA, 34 bp, Artificial Sequence
  Oligonucleotide primer BM18
gttgaagctggccttggtgtttttactcaactgg

SEQ ID NO: 61 - DNA, 40 bp, Artificial Sequence
  Oligonucleotide primer BM19
ttgagtaaaaacaccaaggccagcttcaactcctccgtcg

SEQ ID NO: 62 - DNA, 38 bp, Artificial Sequence
  Oligonucleotide primer BM20
gcggcctatcctcaggacctcatacaccactgatttgg

SEQ ID NO: 63 - DNA, 30 bp, Artificial Sequence; identical to SEQ ID NO: 40
  Oligonucleotide primer BM21

SEQ ID NO: 64 - DNA, 28 bp, Artificial Sequence
  Oligonucleotide primer BM22
gagggaggaggaggagtgacagtgtgtc

SEQ ID NO: 65 - DNA, 29 bp, Artificial Sequence
  Oligonucleotide primer BM23
atcaaacctagacacactgtcactcctcc

SEQ ID NO: 66 - DNA, 30 bp, Artificial Sequence
  Oligonucleotide primer BM24
gcggccgctatcctcaggacctcatacacc

SEQ ID NO: 67 - DNA, 9358 bp, Artificial Sequence
  Recombinant DNA fragment PHP19104A
  misc_feature (5182)..(5182): n is a, c, g, or t
cgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcgcgcggccgcaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggatgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcccttggttttttgtatgtatattacaggaaatcttgaatcagtatttccagaagaacatcgcaaagaaattcttcgttacacatattatcaccagaatgaagacggaggatggggactacacatagagggtcatagcactatgttttgtactgcactgaactatatatgcatgcgaatkcttggagaaggacctaatggaggtcatracaatgcttgtgctaaagcaagaaagtggattcatgatcatggtggtgcaacacatataccttcatgggggaaattttggctttcggtacttggtatagttgattggtgtggaagcaacccaatgccgcctgaattttggatccttccttcttttctccctatgcatccgggtaaaatgtggtgttattgtcggttggtatacatgcccatgtcttatttgtatgggaagaaatttacgggtccaatcacaccgttagttgtaaatttgagagaagaactttttattcaaccttatgatgaaaatagttggaagaaagcacgtcataaatgtgcaaatgaagatctttactatccccatcattggatacaagatctattatgggatagtttgtatgtattcaccgagcctcttctaaattgttggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 68 - DNA, 2894 bp, Artificial Sequence
  LOX3, LOX2, b-amyrin synthase, oxidosqualene cyclase in recombinant DNA fragment PHP19104A
aattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggatgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcccttggttttttgtatgtatattacaggaaatcttgaatcagtatttccagaagaacatcgcaaagaaattcttcgttacacatattatcaccagaatgaagacggaggatggggactacacatagagggtcatagcactatgttttgtactgcactgaactatatatgcatgcgaatkcttggagaaggacctaatggaggtcatracaatgcttgtgctaaagcaagaaagtggattcatgatcatggtggtgcaacacatataccttcatgggggaaattttggctttcggtacttggtatagttgattggtgtggaagcaacccaatgccgcctgaattttggatccttccttcttttctccctatgcatccgggtaaaatgtggtgttattgtcggttggtatacatgcccatgtcttatttgtatgggaagaaatttacgggtccaatcacaccgttagttgtaaatttgagagaagaactttttattcaaccttatgatgaaaatagttggaagaaagcacgtcataaatgtgcaaatgaagatctttactatccccatcattggatacaagatctattatgggatagtttgtatgtattcaccgagcctcttctaaattgttg

SEQ ID NO: 69 - DNA, 36 bp, Artificial Sequence
  Oligonucleotide primer BM5
cttcagcctccacatcctctgaaagttaatccttcc

SEQ ID NO: 70 - DNA, 30 bp, Artificial Sequence
  Oligonucleotide primer BM25
gcggccgccaacaatttagaagaggctcgg

SEQ ID NO: 71 - DNA, 30 bp, Artificial Sequence
  Oligonucleotide primer BM26
ttcttggagaaggacctaatggaggtcatg

SEQ ID NO: 72 - DNA, 30 bp, Artificial Sequence
  Oligonucleotide primer BM27
gcggccgcatgtggaggctgaagatagcag

SEQ ID NO: 73 - DNA, 30 bp, Artificial Sequence
  Oligonucleotide primer BM28
gtcatgacctccattaggtccttctccaag

SEQ ID NO: 74 - DNA, 30 bp, Artificial Sequence; identical to SEQ ID NO: 72
  Oligonucleotide primer BM29

SEQ ID NO: 75 - DNA, 30 bp, Artificial Sequence; identical to SEQ ID NO: 70
  Oligonucleotide primer BM30

SEQ ID NO: 76 - DNA, 35 bp, Artificial Sequence
  Oligonucleotide primer BM6
gattaactttcagaggatgtggaggctgaagatag

SEQ ID NO: 77 - DNA, 31 bp, Artificial Sequence
  Oligonucleotide primer BM7
gcggccgcaacaatttagaagaggctcggtg

SEQ ID NO: 78 - DNA, 9997 bp, Artificial Sequence
  Recombinant DNA fragment PHP19962A
  misc_feature (5184)..(5184): n is a, c, g, or t
ggcgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgctgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaaaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggatgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcccttggttttttgtatgtatattacaggaaatcttgaatcagtatttccagaagaacatcgcaaagaaattcttcgttacacatattatcaccagaatgaagacggaggatggggactacacatagagggtcatagcactatgttttgtactgcactgaactatatatgcatgcgaatkcttggagaaggacctaatggaggtcatracaatgcttgtgctaaagcaagaaagtggattcatgatcatggtggtgcaacacatataccttcatgggggaaattttggctttcggtacttggtatagttgattggtgtggaagcaacccaatgccgcctgaattttggatccttccttcttttctccctatgcatccgggtaaaatgtggtgttattgtcggttggtatacatgcccatgtcttatttgtatgggaagaaatttacgggtccaatcacaccgttagttgtaaatttgagagaagaactttttattcaaccttatgatgaaaatagttggaagaaagcacgtcataaatgtgcaaatgaagatctttactatccccatcattggatacaagatctattatgggatagtttgtatgtattcaccgagcctcttctaaattgttggcggccgcaagggcgaattctgcagatatccatcacactggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgac

SEQ ID NO: 79 - DNA, 3497 bp, Artificial Sequence
  FAD2, lox3, lox2, beta-amyrin synthase, oxidosqualene cyclase in recombinant DNA fragment PHP19962A
tgagtgattgctcacgagtgtggtcaccatgccttcagcaagtaccaatgggttgatgatgttgtgggtttgacccttcactcaacacttttagtcccttatttctcatggaaaataagccatcgccgccatcactccaacacaggttcccttgaccgtgatgaagtgtttgtcccaaaaccaaaatccaaagttgcatggttttccaagtacttaaacaaccctctaggaagggctgtttctcttctcgtcacactcacaatagggtggcctatgtatttagccttcaatgtctctggtagaccctatgatagttttgcaagccactaccacccttatgctcccatatattctaaccgtgagaggcttctgatctatgtctctgatgttgctttgttttctgtgacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattaaaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagaggatgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcccttggttttttgtatgtatattacaggaaatcttgaatcagtatttccagaagaacatcgcaaagaaattcttcgttacacatattatcaccagaatgaagacggaggatggggactacacatagagggtcatagcactatgttttgtactgcactgaactatatatgcatgcgaatkcttggagaaggacctaatggaggtcatracaatgcttgtgctaaagcaagaaagtggattcatgatcatggtggtgcaacacatataccttcatgggggaaattttggctttcggtacttggtatagttgattggtgtggaagcaacccaatgccgcctgaattttggatccttccttcttttctccctatgcatccgggtaaaatgtggtgttattgtcggttggtatacatgcccatgtcttatttgtatgggaagaaatttacgggtccaatcacaccgttagttgtaaatttgagagaagaactttttattcaaccttatgatgaaaatagttggaagaaagcacgtcataaatgtgcaaatgaagatctttactatccccatcattggatacaagatctattatgggatagtttgtatgtattcaccgagcctcttctaaattgttg

SEQ ID NO: 80 - DNA, 30 bp, Artificial Sequence
  Oligonucleotide primer BM31
gcggccgctgagtgattgctcacgagtgtg

SEQ ID NO: 81 - DNA, 40 bp, Artificial Sequence
  Oligonucleotide primer BM32
tataaaatgctccaggaattttaatctctgtccatagttg

SEQ ID NO: 82 - DNA, 42 bp, Artificial Sequence
  Oligonucleotide primer BM33
caactatggacagagattaaaattcctggagcattttatatc

SEQ ID NO: 83 - DNA, 30 bp, Artificial Sequence; identical to SEQ ID NO: 70
  Oligonucleotide primer BM34

SEQ ID NO: 84 - DNA, 27 bp, Artificial Sequence
  Oligonucleotide primer sense
atcttgtgttcatgttatcgttcaacg

SEQ ID NO: 85 - DNA, 24 bp, Artificial Sequence
  Oligonucleotide primer antisense
ggctcctccgtctggggtccgttg

SEQ ID NO: 86 - DNA, 4974 bp, Artificial Sequence
  Vector pKS133
  misc_feature (3951)..(3951): n is a, c, g, or t
ggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccgactcgacgatgagcgagatgaccagctcc

SEQ ID NO: 87 - DNA, 8966 bp, Artificial Sequence
  Vector pKS210
  misc_feature (1177)..(1177): n is a, c, g, or t
gatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcg

SEQ ID NO: 88 - DNA, 890 bp, Artificial Sequence
  Recombinant DNA fragment KSFAD2-hybrid
cggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaaggg

SEQ ID NO: 89 - DNA, 28 bp, Artificial Sequence
  Oligonucleotide primer KS1
gcggccgccggtcctctctctttccgtg

SEQ ID NO: 90 - DNA, 30 bp, Artificial Sequence
  Oligonucleotide primer KS2
tagagagagtaagtcctgcaagtactcctg

SEQ ID NO: 91 - DNA, 29 bp, Artificial Sequence
  Oligonucleotide primer KS3
caggagtacttgcaggacttactctctct

SEQ ID NO: 92 - DNA, 29 bp, Artificial Sequence
  Oligonucleotide primer KS4
gcggccggccccttctcggatgttccttc

SEQ ID NO: 93 - DNA, 9231 bp, Artificial Sequence
  Recombinant DNA fragment PHP21672A
  misc_feature (5182)..(5182): n is a, c, g, or t
cgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 94 - DNA, 2779 bp, Artificial Sequence
  2779 polynucleotide fragment comprising portions of FAD2-2, FAD2-1, LOX3, and LOX2
ggccgccggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggc

SEQ ID NO: 95 - DNA, 28 bp, Artificial Sequence
  Oligonucleotide primer BM35
gcggccgccggtcctctctctttccgtg

SEQ ID NO: 96 - DNA, 32 bp, Artificial Sequence
  Oligonucleotide primer BM36
aaatgctccaggaattcccttctcggatgttc

SEQ ID NO: 97 - DNA, 35 bp, Artificial Sequence
  Oligonucleotide primer BM37
catccgagaagggaattcctggagcattttatatc

SEQ ID NO: 98 - DNA, 29 bp, Artificial Sequence
  Oligonucleotide primer BM38
gcggccgccctctgaaagttaatccttcc

SEQ ID NO: 99 - DNA, 9874 bp, Artificial Sequence
  Recombinant DNA fragment PHP21676A
  misc_feature (5182)..(5182): n is a, c, g, or t
cgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcggatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacgg

SEQ ID NO: 100 - DNA, 3414 bp, Artificial Sequence
  3414 bp comprising portions of FAD2-2, FAD2-1, FAD3, LOX3, and LOX2
cggtcctctctctttccgtggcatggcaatctattgggctgtccagggttgcatccttactggtgtttgggtcattgcccatgagtgtggtcaccatgcattcagtgactaccagctgcttgatgatattgttggccttatcctccactccgctctcctagtcccgtacttttcatggaaatacagccatcgccgtcaccactccaacactggttctcttgagcgggatgaagtatttgtgccaaagcagaagtcctgtatcaagtggtactctaaataccttaacaatcctccaggcagagtcctcactcttgctgtcaccctcacacttggttggcccttgtacttggctttaaatgtttctggaaggccttatgatagatttgcttgccactatgacccatatggtcccatttactctgatcgtgaacgacttcaaatatatatatcagatgcaggagtacttgcaggacttactctctctaccgtgttgcaaccctgaaagggttggtttggctgctatgtgtttatggggtgcctttgctcattgtgaacggttttcttgtgactatcacatatttgcagcacacacactttgccttgcctcattacgattcatcagaatgggactggctgaagggagctttggcaactatggacagagattatgggattctgaacaaggtgtttcatcacataactgatactcatgtggctcaccatctcttctctacaatgccacattaccatgcaatggaggcaaccaatgcaatcaagccaatattgggtgagtactaccaatttgatgacacaccattttacaaggcactgtggagagaagcgagagagtgcctctatgtggagccagatgaaggaacatccgagaagggctcctccaccgtttaagattgcagaaatcagagcttcaataccaaaacattgctgggtcaagaatccatggagatccctcagttatgttctcagggatgtgcttgtaattgctgcattggtggctgcagcaattcacttcgacaactggcttctctggctaatctattgccccattcaaggcacaatgttctgggctctctttgttcttggacatgattgtggccatggaagcttttcagatagccctttgctgaatagcctggtgggacacatcttgcattcctcaattcttgtgccataccatggatggagaattagccacagaactcaccatcaaaaccatggacacattgagaaggatgagtcatgggttccattaacagagaagatttacaagaatctagacagcatgacaagactcattagattcactgtgccatttccattgtttgtgtatccaatttatttgttttcaagaagccccggaaaggaaggctctcacttcaatccctacagcaatctgttcccacccagtgagagaaaaggaatagcaatatcaacactgtgttgggctaccatgttttctctgcttatctatctctcattcataactagtccacttctagtgctcaagctctatggaattcctggagcattttatatcaagaattttatgcaaactgagtttttccttgtgagtttgactcttgaagacattccaaaccatggaagcatccactttgtttgcaattcgtggatttacaatgccaaactcttcaaaagtgaccgcattttctttgccaaccagacatatcttccaagtgagacaccagctccactagtcaaatatagagaagaagagttgcataatttaagaggagatggaactggagaacgcaaagagtgggaaaggatctatgattatgatgtctacaatgatttaggtgatccggataaaggtgaaaatcatgcccgtcctgttcttggaggaaatgacacctttccttatcctcgtagggggagaactggtagaaaaccaacaaggaaagatcctaatagtgagagtaggagcaatgatgtttatcttccaagagatgaggcttttggacacttgaagtcatctgactttcttacttatggactaaaatccgtatctcaaaatgttcttccattattgcaatctgcttttgatttgaatttcacaccccgtgagtttgatagctttgatgaagttcatggactctattcaggcggaattaagctgccaacagatataatcagcaagattagtccactacccgtgcttaaggaaatcttccgaactgatggtgaacaggcccttaagtttcctcctcctaaagtaattcaagtgagtaagtctgcatggatgactgatgaagaatttgcaagagaaatgcttgctggtgtaaatccaaacttgattcgttgtcttaaggatttccctccacgaagcaagctagatagccaagtctatggtgatcatactagtcaaataaccaaagaacacctagagcccaacttagaagggctcactgtagatgaggcaattcaaaacaagagattgttcctactagatcatcatgacccaatcatgccatatttgaggcgaataaatgcaacctccacaaaggcttatgctaccagaaccatccttttcctgaaaaatgacggaactttaaggccacttgccatagagttgagtttgccacatcctcagggagatcaatctggtgcttttagtcaagtttttctgcctgcagatcttatcaagagaggagtggcaattaaggatccatctgccccacatggacttcgacttctgatagaggactacccttatgctgttgatgggctagagatatgggctgcaattaagacatgggtccaagaatatgtgtccttgtactatgcaagagatgatgatgtcaaacctgattctgaactccaacagtggtggaaagaagctgtagagaaaggtcatggtgatctgaaagacaagccatggtggcctaagttgcaaacaattgaagagcttgttgaaatttgcaccattatcatatggactgcttcagccctccatgcagccgttaactttggtcaatatccatatggaggtttcattctgaatcgcccaacttcttctagaaggttgcttcctgagaaaggcaccccagaatatgaagaaatggtgaaaagtcatcaaaaggcttatttgagaactattacatcaaagtttcaaactctagttgacctttcagtgatagagatcttgtcaaggcatgcatctgatgaggtgtaccttggggagagggacaatccaaattggacatctgatacaagagcattagaggcttttaaaaggtttggaaataaactggcacaaattgagaataaactctcagagagaaacaacgatgagaaactgagaaaccgttgtggaccagttcaaatgccttatactctgcttttgccttctagtaaggaaggattaactttcagagg

SEQ ID NO: 101 - DNA, 31 bp, Artificial Sequence
  Oligonucleotide primer BM39
taaacggtggaggagcccttctcggatgttc

SEQ ID NO: 102 - DNA, 33 bp, Artificial Sequence
  Oligonucleotide primer BM40
gaacatccgagaagggctcctccaccgtttaag

SEQ ID NO: 103 - DNA, 28 bp, Artificial Sequence
  Oligonucleotide primer BM41
gcggccgcccatagagcttgagcactag

SEQ ID NO: 104 - DNA, 35 bp, Artificial Sequence
  Oligonucleotide primer BM42
taaaatgctccaggaattccatagagcttgagcac

SEQ ID NO: 105 - DNA, 37 bp, Artificial Sequence
  Oligonucleotide primer BM43
gctcaagctctatggaattcctggagcattttatatc

(19) United States
(12) Patent Application Publication
(10) Pub. No.: US 2006/0005276 A1
(43) Pub. Date: Jan. 5, 2006

(54) Transgenic soybean seeds having reduced activity of lipoxygenases

(76) Inventors: Saverio Carl Falco, Arden, DE (US); Brian McGonigle, Wilmington, DE (US); Carl A. Maxwell, Elkton, MD (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/076,733

(22) Filed: Mar. 9, 2005

Related U.S. Application Data
(60) Provisional application No. 60/552,502, filed on Mar. 12, 2004.
(60) Provisional application No. 60/556,248, filed on Mar. 25, 2004.

(51) Int. Cl. A01H 1/00; C12N 15/82; A01H 5/00

(52) U.S. Cl. 800/281; 800/312

(57) ABSTRACT

The present invention concerns a transgenic soybean plant producing seed having reduced activity of seed lipoxygenases, when compared to a soybean plant expressing wild type activity of native seed lipoxygenases, the transgenic soybean plant having a nucleic acid fragment from at least a portion of at least one soybean seed lipoxygenase gene, wherein the nucleic acid fragment is capable of suppressing expression of native seed lipoxygenases and has been introduced into the soybean plant by transformation. The present invention also concerns a transgenic soybean plant producing seed having reduced activity of seed lipoxygenases and a second native enzyme, when compared to a soybean plant expressing wild type activity of native seed lipoxygenases and the second native enzyme, the transgenic soybean plant having a first nucleic acid fragment from at least a portion of at least one soybean seed lipoxygenase gene, wherein the first nucleic acid fragment is capable of suppressing expression of said native seed lipoxygenases, and a second nucleic acid fragment from at least a portion of at least one second native enzyme gene, wherein the second nucleic acid fragment is capable of suppressing expression of the native second enzyme, wherein the first nucleic acid fragment and the second nucleic acid fragment have been introduced into the soybean plant by transformation, and wherein the second enzyme is selected from the group consisting of an enzyme of the lipid oxidation pathway, fatty acid desaturation pathway, phenylpropanoid pathway, triterpenoid pathway, and combinations thereof. Methods of suppressing wild type activity of native soybean seed lipoxygenases, alone or in combination with suppression of a second native enzyme are also embodied by the present invention.

This application claims priority benefit of U.S. Provisional Application No. 60/556,248, filed Mar. 25, 2004 and of U.S. Provisional Application No. 60/552,502, filed Mar. 12, 2004. The content of these Provisional Applications is hereby incorporated by reference in their entirety.

This invention is in the field of plant molecular biology. More specifically, this invention pertains to nucleic acid fragments useful in reducing the activity of seed lipoxygenases in transgenic soybeans. Included in the invention are transgenic soybean plants capable of producing seed having reduced activity of seed lipoxygenases and soybean plants capable of producing seed having reduced activity of lipoxygenases and reduced activity of a second enzyme of the lipid oxidation pathway, an enzyme of the fatty acid desaturation pathway, an enzyme of the phenylpropanoid pathway, an enzyme of the triterpenoid pathway, or combinations thereof.

### BACKGROUND OF THE INVENTION

Lipoxygenases are dioxygenases that catalyze, as a primary reaction, the hydroperoxidation, by molecular oxygen, of linoleic acid (18:2) and any other polyunsaturated lipids that contain a cis, cis-1,4-pentadiene moiety. Lipoxygenases (also referred to as LOX) are membrane-associated ubiquitous enzymes that catalyze the first step of a fatty acid metabolism pathway. Products of this pathway are found as signal molecules, involved in growth and development regulation, in senescence, and in response to pathogen invasion and wound stress (Rosahl (1996) Z. Naturforsch. (C) 51:123-138). Lipoxygenases with different specificities, subcellular location, and tissue-specific expression patterns have been identified in several plants including rice, barley, soybean, tomato, cucumber and potato.

Soybean seeds contain high levels of lipoxygenase. Three seed-expressed isozymes, designated lipoxygenases 1, 2 and 3 (also referred to as LOX1, LOX2, and LOX3), have been identified and well characterized enzymatically. The genes encoding the three soybean seed isozymes have been cloned and sequenced. However, no clear physiological role has yet been attributed to the soybean seed lipoxygenases (Siedow (1991) Annu. Rev. Plant Physiol. Plant Mol. Biol. 42:145-188).

Food products produced from soybeans have “beany” and “grassy” off-flavors that limit the potential for wider use of this economical and healthy source of protein. A great deal of research has been undertaken to understand the source of these off-flavors and considerable evidence has been accumulated which indicates that fatty acid breakdown products are a major source. It is believed that soybean seed lipoxygenases are major contributors to the generation of the off-flavors because soybeans contain high levels of polyunsaturated fatty acids and high levels of lipoxygenases. Lipoxygenases catalyze the first enzymatic step in the metabolic breakdown of the polyunsaturated fatty acids into off-flavor compounds such as C₆ aldehydes and alcohols. Soybeans lacking one or more of the seed lipoxygenase isozymes have been identified and shown to produce reduced amounts of fatty acid breakdown products (Hildebrand et al. (1981) J. Am. Oil Chem. Soc. 58:583-586; Pfeiffer et al. (1992) Crop Sci. 32:357-362). Soybeans lacking lipoxygenase isozymes 2 and 3 have been reported to have lower levels of off-flavor compounds and better taste (Kitamura et al. (1993) Trends Food Sci. Tech. 4:64-67). A soybean mutant lacking all three of the seed lipoxygenase isozymes has been obtained and shown to produce lower levels of many, not all, of the compounds associated with off-flavors (Kobayashi et al. (1995) J. Agric. Food Chem. 43:2449-2452). Soymilk made from soybeans lacking lipoxygenase isozymes 1, 2, and 3 was different in several flavor attributes from soymilk made from soybeans from normal lipoxygenase lines (Torres-Penaranda et al. (2001) J. Food Sci. 66:352-356).

While it has been possible to create a soybean line that lacks all three seed lipoxygenase isozymes (LOX1, LOX2, and LOX3), this line carries three recessive mutations, one in each of the three seed lipoxygenase genes, making breeding and commercial agricultural use of this line very difficult.

Polyunsaturated fatty acids are major precursors of the off-flavor compounds in soybean. The major polyunsaturated fatty acid in soybean, linoleic acid, is synthesized from the main product of the plastidial fatty acid biosynthesis, oleic acid, by the membrane bound FAD2. FAD2 is the microsomal oleoyl phosphatidylcholine desaturase (EC 1.3.1.35) that converts oleic acid to linoleic acid in a reaction that reduces molecular oxygen to water and requires the presence of NADH. U.S. Pat. No. 5,952,544 describes the isolation and use of a FAD2 gene from soybean to reduce the levels of polyunsaturated fatty acids in soybeans and Heppard et al. ((1996) Plant Physiol. 110:311-319) report the existence of two different fatty acid desaturases, designated FAD2-1 and FAD2-2.

The soybean FAD2-1 and FAD2-2 are delta-12 (Δ-12) desaturases that introduce a second double bond into oleic acid to form a linoleic acid, a polyunsaturated fatty acid. FAD2-1 is the major enzyme of this type in soybean seeds and reduction in the expression of FAD2-1 results in increased accumulation of oleic acid (18:1, or an 18 carbon fatty acid tail with a single double bond) and a corresponding decrease in polyunsaturated fatty acid content. Reduction of expression of FAD2-2 in combination with FAD2-1 leads to a greater accumulation of oleic acid and corresponding decrease in polyunsaturated fatty acid content.

FAD3 is a delta-15 (Δ-15) desaturase that introduces a third double bond into linoleic acid (18:2) to form linolenic acid (18:3) (Yadav et al. (1993) Plant Physiol. 103:467-476). Reduction of expression of FAD3 in combination with reduction of FAD2-1 and FAD2-2 leads to an even greater accumulation of oleic acid and corresponding decrease in polyunsaturated fatty acid content, especially linolenic acid.

In addition to compounds that are derived from fatty acid breakdown, soybeans are rich in a number of compounds derived from the phenylpropanoid pathway, most notably isoflavones. Isoflavones have been described as having bitter or astringent taste characteristics when consumed by humans. Huang et al. (1981) J. Food Sci. 47:19-23 and Okuba et al. (1992) Biosci. Biotech. Biochem. 56:99-103. Other phenylpropanoids, particularly flavanols and condensed tannins are also believed to impart taste characteristics on foods containing those compounds. The total isoflavone levels, as well as the distribution among different aglycones, is quite variable in soybean seeds and is affected by genetics and environmental conditions such as growing location and temperature during seed fill. Foods made from soybeans typically reflect the endogenous isoflavone composition, and as such genistein-derived isoflavone forms are the most abundant in most food products, while the daidzein-derived and the glycitein-derived forms are present in lower levels. PCT publication WO 00/44909 published on Aug. 03, 2000 describes the isolation and use of isoflavone synthase genes from soybean to alter the levels of isoflavones in soybeans.

Total saponin content varies somewhat by soybean cultivar, but is in the range of 0.25% of the seed dry weight. The physiological function of saponins in soybean seeds is not clear, but saponins and sapogenols purified from soybean seeds have been described as having bitter or astringent taste characteristics when consumed by humans. In an attempt to find the compound(s) possessing undesirable taste characteristics in dried pea, a natural products fractionation approach was taken leading to the purification of soyasponin I (a type of B group saponin) (Price, K. R. and Fenwick, G. R., J. Sci. Food Agric., 1984, 35, 887-892). However, the role that saponins play in the undesirable taste characteristics of soy food products is still under investigation.

In recent years, there has been interest in quinoa (Chenopodium quinoa) as an alternative food crop, in part because of its ability to grow in marginal conditions. Although widely used by the Incas, quinoa requires extensive post-harvest preparation in order to remove undesirable taste characteristics. Some of these characteristics have been removed by the development of sweet quinoa, which has significantly decreased levels of saponins and, thus, a decreased need for extensive post-harvest preparation. It seems likely that saponins will contribute to the undesirable taste characteristics of soyfood products, and reducing the saponin content of soybeans will result in better flavored food products derived from soybean. PCT publication WO 03/095615 published on Nov. 11, 2003 describes the isolation and use of oxidosqualene cyclase genes from soybean to alter the levels of saponins in soybeans.

### SUMMARY OF THE INVENTION

One embodiment of the invention comprises a transgenic soybean plant producing seed having reduced activity of seed lipoxygenases, when compared to a soybean plant expressing wild type activity of native seed lipoxygenases, the transgenic soybean plant having a nucleic acid fragment from at least a portion of at least one soybean seed lipoxygenase gene, wherein the nucleic acid fragment is capable of suppressing expression of the native seed lipoxygenases and has been introduced into the soybean plant by transformation.

Another embodiment of the invention comprises a transgenic soybean plant producing seed having reduced activity of seed lipoxygenases, when compared to a soybean plant expressing wild type activity of native seed lipoxygenases, the transgenic soybean plant having a first nucleic acid fragment from at least a portion of at least one soybean seed lipoxygenase gene, wherein the first nucleic acid fragment is capable of suppressing expression of the native seed lipoxygenases, and reduced activity of a second native enzyme selected from the group consisting of an enzyme of the lipid oxidation pathway, fatty acid desaturation pathway, phenylpropanoid pathway, triterpenoid pathway, and combinations thereof, when compared to a soybean plant expressing wild type activity of the second native enzyme, the transgenic soybean plant having a second nucleic acid fragment from at least a portion of at least one second native enzyme gene, wherein the second nucleic acid fragment is capable of suppressing expression of the second native enzyme, wherein the first nucleic acid fragment and the second nucleic acid fragment have been introduced into the soybean plant by transformation.

The present invention comprises soybean seed and plants wherein the second nucleic acid fragment corresponding to the second native enzyme is selected from the group consisting of fatty acid desaturase, beta-amyrin synthase, oxidosqualene cyclase, isoflavone synthase, chalcone synthase, flavanone 3-hydroxylase, hydroperoxide lyase, and combinations thereof. The second enzyme of the lipid oxidation pathway being suppressed may be hydroperoxide lyase. The enzyme of the fatty acid desaturation pathway may be selected from fatty acid desaturase 2 (either FAD2-1 or FAD2-2) and fatty acid desaturase 3 (FAD3). The enzyme of the phenylpropanoid pathway may be selected from isoflavone synthase, chalcone synthase, and flavanone 3-hydroxylase. The enzyme of the triterpenoid pathway may be selected from beta-amyrin synthase and oxidosqualene cyclase.

The present invention also includes a method of suppressing wild type activity of native soybean seed lipoxygenases comprising transforming plant tissue with a nucleic acid fragment from at least a portion of at least one soybean seed lipoxygenase gene, wherein the nucleic acid fragment is capable of suppressing expression of native soybean seed lipoxygenases, regenerating the plant tissue into a transgenic plant, growing the transgenic plant to produce transgenic seed, and evaluating said transgenic seed for suppression of soybean seed lipoxygenases when compared to seed having wild type activity of native soybean seed lipoxygenases.

Another embodiment of the invention comprises a method of suppressing wild type activity of native soybean seed lipoxygenases and a second native enzyme selected from the group consisting of an enzyme of the lipid oxidation pathway, the fatty acid desaturation pathway, the phenylpropanoid pathway, the triterpenoid pathway, and combinations thereof, comprising transforming plant tissue with a first nucleic acid fragment from at least a portion of at least one soybean seed lipoxygenase gene, wherein the nucleic acid fragment is capable of suppressing expression of native soybean seed lipoxygenases, and a second nucleic acid fragment from at least a portion of at least one second enzyme gene, wherein the second nucleic acid fragment is capable of suppressing expression of the second native enzyme, regenerating the plant tissue into a transgenic plant, growing the transgenic plant to produce transgenic seed, measuring activity of lipoxygenases in the transgenic seed, and evaluating said transgenic seed for suppression of soybean seed lipoxygenases and suppression of said second native enzyme when compared to seed having wild type activity of soybean seed lipoxygenases and said second native enzyme.

Also included in the invention are the grains from the transgenic plants of the invention. Soybean protein product prepared from grain is also an embodiment of the invention. Oil, feed, food, and industrial products are also contemplated by the present invention.

### BRIEF DESCRIPTION OF THE FIGURES AND SEQUENCE LISTINGS

The invention can be more fully understood from the following detailed description and the accompanying Figures and Sequence Listing that form part of this application.

FIG. 1 shows a phylogenetic tree of soybean lipoxygenases. The tree was created using amino acid sequences for soybean lipoxygenase 1 (NCBI GI NO: 18675), soybean lipoxygenase 2 (NCBI GI NO: 170014), soybean lipoxygenase 3 (NCBI GI NO: 1794172), soybean lipoxygenase 4 (NCBI GI NO: 585418), soybean lipoxygenase 5 (NCBI GI NO: 7433153), soybean lipoxygenase vlxC (NCBI GI NO: 7433154), and soybean lipoxygenase 7 (NCBI GI NO: 7433156). This phylogenetic tree shows that soybean seed lipoxygenases 1 and 2 are closely related, while soybean seed lipoxygenase 3 is a more distant relative.

FIG. 2 shows a depiction of plasmid pKS133.

FIG. 3 shows a depiction of plasmid pKS210.

The sequence descriptions and Sequence Listing attached hereto comply with the rules governing nucleotide and/or amino acid sequence disclosures in patent applications as set forth in 37 C.F.R. §1.821-1.825.

SEQ ID NO:1 is the nucleotide sequence of the cDNA insert in clone sde4c.pk0003.c8 encoding an entire soybean lipoxygenase 1 (LOX1).

SEQ ID NO:2 is the nucleotide sequence of the cDNA insert in clone se4.pk0007.e7 encoding an entire soybean lipoxygenase 2 (LOX2).

SEQ ID NO:3 is the nucleotide sequence of the cDNA insert in clone sgs1c.pk002.g4 encoding an entire soybean lipoxygenase 3 (LOX3).

SEQ ID NO:4 is the nucleotide sequence having NCBI General Identifier No.18674 and encoding an entire soybean LOX1.

SEQ ID NO:5 is the nucleotide sequence having NCBI General Identifier No.170013 and encoding an entire soybean LOX2.

SEQ ID NO:6 is the nucleotide sequence having NCBI General Identifier No.1794171 and encoding an entire soybean LOX3.

SEQ ID NO:7 is the nucleotide sequence of the longest stretch of continuous identical nucleotides shared by three currently known soybean seed lipoxygenases (LOX1, LOX2, and LOX3).

SEQ ID NO:8 is the nucleotide sequence of the longest stretch of continuous identical nucleotides shared by LOX1 and LOX2.

SEQ ID NO:9 is the nucleotide sequence of the cDNA insert in clone sr1.pk0097.b11 encoding an entire soybean chalcone synthase (CHS).

SEQ ID NO:10 is the nucleotide sequence of the cDNA insert in clone sdp3c.pk017.j17 encoding an entire hydroperoxide lyase (HPL) used to prepare plasmid HPL3.

SEQ ID NO:11 is the amino acid sequence corresponding to the translation of nucleotides 49 through 1470 of SEQ ID NO:10.

SEQ ID NO:12 is the nucleotide sequence of the cDNA insert in clone sdp4c.pk015.e22 encoding an entire HPL used to prepare plasmid HPL2.

SEQ ID NO:13 is the amino acid sequence corresponding to the translation of nucleotides 44 through 1477 of SEQ ID NO:12.

SEQ ID NO:14 is the nucleotide sequence of the cDNA insert in clone sgs4c.pk002.f8 encoding an entire HPL used to prepare plasmid HPL1.

SEQ ID NO:15 is the amino acid sequence corresponding to the translation of nucleotides 52 through 1512 of SEQ ID NO:14.

SEQ ID NO:16 is the nucleotide sequence of the cDNA insert in clone sgs1c.pk006.o20 encoding an entire soybean isoflavone synthase (IFS).

SEQ ID NO:17 is the nucleotide sequence of the cDNA insert in clone sfl1.pk0040.g11 encoding an entire flavanone 3-hydroxylase (F3H).

SEQ ID NO:18 is the nucleotide sequence of the cDNA insert in clone src3c.pk024.m11 encoding an entire β-amyrin synthase (BAM).

SEQ ID NO:19 is the nucleotide sequence of the cDNA insert in clone sah1c.pk002.n23 encoding an entire oxidosqualene cyclase (OSC).

SEQ ID NO:20 is the nucleotide sequence of recombinant DNA fragment 1025 which comprises a portion of the soybean LOX3 gene.

SEQ ID NO:21 is the nucleotide sequence of the seed-specific gene expression-silencing cassette from pKS133 which comprises nucleotides for a Kti3 promoter and terminator bordering a string of nucleotides that promote formation of a stem structure which are surrounding a unique Not I restriction endonuclease site.

SEQ ID NO:22 is the nucleotide sequence of the self-annealing oligonucleotide linker used to generate the unique Eco RI at the Not I site of pKS133.

SEQ ID NO:23 is the nucleotide sequence of recombinant DNA fragment 1028 which comprises a portion of the soybean LOX3 gene and a portion of the soybean LOX2 gene.

SEQ ID NO:24 is the nucleotide sequence of the ALS selectable marker recombinant DNA fragment. This recombinant DNA fragment comprises a promoter operably linked to a nucleotide fragment encoding a soybean acetolactate synthase to which mutations have been introduced to make it resistant to treatment with sulfonylurea herbicides.

SEQ ID NO:25 is the amino acid sequence of the soybean herbicide-resistant ALS including mutations in subsequences B and F.

SEQ ID NO:26 is the wild type amino acid sequence of conserved ALS “subsequence B” disclosed in U.S. Pat. No. 5,013,659.

SEQ ID NO:27 is the wild type amino acid sequence of conserved ALS “subsequence F” disclosed in U.S. Pat. No. 5,013,659.

SEQ ID NO:28 is the amino acid sequence of the additional five amino acids introduced during cloning at the amino-terminus of the soybean ALS.

SEQ ID NO:29 is the nucleotide sequence of recombinant DNA fragment 1029 which comprises a seed LOX expression silencing cassette and a selectable marker gene.

SEQ ID NO:30 is the nucleotide sequence of recombinant DNA fragment KS136 which comprises a FAD2-1 seed-specific gene expression silencing cassette.

SEQ ID NO:31 is the nucleotide sequence of the approximately 600 nucleotide fragment obtained by digesting the FAD2-1 gene with Nco I and used to prepare KS136.

SEQ ID NO:32 is the nucleotide sequence of recombinant DNA fragment PHP19853A which includes a gene expression-silencing cassette designed to silence seed LOX and FAD2-1 linked to the ALS selectable marker recombinant DNA fragment.

SEQ ID NO:33 is the nucleotide sequence of the 2480 polynucleotide fragment comprising about 1880 nucleotides from recombinant DNA fragment 1028 which includes about 1360 nucleotides from the soybean LOX3 gene and 520 nucleotides from the soybean LOX2 gene, and 600 nucleotides from the FAD2-1 gene and used to prepare recombinant DNA fragment PHP19853A.

SEQ ID NO:34 is the nucleotide sequence of oligonucleotide primer TW108 used to amplify a 1.9 kb DNA fragment using recombinant DNA fragment 1028 as template.

SEQ ID NO:35 is the nucleotide sequence of oligonucleotide primer TW109 used to amplify a 1.9 kb DNA fragment using recombinant DNA fragment 1028 as template.

SEQ ID NO:36 is the nucleotide sequence of oligonucleotide primer TW 10 used to amplify a 0.6 kb DNA fragment using recombinant DNA fragment KS136 as template.

SEQ ID NO:37 is the nucleotide sequence of oligonucleotide primer KS99 used to amplify a 0.6 kb DNA fragment using recombinant DNA fragment KS136 as template.

SEQ ID NO:38 is the nucleotide sequence of recombinant DNA fragment PHP19112A which contains a gene expression silencing cassette designed to silence expression of seed LOX and CHS linked to the ALS selectable marker recombinant DNA fragment.

SEQ ID NO:39 is the nucleotide sequence of an approximately 2250 nucleotide fragment comprising about 1140 nucleotides from the soybean LOX3 gene, 520 nucleotides from the soybean LOX2 gene, and 586 nucleotides from a soybean CHS gene used to prepare recombinant DNA fragment PHP19112A.

SEQ ID NO:40 is the nucleotide sequence of oligonucleotide primer BM1 used to amplify a portion of recombinant DNA fragment 1028.

SEQ ID NO:41 is the nucleotide sequence of oligonucleotide primer BM2 used to amplify a portion of recombinant DNA fragment 1028.

SEQ ID NO:42 is the nucleotide sequence of oligonucleotide primer BM3 used to amplify a portion of the cDNA insert in clone sr1.pk0097.b11.

SEQ ID NO:43 is the nucleotide sequence of oligonucleotide primer BM4 used to amplify a portion of the cDNA insert in clone sr1.pk0097.b11.

SEQ ID NO:44 is the nucleotide sequence of recombinant DNA fragment PHP19113A which comprises a gene expression silencing cassette designed to silence soybean seed LOX and IFS linked to the ALS selectable marker gene.

SEQ ID NO:45 is the nucleotide sequence of the 2440 nucleotide fragment comprising about 1140 nucleotides from the soybean LOX3 gene and 520 nucleotides from the soybean LOX2 gene, and 786 nucleotides from a soybean IFS gene present in recombinant DNA fragment PHP19113A.

SEQ ID NO:46 is the nucleotide sequence of the oligonucleotide primer BM8 used to amplify a portion of recombinant DNA fragment 1028.

SEQ ID NO:47 is the nucleotide sequence of the oligonucleotide primer BM9 used to amplify a portion of clone sgs1c.pk006.o20.

SEQ ID NO:48 is the nucleotide sequence of the oligonucleotide primer BM10 used to amplify a portion of clone sgs1c.pk006.o20.

SEQ ID NO:49 is the nucleotide sequence of recombinant DNA fragment PHP19027A which comprises a LOX-F3H gene expression silencing cassette linked to the ALS selectable marker gene.

SEQ ID NO:50 is the nucleotide sequence of the approximately 2320 nucleotide fragment comprising about 1140 nucleotides from the soybean LOX3 gene, 520 nucleotides from the soybean LOX2 gene, and 663 nucleotides from soybean clone sfl1.pk0040.g11.

SEQ ID NO:51 is the nucleotide sequence of oligonucleotide primer BM11 used to amplify a portion of recombinant DNA fragment 1028.

SEQ ID NO:52 is the nucleotide sequence of oligonucleotide primer BM12 used to amplify a portion of clone sfl1.pk0040.g11.

SEQ ID NO:53 is the nucleotide sequence of oligonucleotide primer BM13 used to amplify a portion of clone sfl1.pk0040.g11.

SEQ ID NO:54 is the nucleotide sequence of recombinant DNA fragment PHP19338A which comprises a LOX-HPL gene expression silencing cassette linked to the ALS selectable marker gene.

SEQ ID NO:55 is the nucleotide sequence of an approximately 3290 nucleotide fragment comprising about 1140 nucleotides from the soybean LOX3 gene, 520 nucleotides from the soybean LOX2 gene, and approximately 1626 nucleotides from the soybean HPL genes.

SEQ ID NO:56 is the nucleotide sequence of oligonucleotide primer BM14 used to amplify a portion of recombinant DNA 1028.

SEQ ID NO:57 is the nucleotide sequence of oligonucleotide primer BM15 used to amplify a portion of the cDNA insert in clone sdp3c.pk017.j17.

SEQ ID NO:58 is the nucleotide sequence of oligonucleotide primer BM16 used to amplify a portion of the cDNA insert in clone sdp3c.pk07.j17.

SEQ ID NO:59 is the nucleotide sequence of oligonucleotide primer BM17 used to amplify a portion of the cDNA insert in clone sgs4c.pk002.f8.

SEQ ID NO:60 is the nucleotide sequence of oligonucleotide primer BM18 used to amplify a portion of the cDNA insert in clone sgs4c.pk002.f8.

SEQ ID NO:61 is the nucleotide sequence of oligonucleotide primer BM19 used to amplify a portion of the cDNA insert in clone sdp4c.pk0015.e22.

SEQ ID NO:62 is the nucleotide sequence of oligonucleotide primer BM20 used to amplify a portion of the cDNA insert in clone sdp4c.pk0015.e22.

SEQ ID NO:63 is the nucleotide sequence of oligonucleotide primer BM21 used to amplify a portion of clone pAB.

SEQ ID NO:64 is the nucleotide sequence of oligonucleotide primer BM22 used to amplify a portion of clone pAB.

SEQ ID NO:65 is the nucleotide sequence of oligonucleotide primer BM23 used to amplify a portion of clone pCD.

SEQ ID NO:66 is the nucleotide sequence of oligonucleotide primer BM24 used to amplify a portion of clone pCD.

SEQ ID NO:67 is the nucleotide sequence of recombinant DNA fragment PHP19104A which comprises a LOX-β-amyrin synthase gene expression silencing cassette linked to the ALS selectable marker gene.

SEQ ID NO:68 is the nucleotide sequence of an approximately 2900 nucleotide fragment comprising about 1880 nucleotides from recombinant DNA fragment 1028 that includes fragments of the soybean LOX3 and LOX2 genes, followed by about 570 nucleotides from the cDNA insert in clone src3c.pk024.m11 and about 450 nucleotides from the cDNA insert in clone sah1c.pk002.n23.

SEQ ID NO:69 is the nucleotide sequence of oligonucleotide primer BM5 used to amplify a portion of recombinant DNA fragment 1028.

SEQ ID NO:70 is the nucleotide sequence of oligonucleotide primer BM25 used to amplify a portion of the cDNA insert in clone sah1c.pk002.n23.

SEQ ID NO:71 is the nucleotide sequence of oligonucleotide primer BM26 used to amplify a portion of the cDNA insert in clone sah1c.pk002.n23.

SEQ ID NO:72 is the nucleotide sequence of oligonucleotide primer BM27 used to amplify a portion of the cDNA insert in clone src3c.pk0024.m11.

SEQ ID NO:73 is the nucleotide sequence of oligonucleotide primer BM28 used to amplify a portion of the cDNA insert in clone src3c.pk0024.ml 1.

SEQ ID NO:74 is the nucleotide sequence of oligonucleotide primer BM29 used in amplifying fragment AC18.

SEQ ID NO:75 is the nucleotide sequence of oligonucleotide primer BM30 used in amplifying fragment AC18.

SEQ ID NO:76 is the nucleotide sequence of oligonucleotide primer BM6 used to amplify AC18.

SEQ ID NO:77 is the nucleotide sequence of oligonucleotide primer BM7 used to amplify AC18.

SEQ ID NO:78 is the nucleotide sequence of recombinant DNA fragment PHP19962A which comprises a LOX, β-amyrin synthase, oxidosqualene cyclase, and FAD2-1 gene expression silencing cassette linked to the ALS selectable marker gene.

SEQ ID NO:79 is the 3500 nucleotide fragment comprising about 610 nucleotides from the soybean FAD2-1 gene, about 1880 nucleotides from recombinant DNA fragment 1028 that includes fragments of the soybean LOX3 and LOX2 genes, followed by about 570 nucleotides from the cDNA insert in clone src3c.pk024.ml 1 and about 450 nucleotides from the cDNA insert in clone sah1c.pk002.n23.

SEQ ID NO:80 is the nucleotide sequence of oligonucleotide primer BM31 used to amplify a portion of recombinant DNA fragment KS136.

SEQ ID NO:81 is the nucleotide sequence of oligonucleotide primer BM32 used to amplify a portion of recombinant DNA fragment KS136.

SEQ ID NO:82 is the nucleotide sequence of oligonucleotide primer BM33 used to amplify a portion of recombinant DNA fragment PHP19112A.

SEQ ID NO:83 is the nucleotide sequence of oligonucleotide primer BM34 used to amplify a portion of recombinant DNA fragment PHP19112A.

SEQ ID NO:84 is the nucleotide sequence of primer Sense used to amplify HPL mRNA.

SEQ ID NO:85 is the nucleotide sequence of primer Antisense used to amplify HPL mRNA.

SEQ ID NO:86 is the nucleotide sequence of plasmid pKS133.

SEQ ID NO:87 is the nucleotide sequence of plasmid pKS210.

SEQ ID NO:88 is the nucleotide sequence of recombinant DNA fragment KSFAD2-hybrid which contains about 470 nucleotides from the soybean FAD2-2 gene and 420 nucleotides from the soybean FAD2-1 gene.

SEQ ID NO:89 is the nucleotide sequence of oligonucleotide primer KS1 used to amplify about 470 nucleotides from the soybean FAD2-2 gene.

SEQ ID NO:90 is the nucleotide sequence of oligonucleotide primer KS2 used to amplify about 470 nucleotides of the soybean FAD2-2 gene.

SEQ ID NO:91 is the nucleotide sequence of oligonucleotide primer KS3 used to amplify about 420 nucleotides of the soybean FAD2-1 gene.

SEQ ID NO:92 is the nucleotide sequence of oligonucleotide primer KS4 used to amplify about 420 nucleotides of the soybean FAD2-1 gene.

SEQ ID NO:93 is the nucleotide sequence of recombinant DNA fragment PHP21672A which contains a gene expression silencing cassette designed to silence expression of seed lipoxygenases (LOX) and both the FAD2-1 and FAD2-2 genes linked to the ALS selectable marker gene.

SEQ ID NO:94 is the nucleotide sequence of the approximately 2779 polynucleotide fragment comprising about 470 nucleotides from the soybean FAD2-2 gene, 420 nucleotides from the soybean FAD2-1 gene, and about 1880 nucleotides from the soybean LOX3 and LOX2 genes.

SEQ ID NO:95 is the nucleotide sequence of oligonucleotide primer BM35 used to amplify an approximately 0.9 Kb fragment from recombinant DNA fragment KSFAD2-hybrid.

SEQ ID NO:96 is the nucleotide sequence of oligonucleotide primer BM36 used to amplify an approximately 0.9 Kb fragment from recombinant DNA fragment KSFAD2-hybrid.

SEQ ID NO:97 is the nucleotide sequence of oligonucleotide primer BM37 used to amplify an approximately 1.9 kb DNA fragment from recombinant DNA fragment 1028.

SEQ ID NO:98 is the nucleotide sequence of oligonucleotide primer BM38 used to amplify an approximately 1.9 kb DNA fragment from recombinant DNA fragment 1028.

SEQ ID NO:99 is the nucleotide sequence of recombinant DNA fragment PHP21676A which comprises a gene expression silencing cassette designed to silence expression of seed lipoxygenases (LOX), the FAD2-1 and FAD2-2 genes, and the FAD3 gene, linked to the ALS selectable marker recombinant DNA fragment.

SEQ ID NO:100 is the nucleotide sequence of the approximately 3414 polynucleotide fragment comprising about 470 nucleotides from the soybean FAD2-2 gene, 420 nucleotides from the soybean FAD2-1 gene, 643 nucleotides from the soybean FAD3 gene, and about 1880 nucleotides from the soybean LOX3 and LOX2 genes.

SEQ ID NO:101 is the nucleotide sequence of oligonucleotide primer BM39 used to amplify an approximately 0.9 kb fragment from recombinant DNA fragment KSFAD2-hybrid.

SEQ ID NO:102 is the nucleotide sequence of oligonucleotide primer BM40 used to amplify an approximately 0.65 kb DNA fragment from plasmid XF1.

SEQ ID NO:103 is the nucleotide sequence of oligonucleotide plasmid BM41 used to amplify an approximately 0.65 kb DNA fragment from plasmid pXF1.

SEQ ID NO:104 is the nucleotide sequence of primer BM42 used to amplify an approximately 1.5 kb DNA fragment comprising a portion of the soybean FAD2-2 gene, a portion of the soybean FAD2-1 gene, and a portion of the soybean FAD3 gene.

SEQ ID NO:105 is the nucleotide sequence of primer BM43 used to amplify an approximately 1.9 kb DNA fragment comprising portions of the LOX2 and LOX3 genes from recombinant DNA fragment 1028.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

Definitions

In the context of this disclosure, a number of terms shall be utilized.

The terms “lipoxygenase” and “LOX” are used interchangeably herein. These terms refer to any member of a group of enzymes that catalyze the hydroperoxidation of polyunsaturated fatty acids in the first step of fatty acid metabolite synthesis. In the higher plant lipoxygenase pathway, linoleic acid and linolenic acid are oxygenated by the action of lipoxygenase (LOX) to produce hydroperoxide fatty acids. These hydroperoxide fatty acids undergo other transformations among which are the conversion to methyl esters by the action of allene oxide synthase (AOS) and cleavage between the hydroperoxide carbon and the neighboring double bond by the action of hydroperoxide lyases (HPLs).

The terms “hydroperoxide lyase” and “HPL” are used interchangeably herein. Hydroperoxide lyases process 9-hydroperoxides into C9 aldehydes and C9 aldoacids and 13-hydroperoxides into C6 aldehydes and C12 aldoacids. The aldehydes formed in these reactions can be further modified by alcohol dehydrogenases (ADH) to produce alcohols (Grechkin (1998) Prog. Lipid Res. 37:317-352). Both, 13-hydroperoxide lyase (13-HPL) and 9-hydroperoxide lyase (9-HPL) activities have been detected in soybean, pea, cucumber, and alfalfa seedlings, soybean and pea seeds, and cucumber fruits suggesting that different enzymes are specific for different functions. HPLs with high specificity for 13-hydroperoxides have been isolated, among others, from alfalfa (Noordermeer et al. (2000) Eur. J. Biochem 267:2473-2482), Arabidopsis leaves (Bate et al. (1998) Plant Phys. 117:1393-1400), cucumber hypocotyls (Matsui et al. (2000) FEBS Left. 481:183-188), bell pepper fruits (Matsui et al. (1996) FEBS Left. 394:21-24), and tomato fruits (Howe et al. (2000) Plant Phys. 123:711-724). Up to date, HPLs that act on 9- and 13-hydroperoxides with a preference for 9-hydroperoxides have been isolated from cucumber hypocotyls (Matsui et al. (2000) FEBS Left. 481:183-188) and from melon fruit (Tijet et. al. (2001) Arch. Biochem. Biophys. 386:281-289). These HPLs shows high amino acid similarity to AOSs although they do not show any detectable AOS activity.

The terms “fatty acid desaturase” and “FAD” are used interchangeably herein and refer to membrane bound microsomal oleoyl- and linoleoyl-phosphatidylcholine desaturases that convert oleic acid to linoleic acid and linoleic acid to linolenic acid, respectively, in reactions that reduce molecular oxygen to water and require the presence of NADH. Two soybean fatty acid desaturases, designated FAD2-1 and FAD2-2, are Δ-12 desaturases that introduce a second double bond into oleic acid to form a linoleic acid, a polyunsaturated fatty acid. FAD2-1 is the major enzyme of this type in soybean seeds and reduction in the expression of FAD2-1 results in increased accumulation of oleic acid (18:1, or an 18 carbon fatty acid tail with a single double bond) and a corresponding decrease in polyunsaturated fatty acid content. Reduction of expression of FAD2-2 in combination with FAD2-1 leads to a greater accumulation of oleic acid and corresponding decrease in polyunsaturated fatty acid content. FAD3 is a Δ-15 desaturase that introduces a third double bond into linoleic acid (18:2) to form linolenic acid (18:3). Reduction of expression of FAD3 in combination with reduction of FAD2-1 and FAD2-2 leads to an even greater accumulation of oleic acid and corresponding decrease in polyunsaturated fatty acid content, especially linolenic acid.

Isoflavonoids represent a class of secondary metabolites produced in legumes by a branch of the phenylpropanoid pathway and include such compounds as isoflavones, isoflavanones, rotenoids, pterocarpans, isoflavans, quinone derivatives, 3-aryl-4-hydroxy-coumarins, 3-arylcoumarins, isoflav-3-enes, coumestans, alpha-methyldeoxybenzoins, 2-arylbenzofurans, isoflavanol, coumaronochromone and the like. Free isoflavones rarely accumulate to high levels in soybeans. Instead they are usually conjugated to carbohydrates or organic acids. Soybean seeds contain three types of isoflavones aglycones, glucosides, and malonylglucosides. Each isoflavone type is found in three different forms: daidzein, genistein, and glycitein form the aglycones; daidzin, genistin, and glycitin form the glucosides; and 6″-O-malonyidaidzin, 6″-O-malonylgenistin and 6″-O-malonylglycitin form the malonylglucosides. During processing, acetylglucoside forms are produced: 6″-O-acetyldaidzin, 6″-O-acetyl genistin, and 6″-O-acetyl glycitin. The content of isoflavonoids in soybean seeds is quite variable and is affected by both genetics and environmental conditions such as growing location and temperature during seed fill (Tsukamoto, C., et al. (1995) J. Agric. Food Chem. 43:1184-1192; Wang, H. and Murphy, P. A. (1994) J. Agric. Food Chem. 42:1674-1677). The genistein-derived isoflavone forms make up the most abundant group in soybean seeds and most food products, while the daidzein and the glycitein forms are present in lower levels (Murphy, P. A. (1999) J. Agric. Food Chem. 47:2697-2704).

The terms “chalcone synthase” and “CHS” are used interchangeably herein. Chalcone synthase (CHS) is a member of a plant-specific polyketide synthase family that, in the phenylpropanoid pathway, catalyzes multiple rounds of condensation with 4-coumaryl CoA to produce chalcone (reviewed by Jez, J. M. et al.(2000) Biochemistry 39:890-902).

The terms “isoflavone synthase” and “IFS” are used interchangeably herein. Isoflavone synthase (IFS) catalyzes the first step in the phenylpropanoid branch that commits metabolic intermediates to the synthesis of isoflavonoids. In this central reaction, 2S-flavanone is converted into an isoflavonoid such as genistein and daidzein. The reaction involves a P450 monoxygenase-mediated conversion of the 2S-flavanone to a 2-hydroxyisoflavanone, followed by conversion to the isoflavonoid. This last step is possibly mediated by a soluble dehydratase (Kochs, G. and Grisenbach, H. (1985) Eur. J. Biochem. 155:311-318). However, the 2-hydroxyisoflavanone intermediate was described as unstable and could convert directly to genistein.

The terms “flavanone 3-hydroxylase” and “F3H” are used interchangeably herein. The enzyme flavanone 3-hydroxylase (F3H; EC 1.14.11.9) catalyzes the conversion of flavanones to dihydroflavonols, which are intermediates in the biosynthesis of flavonols, anthocyanidins, catechins and proanthocyanidins. This enzyme is also referred to as naringenin 3-dioxygenase, and naringenin, 2-oxoglutarate 3-dioxygenase, among others. In soybean, both F3H and IFS compete for naringenin as a substrate and it is not clear how this competition is regulated. Though the branch initiated by isoflavone synthase that leads to synthesis of isoflavonoids is mainly limited to the legumes, the remainder of the phenylpropanoid pathway occurs in other plant species.

The terpenoids, which are composed of the five-carbon isoprenoids, constitute the largest family of natural products with over 22,000 individual compounds of this class having been described. The terpenoids (hemiterpenes, monoterpenes, sesquiterpenes, diterpenes, triterpenes, tetraterpenes, polyterpenes, and the like) play diverse functional roles in plants as hormones, photosynthetic pigments, electron carriers, mediators of polysaccharide assembly, and structural components of membranes. Plant terpenoids are found in resins, latex, waxes, and oils.

Two molecules of farnesyl pyrophosphate are joined head-to-head to form squalene, a triterpene, in the first dedicated step towards sterol biosynthesis. Squalene is then converted to 2,3-oxidosqualene which, in photosynthetic organisms, may be converted to the 30 carbon, 4-ring structure, cycloartenol or to the 5-ring structure, β-amyrin. Cycloartenol is formed by the enzyme cycloartenol synthase (EC 5.4.99.8), also called 2,3-epoxysqualene-cycloartenol cyclase. The basic nucleus of cycloartenol can be further modified by reactions such as desaturation or demethylation to form the common sterol backbones such as stigmasterol and sitosterol, which can be modified further.

Oxidosqualene cyclases (OCS) catalyze the cyclization of 2,3-oxidosqualene to form various polycyclic skeletons including one or more of lanosterol, lupeol, cycloartenol, isomultiflorenol, β-amyrin, and α-amyrin. The non-cycloartenol producing oxidosqualene cyclase activities are different, although evolutionarily related, to cycloartenol synthases (Kushiro, T., et al. (1998) Eur. J. Biochem. 256:238-244). β-amyrin synthase (BAM) catalyzes the cyclization of 2,3-oxidosqualene to β-amyrin and is therefore an example of an oxidosqualene cyclase. The basic β-amyrin ring structure may be modified in much the same manner as is the cycloartenol structure to give classes of sapogenins, also known as sapogenols. Saponins are glycosylated sapogenins and may play a defense role against pathogens in plant tissues.

The term enzyme “activity” refers to the ability of an enzyme to convert a substrate to a product. For example, lipoxygenases convert a fatty acid to hydroperoxide fatty acids.

The terms “nucleic acid fragment,” “polynucleotide,” and “isolated nucleic acid fragment” are used interchangeably herein. These terms encompass nucleotide fragments and the like. A nucleic acid fragment may be a polymer of RNA or DNA that is single- or double-stranded, that optionally contains synthetic, non-natural or altered nucleotide bases. A nucleic acid fragment in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “homology,” “homologous,” “substantially similar,” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of nucleic acid fragments such as deletion or insertion of one or more nucleotides that do not substantially alter the functional properties of the resulting nucleic acid fragment relative to the fragment of interest. It is therefore understood, as those skilled in the art will appreciate, that the nucleic acid fragments mentioned herein encompass more than the specific exemplary sequences.

“Gene” refers to a nucleic acid fragment that expresses a specific protein. A gene encompasses regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, and arranged in a manner different than that found in nature. A “foreign” gene refers to a gene not normally found in the host organism, which is introduced into the host organism by gene transfer. Foreign genes can comprise native genes inserted into a non-native organism, or chimeric genes. A “transgene” is a gene that has been introduced into the genome by a transformation procedure. An “allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

“Coding sequence” refers to a DNA fragment that codes for a polypeptide having a specific amino acid sequence. “Regulatory sequences” refer to nucleotides located upstream (5′ non-coding sequences), within, or downstream (3′ non-coding sequences) of a coding sequence, and which influence the transcription, RNA processing, stability, or translation of the associated coding sequence. Regulatory sequences may include, and are not limited to, promoters, translation leader sequences, introns, and polyadenylation recognition sequences. “Promoter” refers to a region of DNA capable of controlling the expression of a coding sequence or functional RNA. The promoter sequence consists of proximal and more distal upstream elements. These upstream elements are often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters.” New promoters of various types useful in plant cells are constantly being discovered; numerous examples may be found in the compilation by Okamuro, J. K., and Goldberg, R. B. (1989) Biochemistry of Plants 15:1-82.

Any seed-specific promoter can be used in accordance with the method of the invention. Thus, the origin of the promoter chosen to drive expression of the recombinant DNA fragment is not critical as long as it is capable of accomplishing the invention by transcribing enough RNA from the desired nucleic acid fragment(s) in the seed.

A plethora of promoters is described in WO 00/18963, published on Apr. 6, 2000, the disclosure of which is hereby incorporated by reference. Examples of seed-specific promoters include, and are not limited to, the promoter for soybean Kunitz trysin inhibitor (Kti3, Jofuku and Goldberg (1989) Plant Cell 1:1079-1093) β-conglycinin (Chen et al. (1989) Dev. Genet. 10: 112-122), the napin promoter, and the phaseolin promoter.

The term “operably linked” refers to the association of nucleic acid fragments on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The “translation leader sequence” refers to a polynucleotide fragment located between the promoter of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Mol. Biotechnol. 3:225-236).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript. “Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro.

“Antisense RNA” refers to an RNA transcript that is complementary to all or part of a target primary transcript or mRNA, and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated, yet has an effect on cellular processes. The terms “complement” and “reverse complement” are used interchangeably herein with respect to mRNA transcripts, and are meant to define the antisense RNA of the message.

The term “recombinant” refers to an artificial combination of two otherwise separated segments of sequence, e.g., by chemical synthesis or by the manipulation of isolated segments of nucleic acid fragments by genetic engineering techniques.

The terms “recombinant construct,” “expression construct,” “chimeric construct,” “construct,” “recombinant DNA construct,” and recombinant DNA fragment are used interchangeably herein and are nucleic acid fragments. A recombinant construct comprises an artificial combination of nucleic acid fragments, including and not limited to regulatory and coding sequences that are not found together in nature. For example, a chimeric construct may comprise regulatory sequences and coding sequences that are derived from different sources, or regulatory sequences and coding sequences derived from the same source, and arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

The term “recombinant DNA construct” refers to a DNA construct assembled from nucleic acid fragments obtained from different sources. The types and origins of the nucleic acid fragments may be very diverse. A “recombinant DNA construct” includes and is not limited to the following combinations: a) nucleic acid fragment corresponding to a promoter operably linked to at least one nucleic acid fragment encoding a selectable marker, followed by a nucleic acid fragment corresponding to a terminator, b) a nucleic acid fragment corresponding to a promoter operably linked to a nucleic acid fragment capable of producing a stem-loop structure, and followed by a nucleic acid fragment corresponding to a terminator, and c) any combination of a) and b) above. In the stem-loop structure at least one nucleic acid fragment that is capable of suppressing expression of a native gene comprises the “loop” and is surrounded by nucleic acid fragments capable of producing a stem.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989. Transformation methods are well known to those skilled in the art and are described below.

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

The term “expression,” as used herein, refers to the production of a functional end-product e.g., a mRNA or a protein (precursor or mature).

“Mature” protein refers to a post-translationally processed polypeptide; i.e., one from which any pre- or pro-peptides present in the primary translation product have been removed. “Precursor” protein refers to the primary product of translation of mRNA; i.e., with pre- and pro-peptides still present. Pre- and pro-peptides may be and are not limited to intracellular localization signals.

“Stable transformation” refers to the transfer of a nucleic acid fragment into a genome of a host organism, including nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms.

As stated herein, “suppression” refers to the reduction of the level of enzyme activity detectable in a transgenic plant when compared to the level of enzyme activity detectable in a plant with the native enzyme. The level of enzyme activity in a plant with the native enzyme is referred to herein as “wild type” activity. The term “suppression” includes lower, reduce, decline, decrease, inhibit, eliminate and prevent. This reduction may be due to the decrease in translation of the native mRNA into an active enzyme. It may also be due to the transcription of the native DNA into decreased amounts of mRNA and/or to rapid degradation of the native mRNA. The term “native enzyme” refers to an enzyme that is produced naturally in the desired cell.

Suppression of enzymes in plants may be accomplished by any one of many methods known in the art which include the following. “Cosuppression” refers to the production of sense RNA transcripts capable of suppressing the expression of identical or substantially similar native genes (U.S. Pat. No. 5,231,020). Co-suppression constructs in plants have been previously designed by focusing on overexpression of a nucleic acid sequence having homology to a native mRNA, in the sense orientation, which results in the reduction of all RNA having homology to the overexpressed sequence (see Vaucheret et al. (1998) Plant J. 16:651-659; and Gura (2000) Nature 404:804-808). “Antisense inhibition” refers to the production of antisense RNA transcripts capable of suppressing the expression of the target protein. Plant viral sequences may be used to direct the suppression of proximal mRNA encoding sequences (PCT Publication WO 98/36083 published on Aug. 20, 1998). “Hairpin” structures that incorporate all, or part, of an mRNA encoding sequence in a complementary orientation resulting in a potential “stem-loop” structure for the expressed RNA have been described (PCT Publication WO 99/53050 published on Oct. 21, 1999). In this case the stem is formed by polynucleotides corresponding to the gene of interest inserted in either sense or anti-sense orientation with respect to the promoter and the loop is formed by some polynucleotides of the gene of interest, which do not have a complement in the construct. This increases the frequency of cosuppression or silencing in the recovered transgenic plants. For review of hairpin suppression see Wesley, S. V. et al. (2003) Methods in Molecular Biology, Plant Functional Genomics: Methods and Protocols 236:273-286. A construct where the stem is formed by at least 30 nucleotides from a gene to be suppressed and the loop is formed by a random nucleotide sequence has also effectively been used for suppression (WO 99/61632 published on Dec. 2, 1999). The use of poly-T and poly-A sequences to generate the stem in the stem-loop structure has also been described (WO 02/00894 published Jan. 3, 2002). Yet another variation includes using synthetic repeats to promote formation of a stem in the stem-loop structure. Transgenic organisms prepared with such recombinant DNA fragment show reduced levels of the protein encoded by the polynucleotide from which the nucleotide fragment forming the loop is derived as described in PCT Publication WO 02/00904, published Jan. 3, 2002. The use of constructs that result in dsRNA has also been described. In these constructs convergent promoters direct transcription of gene-specific sense and antisense RNAs inducing gene suppression (see for example Shi, H. et al. (2000) RNA 6:1069-1076; Bastin, P. et al. (2000) J. Cell Sci. 113:3321-3328; Giordano, E. et al. (2002) Genetics 160:637-648; LaCount, D. J. and Donelson, J. E. U.S. patent application No. 20020182223, published Dec. 5, 2002;Tran, N. et al. (2003) BMC Biotechnol. 3:21; and Applicant's U.S. Provisional Application No. 60/578,404, filed Jun. 9, 2004).

Other methods for suppressing an enzyme include, but are not limited to, use of polynucleotides that may form a catalytic RNA or may have ribozyme activity (U.S. Pat. No. 4,987,071 issued Jan. 22, 1991), and micro RNA (also called miRNA) interference (Javier et al. (2003) Nature 425:257-263).

The sequences of the polynucleotide fragments used for suppression do not have to be 100% identical to the sequences of the polynucleotide fragment found in the gene to be suppressed. For example, suppression of all the subunits of the soybean seed storage protein β-conglycinin has been accomplished using a polynucleotide derived from a portion of the gene encoding the a subunit (U.S. Pat. No. 6,362,399). β-conglycinin is a heterogeneous glycoprotein composed of varying combinations of three highly negatively charged subunits identified as α, α′ and β. The polynucleotide sequences encoding the α and α′ subunits are about 85% identical to each other while the polynucleotide sequences encoding the β subunit are about 75 to about 80% identical to the α and α′ subunits, respectively. Thus, polynucleotides that are at least about 75% identical to a region of the polynucleotide that is target for suppression have been shown to be effective in suppressing the desired target. The polynucleotide may be at least about 80% identical, at least about 90% identical, at least about 95% identical, or about 100% identical to the desired target sequence.

A “portion capable of suppressing expression” of a native gene refers to a portion or subfragment of an isolated nucleic acid fragment in which the ability to alter gene expression or produce a certain phenotype is retained whether or not the fragment or subfragment may be translated into an active enzyme. For example, the fragment or subfragment may be used in the design of chimeric genes or recombinant DNA fragments to produce the desired phenotype in a transformed plant. Chimeric genes may be designed for use in suppression by linking a nucleic acid fragment or subfragment thereof, whether or not it is translated into an active enzyme, in the sense or antisense orientation relative to a plant promoter sequence. Recombinant DNA fragments may be designed to comprise nucleic acid fragments capable of promoting formation of a stem-loop structure. In a stem-loop structure either the loop or the stem comprises a portion of the gene to be suppressed. The nucleic acid fragment should have a stretch of at least about 20 contiguous nucleotides that are identical to the gene to be suppressed. The stretch of contiguous nucleotides may be any number, from at least about 20, or about 32, to the size of the entire gene to be suppressed.

Methods for transforming dicots and obtaining transgenic plants have been published, among others, for cotton (U.S. Pat. No. 5,004,863, U.S. Pat. No. 5,159,135); soybean (U.S. Pat. No. 5,569,834, U.S. Pat. No. 5,416,011); Brassica (U.S. Pat. No. 5,463,174); peanut (Cheng et al. (1996) Plant Cell Rep. 15:653-657, McKently et al. (1995) Plant Cell Rep. 14:699-703); papaya (Ling, K. et al. (1991) Bio/technology 9:752-758); and pea (Grant et al. (1995) Plant Cell Rep. 15:254-258). For a review of other commonly used methods of plant transformation see Newell, C. A. (2000) Mol. Biotechnol. 16:53-65. One of these methods of transformation uses Agrobacterium rhizogenes (Tepfler, M. and Casse-Delbart, F. (1987) Microbiol. Sci. 4:24-28). Transformation of soybeans using direct delivery of DNA has been published using PEG fusion (PCT publication WO 92/17598), electroporation (Chowrira, G. M. et al. (1995) Mol. Biotechnol. 3:17-23; Christou, P. et al. (1987) Proc. Natl. Acad. Sci. U.S.A. 84:3962-3966), microinjection, or particle bombardment (McCabe, D. E. et. al. (1988) BiolTechnology 6:923; Christou et al. (1988) Plant Physiol. 87:671-674).

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In.: Methods for Plant Molecular Biology, (Eds.), Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. The regenerated plants may be self-pollinated. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide(s) is cultivated using methods well known to one skilled in the art.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant DNA fragments and recombinant expression constructs and the screening and isolating of clones, (see for example, Sambrook et al. (1989) Molecular Cloning: A Laboratory Manual, Cold Spring Harbor Press; Maliga et al. (1995) Methods in Plant Molecular Biology, Cold Spring Harbor Press; Birren et al. (1998) Genome Analysis: Detecting Genes, 1, Cold Spring Harbor, N.Y.; Birren et al. (1998) Genome Analysis: Analyzing DNA, 2, Cold Spring Harbor, N.Y.; Plant Molecular Biology: A Laboratory Manual, eds. Clark, Springer, N.Y. (1997)).

The terms “soy” and “soybean” are used interchangeably herein. Within the scope of the invention are soybean plants (Glycine soja or Glycine max), seeds, and plant parts obtained from such transformed plants. Also within the scope of the invention are soybean products derived from the transformed plants such as grain, protein products, oils, and products including such soybean products like feed and foodstuffs. Plant parts include differentiated and undifferentiated tissues, including and not limited to, roots, stems, shoots, leaves, pollen, seeds, tumor tissue, and various forms of cells and cultures such as single cells, protoplasts, embryos, and callus tissue. The plant tissue may be in plant or in organ, tissue or cell culture.

Included within the scope of this invention are soybean products that include protein isolates, protein concentrates, food products, feed products, etc. Methods for obtaining such products are well known to those skilled in the art. For example soybean protein products can be obtained in a variety of ways. Conditions typically used to prepare soy protein isolates have been described by (Cho, et al, (1981) U.S. Pat. No. 4,278,597; Goodnight, et al. (1978) U.S. Pat. No. 4,072,670). Soy protein concentrates are produced by three basic processes: acid leaching (at about pH 4.5), extraction with alcohol (about 55-80%), and denaturing the protein with moist heat prior to extraction with water. Conditions typically used to prepare soy protein concentrates have been described by Pass ((1975) U.S. Pat. No. 3,897,574) and Campbell et al. ((1985) in New Protein Foods, ed. by Altschul and Wilcke, Academic Press, Vol. 5, Chapter 10, Seed Storage Proteins, pp 302-338).

“Soybean-containing products” can be defined as those items produced of seeds from a suitable plant which are used in feeds, foods and/or beverages. For example, “soy protein products” can include, and are not limited to, those items listed in Table 1.

TABLE 1
Soy Protein Products Derived from Soybean Seeds^{a}

Whole Soybean Products
Roasted Soybeans
Baked Soybeans
Soy Sprouts
Soy Milk
Specialty Soy Foods/Ingredients
Soy Milk
Tofu
Tempeh
Miso
Soy Sauce
Hydrolyzed Vegetable Protein
Whipping Protein
Processed Soy Protein Products
Full Fat and Defatted Flours
Soy Grits
Soy Hypocotyls
Soybean Meal
Soy Milk
Soy Protein Isolates
Soy Protein Concentrates
Textured Soy Proteins
Textured Flours and Concentrates
Textured Concentrates
Textured Isolates
^{a}See Soy Protein Products: Characteristics, Nutritional Aspects and Utilization (1987). Soy Protein Council.

“Processing” refers to any physical and chemical methods used to obtain the products listed in Table 1 and includes, and is not limited to, heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992). Products referred to as being “high protein” or “low protein” are those as described by these Standard Specifications. “NSI” refers to the Nitrogen Solubility Index as defined by the American Oil Chemists' Society Method Ac4 41. “KOH Nitrogen Solubility” is an indicator of soybean meal quality and refers to the amount of nitrogen soluble in 0.036 M KOH under the conditions as described by Araba and Dale ((1990) Poult. Sci. 69:76-83). “White” flakes refer to flaked, dehulled cotyledons that have been defatted and treated with controlled moist heat to have an NSI of about 85 to 90. This term can also refer to a flour with a similar NSI that has been ground to pass through a No. 100 U.S. Standard Screen size. “Cooked” refers to a soy protein product, typically a flour, with an NSI of about 20 to 60. “Toasted” refers to a soy protein product, typically a flour, with an NSI below 20. “Grits” refer to defatted, dehulled cotyledons having a U.S. Standard screen size of between No. 10 and 80. “Soy Protein Concentrates” refer to those products produced from dehulled, defatted soybeans by three basic processes: acid leaching (at about pH 4.5), extraction with alcohol (about 55-80%), and denaturing the protein with moist heat prior to extraction with water. Conditions typically used to prepare soy protein concentrates have been described by Pass ((1975) U.S. Pat. No. 3,897,574; Campbell et al., (1985) in New Protein Foods, ed. by Altschul and Wilcke, Academic Press, Vol. 5, Chapter 10, Seed Storage Proteins, pp 302-338). “Extrusion” refers to processes whereby material (grits, flour or concentrate) is passed through a jacketed auger using high pressures and temperatures as a means of altering the texture of the material. “Texturing” and “structuring” refer to extrusion processes used to modify the physical characteristics of the material. The characteristics of these processes, including thermoplastic extrusion, have been described previously (Atkinson (1970) U.S. Pat. No. 3,488,770, Horan (1985) In New Protein Foods, ed. by Altschul and Wilcke, Academic Press, Vol. 1A, Chapter 8, pp 367-414). Moreover, conditions used during extrusion processing of complex foodstuff mixtures that include soy protein products have been described previously (Rokey (1983) Feed Manufacturing Technology III, 222-237; McCulloch, U.S. Pat. No. 4,454,804).

Also, within the scope of this invention are food, food supplements, food bars, and beverages that have incorporated therein a soybean-derived product of the invention. The beverage can be in a liquid or in a dry powdered form.

The foods to which the soybean-derived product of the invention can be incorporated/added include almost all foods/beverages. For example, there can be mentioned meats such as ground meats, emulsified meats, marinated meats, and meats injected with a soybean-derived product of the invention. Included may be beverages such as nutritional beverages, sports beverages, protein-fortified beverages, juices, milk, milk alternatives, and weight loss beverages. Mentioned may also be cheeses such as hard and soft cheeses, cream cheese, and cottage cheese. Included may also be frozen desserts such as ice cream, ice milk, low fat frozen desserts, and non-dairy frozen desserts. Finally, yogurts, soups, puddings, bakery products, salad dressings, spreads, and dips (such as mayonnaise and chip dips) may be included. The soybean-derived product can be added in an amount selected to deliver a desired dose to the consumer of the food and/or beverage.

In still another aspect this invention concerns a method of producing an soybean-derived product which comprises: (a) cracking the seeds obtained from transformed plants of the invention to remove the meats from the hulls; and (b) flaking the meats obtained in step (a) to obtain the desired flake thickness.

Yet another aspect of the present invention is directed to a method of suppressing wild type activity of native soybean seed lipoxygenases comprising transforming plant tissue with a nucleic acid fragment from at least a portion of at least one soybean seed lipoxygenase gene, wherein the nucleic acid fragment is capable of suppressing expression of native soybean seed lipoxygenases, regenerating the plant tissue into a transgenic plant, growing the transgenic plant to produce transgenic seed, and evaluating said transgenic seed for suppression of soybean seed lipoxygenases when compared to seed having wild type activity of native soybean seed lipoxygenases.

The method of suppressing expression of native soybean seed lipoxygenases and a second native enzyme selected from the group consisting of an enzyme of the lipid oxidation pathway, the fatty acid desaturation pathway, the phenylpropanoid pathway, the triterpenoid pathway, and combinations thereof is another embodiment of the present invention. The method comprises transforming plant tissue with a first nucleic acid fragment from at least a portion of at least one soybean seed lipoxygenase gene, wherein the nucleic acid fragment is capable of suppressing expression of native soybean seed lipoxygenases, and a second nucleic acid fragment from at least a portion of at least one second enzyme gene, wherein the second nucleic acid fragment is capable of suppressing expression of the second native enzyme, regenerating said plant tissue into a transgenic plant, growing the transgenic plant to produce transgenic seed, and evaluating said transgenic seed for suppression of soybean seed lipoxygenases and suppression of said second native enzyme when compared to seed having wild type activity of soybean seed lipoxygenases and said second native enzyme.

In order to carry out the present invention, cDNAs encoding enzymes involved in the lipid oxidation pathway, the fatty acid desaturation pathway, the phenylpropanoid pathway, and the triterpenoid pathway are identified. A portion or portions of each cDNA may then be used to prepare recombinant DNA constructs designed to suppress each enzyme of interest. The recombinant DNA constructs are introduced into somatic soybean embryos and transgenic soybean plants are regenerated. Various methods of transforming cells are known in the art and include Agrobacterium rhizogenes, direct delivery of DNA using PEG fusion, electroporation, microinjection (Rakoczy-Trojanowska, M. (2002) Cell Mol Biol Lett. 7:849-858) or particle gun bombardment, plant virus-mediated transformation (see, U.S. Pat. No. 6,369,296 and U.S. Pat. No. 6,635,805), and liposome-mediated transformation (Rakoczy-Trojanowska, id.).

Only transformed cells are typically capable of surviving a period on selection media. Assays employed to determine the levels of LOX1 activity in soybean embryos, seed chips, or bulk seed include methods known to skilled artisans and include and are not limited to assays developed for somatic embryo extracts, seed chip extracts, and bulk seed extracts. Such assays include methods such as spectrophotometric assays, SDS-polyacrylamide gel electrophoresis, and immunological assays, e.g. “western” blot or ELISA.

Transgenic soybean plants resulting from a transformation with a recombinant DNA are assayed to reveal plants with no detectable LOX1 activity. The recombinant DNA fragment may contain a portion of 1) the LOX3 gene or 2) a portion of the LOX2 gene or 3) a portion of the LOX1 gene or 4) combinations of portions of the LOX1, LOX2, or LOX3 genes. Embodiments of the present invention include recombinant DNA fragments that contain portions of 1) the LOX3 gene or 2) portions of the LOX3 gene and portions of the LOX2 gene and in either case, about 30, no more than 32 contiguous nucleotides identical to those of LOX1. Thus, lack of LOX1 activity indicates that all three known soybean seed lipoxygenases (LOX1, LOX2, and LOX3) have been suppressed. Assays may be conducted on soybean somatic embryo cultures and seeds to determine suppression of LOX1, LOX2, and LOX3.

Transgenic soybean plants having reduced levels of seed lipoxygenases and reduced levels of fatty acid desaturase, beta-amyrin synthase, oxidosqualene cyclase, isoflavone synthase, chalcone synthase, flavanone 3-hydroxylase, hydroperoxide lyase, and combinations thereof, are also prepared and assayed for suppression of each enzyme of interest. In each case, transformed tissue capable of growing in selective media is assayed for LOX1 activity as explained above, and for activity of the enzymes of the lipid oxidation pathway, fatty acid desaturation pathway, phenylpropanoid pathway and triterpenoid pathway using one or more of the following methods. The second enzyme of interest may be assayed by observing an alteration in level of a substrate upon which the enzyme of interest is known to act upon, observing an alteration in level of a product which the enzyme of interest is known to produce, determining the levels of mRNA of the enzyme of interest, assaying for the levels of activity of the enzyme of interest or of the protein itself. Assays to detect proteins may be performed by SDS-polyacrylamide gel electrophoresis using protein staining or immunological detection. Assays to detect levels of substrates or products of enzymes may be performed using gas chromatography or liquid chromatography for separation and UV or visible spectrometry or mass spectrometry for detection, or the like. Determining the levels of mRNA of the enzyme of interest may be accomplished using northern-blotting or RT-PCR techniques.

The level of suppression of lipoxygenase can be determined by either an enzyme activity assay or a western blot assay. Transformation events that yield somatic embryos that exhibit about greater than 90% reduction of lipoxygenase activity as compared to wild-type somatic embryos are considered suppressed and are of interest for regeneration into plants. Seeds from the transformed plants that exhibit about greater than 90% reduction of lipoxygenase activity or protein levels as compared to wild-type soybeans are considered suppressed.

The efficacy of suppression of fatty acid desaturases including FAD2-1, FAD2-2 and FAD3 can be determined by measuring levels fatty acids using gas chromatography. Somatic embryos that exhibit levels of oleic acid (18:1) about greater than 25% of the total fatty acids are considered suppressed in FAD2-1 and are of interest for regeneration into plants. Higher levels of 18:1 (about greater than 50%) are indicative of suppression of FAD2-2 in addition to FAD2-1. Seeds that have levels of oleic acid (18:1) about greater than 70% are considered suppressed in FAD2-1. Higher levels of 18:1 (about greater than 80%) are indicative of suppression of FAD2-2 in addition to FAD2-1. Seeds that have levels of linolenic acid (18:3) about less than 4% are considered suppressed in FAD3.

The efficacy of suppression of isoflavone synthase can be determined by measuring levels of isoflavones in soybean seeds using high performance liquid chromatography to separate the isoflavones and ultraviolet wavelength spectrophotometric detection to measure levels of isoflavones. Seeds that have levels of isoflavones about less than 20% of wild-type soybean seed levels are considered suppressed.

The efficacy of suppression of flavonol synthase can be determined by measuring levels of flavonols in soybean seeds using using high performance liquid chromatography to separate the isoflavones and mass spectrometric detection to measure levels of flavonols. Seeds that have levels of flavonols about less than 20% of wild-type soybean seed levels are considered suppressed.

The efficacy of suppression chalcone synthase can be determined by measuring levels of both isoflavones and flavonols in soybean seeds as described above. Seeds that have levels of isoflavones about less than 20% of wild-type soybean seed levels and have levels of flavonols about less than 20% of wild-type soybean seed levels are considered suppressed.

The efficacy of suppression of β-amyrin synthase can be determined by measuring levels of sapogenols in soybean seeds using high performance liquid chromatography to separate the compounds and a mass spectrometric detection to measure levels of sapogenols. Seeds that have levels of sapogenols about less than 20% of wild-type soybean seed levels are considered suppressed.

The efficacy of suppression of hydroperoxide lyase can be determined by RT-PCR. Seeds in which DNA bands are not amplified as determined by visual inspection after 35 PCR cycles are considered suppressed.

Once plants have been regenerated, and progeny plants homozygous for the transgene have been obtained, plants will have a stable phenotype that will be observed in similar seeds in later generations.

It is well understood by those skilled in the art that fragments containing sequences other than those specifically exemplified in the recombinant DNA fragments specifically mentioned above, and which have similar functions, may be used. These fragments may include any seed-specific promoter. These fragments may or may not also include any nucleotides that promote stem-loop formation. These fragments may contain a polynucleotide having a nucleotide sequence identical to any portion of the gene or genes mentioned above inserted in sense or anti-sense orientation with respect to the promoter. Finally, these fragments may or may not contain any transcription termination signal.

### EXAMPLES

All patents, patent applications, and publications cited throughout the application are incorporated by reference in their entirety.

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Preparation of cDNA Libraries and Sequencing of Entire cDNA Inserts

cDNA libraries representing mRNAs from various soybean tissues were prepared in Uni-ZAP™ XR vectors according to the manufacturer's protocol (Stratagene Cloning Systems, La Jolla, Calif.). Conversion of the Uni-ZAP™ XR libraries into plasmid libraries was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted cDNA sequences or plasmid DNA was prepared from cultured bacterial cells. Amplified insert DNAs or plasmid DNAs were sequenced in dye-primer sequencing reactions to generate partial cDNA sequences (expressed sequence tags or “ESTs”; see Adams, M. D. et al., (1991) Science 252:1651). The resulting ESTs were analyzed using a Perkin Elmer Model 377 fluorescent sequencer.

Full-insert sequence (FIS) data was generated utilizing a modified transposition protocol. Clones identified for FIS were recovered from archived glycerol stocks as single colonies, and plasmid DNAs were isolated via alkaline lysis. Isolated DNA templates were reacted with vector primed M13 forward and reverse oligonucleotides in a PCR-based sequencing reaction and loaded onto automated sequencers. Confirmation of clone identification was performed by sequence alignment to the original EST sequence from which the FIS request was made.

Confirmed templates were transposed via the Primer Island transposition kit (PE Applied Biosystems, Foster City, Calif.) which is based upon the Saccharomyces cerevisiae Ty1 transposable element (Devine and Boeke (1994) Nucleic Acids Res. 22:3765-3772). The in vitro transposition system places unique binding sites randomly throughout a population of large DNA molecules. The transposed DNA was then used to transform DH10B electro-competent cells (Gibco BRLULife Technologies, Rockville, Md.) via electroporation. The transposable element contains an additional selectable marker (named DHFR; Fling and Richards (1983) Nucleic Acids Res. 11:5147-5158), allowing for dual selection on agar plates of only those subclones containing the integrated transposon. Multiple subclones were randomly selected from each transposition reaction, plasmid DNAs were prepared via alkaline lysis, and templates were sequenced (ABI Prism dye-terminator ReadyReaction mix) outward from the transposition event site, utilizing unique primers specific to the binding sites within the transposon.

Sequence data was collected (ABI Prism Collections) and assembled using Phred/Phrap (P. Green, University of Washington, Seattle). Phred/Phrap is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies were viewed by the Consed sequence editor (D. Gordon, University of Washington, Seattle).

### Example 2

### Characterization of cDNA Clones

cDNA clones encoding soybean LOX1, LOX2, LOX3, CHS, HPL, IFS, F3H, BAM, and OSC were identified in the Du Pont proprietary EST database. The possible function of the polypeptide encoded by each cDNA was identified by conducting BLAST (Basic Local Alignment Search Tool; Altschul, S. F., et al., (1993) J. Mol. Biol. 215:403-410) searches of the ESTs against public databases. The searches were conducted for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The sequences were analyzed for similarity using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J. (1993) Nature Genetics 3:266-272) provided by the NCBI.

Characterization of cDNAs encoding soybean LOX1, LOX2, LOX3, CHS, HPL, IFS, F3H, β-amyrin synthase, or oxidosqualene cyclase follow.

A. LOX1, LOX2. and LOX3

cDNAs encoding entire soybean seed LOX1, LOX2, or LOX3 were identified in libraries prepared from 9 to 11 mm soybean developing embryos (sde4c library), from soybean embryos 19 days after flowering (se4 library), and from soybean cotyledons 7 days after germination (sgc1c library). A cDNA encoding an entire soybean LOX1 was present on clone sde4c.pk0003.c8; the nucleotide sequence of the entire cDNA insert in this clone is shown in SEQ ID NO:1. A cDNA encoding an entire soybean LOX2 was present on clone se4.pk0007.e7; the nucleotide sequence of the entire cDNA insert in this clone is shown in SEQ ID NO:2. A cDNA encoding an entire soybean LOX3 was present on clone sgs1c.pk002.g4; the nucleotide sequence of the entire cDNA insert in this clone is shown in SEQ ID NO:3.

Alignment of the nucleotide sequences from SEQ ID NOs:1, 2, and 3 with the nucleotide sequences encoding soybean LOX1 (NCBI General Identifier No. 18674; shown in SEQ ID NO:4), soybean LOX2 (NCBI General Identifier No. 170013; shown in SEQ ID NO:5), and soybean LOX3 (NCBI General Identifier No.1794171; shown in SEQ ID NO:6) indicates that the nucleotide sequences of SEQ ID NOs:1, 2, and 3 are over 99.8% identical to the corresponding published sequences encoding soybean seed lipoxygenases.

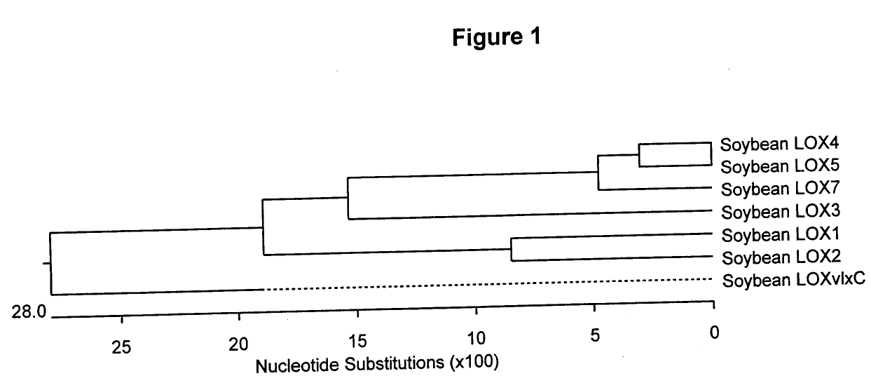

A phylogenetic tree of soybean lipoxygenases is shown in FIG. 1. This tree was assembled using the DNA Star suite and the amino acid sequences for known soybean lipoxygenases. This tree clearly shows that soybean LOX1 and LOX2 are closely related, while the soybean LOX3 is a more distant relative. The longest stretch of identical nucleotides shared by all three soybean seed lipoxygenase genes contains 32 contiguous nucleotides (shown in SEQ ID NO:7) and the longest stretch of identical nucleotides shared by LOX1 and LOX2 contains 50 contiguous nucleotides (shown in SEQ ID NO:8).

B. Chalcone Synthase

A soybean cDNA encoding an entire chalcone synthase (CHS) was identified in a library prepared from soybean roots (sr1 library). The cDNA insert in clone sr1.pk0097.b11 encodes an entire soybean chalcone synthase and its nucleotide sequence is shown in SEQ ID NO:9.

Alignment of the nucleotide sequence from SEQ ID NO:9 with the nucleotide sequence of soybean chalcone synthase found in NCBI General Identifier No. 169936 indicates that the nucleotide sequence of SEQ ID NO:9 is over 99.9% identical to the corresponding published sequences encoding soybean chalcone synthase 7.

C. Hydroneroxide Lyases (HPLS)

The information included in instant Example 2C is contained in U.S. patent Publication No. 20040010822, published 15 Jan. 2004. Soybean cDNAs encoding entire hydroperoxide lyases were identified in libraries prepared from developing pods (sdp3c and sdp4c libraries). The BLASTX search using the EST sequences from clones listed in Table 2 revealed similarity of the polypeptides encoded by the cDNAs to hydroperoxide lyases from Medicago sativa, Cucumis sativus, or Cucumis melo (NCBI General Identifier Nos. 5830467, 7576889, and 14134199, respectively). Shown in Table 2 are the BLASTP results obtained for the amino acid sequences of the entire hydroperoxide lyases encoded by the entire cDNA inserts comprising the indicated cDNA clones.

TABLE 2
BLAST Results for Sequences Encoding Polypeptides
Homologous to Hydroperoxide Lyase

| | NCBI General | |
|---|---|---|
| Clone | Identifier No. | BLAST pLog Score |
| sdp3c.pk017.j17:fis | 5830467 | >180.00 |
| sdp4c.pk015.e22:fis | 7576889 | 172.00 |
| sgs4c.pk002.f8:fis | 14134199 | 171.00 |

The nucleotide sequence corresponding to the entire cDNA insert in clone sdp3c.pk017.j17 is shown in SEQ ID NO:10; the amino acid sequence corresponding to the translation of nucleotides 49 through 1470 is shown in SEQ ID NO:11 (nucleotides 1471-1473 encode a stop). The nucleotide sequence of the entire cDNA insert in clone sdp4c.pk015.e22 is shown in SEQ ID NO:12; the amino acid sequence corresponding to the translation of nucleotides 44 through 1477 is shown in SEQ ID NO:13 (nucleotides 1478-1480 encode a stop). The nucleotide sequence of the entire cDNA insert in clone sgs4c.pk002.f8 is shown in SEQ ID NO:14; the amino acid sequence corresponding to the translation of nucleotides 52 through 1512 is shown in SEQ ID NO:15 (nucleotides 1513-1515 encode a stop).

The data in Table 3 presents the results obtained for the calculation of the percent identity of the amino acid sequences set forth in SEQ ID NOs:11, 13, and 15, with the hydroperoxide lyase sequences from Arabidopsis thaliana (NCBI General Identifier No. 11357336), Medicago sativa (NCBI General Identifier No. 5830467), Cucumis sativus (NCBI General Identifier No. 7576889), and Cucumis melo (NCBI General Identifier No. 14134199).

TABLE 3
Percent Identity of Deduced Amino Acid Sequences
Homologous to Hydroperoxide Lyases

| | | Percent Identity to | | | |
|---|---|---|---|---|---|
| Clone | SEQ ID NO. | 11357336 | 5830467 | 7576889 | 14134199 |
| sdp3c.pk017.j17:fis | 11 | 61.0 | 77.2 | 33.5 | 33.8 |
| sdp4c.pk015.e22:fis | 13 | 37.7 | 34.7 | 59.0 | 59.0 |
| sgs4c.pk002.f8:fis | 15 | 38.6 | 34.0 | 57.3 | 58.4 |

Sequence alignments and percent identity calculations were performed using the Megalign program of the LASERGENE bioinformatics computing suite (DNASTAR Inc., Madison, Wis.). Multiple alignment of the sequences was performed using the Clustal V method of alignment (Higgins and Sharp (1989) CABIOS. 5:151-153) with the default parameters (GAP PENALTY=10, GAP LENGTH PENALTY=10). Default parameters for pairwise alignments using the Clustal method were KTUPLE 1, GAP PENALTY=3, WINDOW=5 and DIAGONALS SAVED=5. Sequence alignments and BLAST scores and probabilities indicate that the nucleic acid fragments comprising the instant cDNA clones encode entire soybean hydroperoxide lyases.

Further evidence that the proteins encoded by the sgs4c.pk002.f8, sdp4c.pk015.e22, and sdp3c.pk017.j17 cDNAs are hydroperoxide lyases was provided by enzyme assays of the proteins obtained by expression of the clones in E. coli. The polynucleotides encoding HPL from clones sgs4c.pk002.f8, sdp4c.pk015.e22, and sdp3c.pk017.j17 were amplified using PCR and cloned into vector pET30 Xa/LIC (Novagen, Madison, Wis.) to create plasmids HPL1, HPL2, and HPL3, respectively. Amplification was performed using a GeneAmp PCR System 9700 (Applied Biosystems, Foster City, Calif.). After amplification each PCR product was gel-purified using the QIAquick Gel Extraction Kit (Qiagen, Valencia, Calif.), and used in cloning according to Novagen's Xa/ LIC Vector Kit protocol to produce plasmids HPL1, HPL2, and HPL3.

To express the HPL proteins in vitro each HPL plasmid was transformed into BL21 Star (DE3) competent cells (Invitrogen, Carlsbad, Calif.) according to the manufacturer's protocol. Cells containing HPL1 or HPL2 were each inoculated into 25 mL LB liquid medium containing 50 μg/mL Kanamycin (LB-Kan 50) and grown in a shaking incubator for 18 hours at 37° C. A 10 mL aliquot was removed from each culture, inoculated into flasks containing 1 L LB-Kan 50, and grown in a shaking incubator for 2.5 hours at 37° C. Protein expression was induced by the addition of isopropylthio-β-galactoside (IPTG; Invitrogen) to a 1 mM final concentration. Following induction the cultures were allowed to grow while shaking for 2 hours at 200 rpm and 37° C. The cells were then collected by centrifugation (10000×g, 15 minutes) and stored at −80° C. until needed.

An additional step was carried out with plasmid HPL3. BL21 Star (DE3) cells containing the HPL3 plasmid were made competent as described in Maniatis and plasmid pGroESL (Goloubinoff, P., et al. (1989) Nature 337:44-47) was tranformed into them. Cells containing HPL3 and pGroESL were inoculated into 25 mL LB-Kan 50 and grown in a shaking incubator for 18 hours at 37° C. A 10 mL aliquot was removed from this culture, inoculated into a 1 L LB-Kan 50, and grown in a shaking incubator for 24 hours at 18° C. Protein expression was induced by the addition of IPTG to a 1 mM final concentration. Following induction the cultures were allowed to grow while shaking for 2.5 hours at 150 rpm and 18° C. The cells were then collected by centrifugation (10000×g, 20 minutes) and stored at −80° C. until needed.

Protein Purification

Cell pellets were resuspended in 8 mL BugBuster® Protein Extraction Reagent, and 8 μL Benzonase® Nuclease was added (both from Novagen). Resuspended cells were incubated shaking at 100 rpm for 20 minutes at 25° C. The soluble protein fraction was separated by centrifugation at 16000×g for 20 minutes and stored at room temperature until needed.

Purified HPL proteins were obtained using Novagen's His-Bind® Resin and Buffer Kit at 4° C. The supernantant of each HPL cell extract was put through a 1 mL resin bed volume and soluble HPL protein was eluted with 6 mL of Novagen's Elution Buffer. Two and one half mL of the column eluate was applied to a PD-10 desalting column (Amersham BioSciences, Piscataway, N.J.) previously equilibrated with 50 mM HEPES, pH 7.5, 10% glycerol. Elution of the purified HPL proteins from the PD-10 columns was accomplished using 3.5 mL of 50 mM HEPES, pH 7.5, 10% glycerol. Protein extracts were maintained on ice or in the cold room during purification. Protein concentrations were determined using the Bio-Rad protein assay and BSA as a protein standard (Hercules, Calif.).

Hydroperoxide Lyase Activity Assays

Hydroperoxide lyase activity assays were performed at room temperature essentially as described by Noordermeer et al. ((2000) Eur. J. Biochem. 267:2473-2482). Ten μL of substrate (13(S)-Hydroperoxy-(9Z,11E)-octadecadienoic acid (H9271, Sigma, St. Louis, Mo.) were added to 980 μL of 50 mM potassium phosphate buffer, pH 6.0 and the assay started by adding 10 μL of 10 μmol/μL enzyme extract. Cleavage of 13(S)-Hydroperoxy-(9Z,11E)-octadecadienoic acid was monitored by following the decrease in absorbance at 234 nm using a Cary 100 Bio UV-visible spectrophotometer (Varian, Walnut Creek, Calif.).

Detection of activity using protein purified from HPL3 plasmid (from clone sdp3c.pk017.j17) required 100 μL of extract.

Table 4 presents the activity (in μmol-min-1-mg protein-1) obtained for the purified protein extracts from plasmids HPL1, HPL2, and HPL3 and the source of the DNA.

TABLE 4
Hydroperoxide Lyase Activity of Expressed, Purified Proteins

| | | Activity |
|---|---|---|
| Source | Plasmid | μmol · min⁻¹ · mg protein⁻¹ |
| sgs4c.pk002.f8 | HPL1 | 6.28 |
| sdp4c.pk015.e22 | HPL2 | 5.33 |
| sdp3c.pk017.j17 | HPL3 | 0.05 |

Similar results were obtained using substrate prepared according to Elshof M. B. W. et al ((1996) Recl. des Trav. Chim. Pays-Bas. 115:499-504). In this case, the substrate was a mixture of the 9 and 13 isomers due to limiting oxygen during lipoxygenase biocatalysis of linoleic acid. The fact that similar results are obtained with both assays suggests that the enzymes are capable of processing both 9-hydroperoxides and 13-hydroperoxides. The results indicate that all three polypeptides have hydroperoxide lyase activity.

D. Isoflavone Synthase (IFS)

A soybean cDNA encoding an entire isoflavone synthase (IFS) was identified by analysis of the Du Pont proprietary database and has been described in PCT publication No. WO00/44909 published 03 Aug. 2000. This cDNA is from a library prepared from soybean (Glycine max L.) seeds 4 hours after germination and the sequence of the entire cDNA insert in clone sgs1c.pk006.o20 encoding an entire soybean isoflavone synthase is shown in SEQ ID NO:16.

E. Flavanone 3-Hydroxylase (F3H)

A soybean cDNA clone encoding an entire F3H was identified by analysis of DNA sequences in the Du Pont proprietary database and has been described in U.S. Pat. No. 6,570,064. This cDNA is from a library prepared from soybean (Glycine max L.) immature flowers and the sequence of the entire cDNA insert in clone sfl1.pk0040.g11 encoding an entire F3H and is shown in SEQ ID NO:17.

F. Oxidosqualene Cyclases

Soybean cDNA clones encoding entire oxidosqualene cyclases have been identified by analysis of the Du Pont proprietary database and have been described in PCT publication No. WO01/66773, published 13 Sep. 2001. Oxidosqualene cyclases were identified in clones from libraries derived from soybean (Glycine max L., 9151) sprayed with Authority herbicide (sah1c library) and soybean (Glycine max L., Bell) 8-day-old root inoculated with eggs of Cyst Nematode Race 14 for 4 days (src3c library). The entire cDNA inserts in clones sah1c.pk002.n23 and src3c.pk024.m11 were identified as encoding entire oxidosqualene cyclases and the entire cDNA insert in clone src3c.pk024.m11 was named a β-amyrin synthase due to its demonstrated ability of producing β-amyrin. The nucleotide sequence of the entire cDNA insert in clone src3c.pk024.m11 is shown in SEQ ID NO:18. The nucleotide sequence of the entire cDNA insert in clone sah1c.pk002.n23 is shown in SEQ ID NO:19.

### Example 3

### Preparation of Recombinant DNA Fragments for Suppression of Gene Expression in Seeds of Transformed Soybean

Recombinant DNA fragments were prepared to be used in transformation of soybean for suppression of gene expression of seed lipoxygenases (LOX1, LOX2, and LOX3), fatty acid desaturase (FAD2-1), chalcone synthase (CHS), isoflavone synthase (IFS), flavanone 3-hydroxylase (F3H), hydroperoxide lyase (HPL), oxidosqualene cyclase (OSC), and β-amyrin synthase (BAM). Recombinant DNA fragments expressing proteins that would be useful in identifying transformed tissue were also prepared. These latter proteins are also referred to as selectable markers. A description of the construction of the recombinant DNA fragments follows.

A. Recombinant DNA Fragment 1025

Recombinant DNA fragment 1025 was constructed to test whether the polynucleotide encoding a soybean seed lipoxygenase 3 provides enough sequence similarity to lead to silencing of all three seed lipoxygenase genes. Recombinant DNA fragment 1025 (the sequence of which is shown in SEQ ID NO:20) comprises in 5′ to 3′ orientation:
a) about 2088 nucleotides of the Kti3 promoter;
b) a 74-nucleotide synthetic sequence;
c) a unique Eco RI restriction endonuclease site where a 2226 nucleotide DNA fragment from the soybean seed lipoxygenase 3 has been inserted;
d) an inverted repeat of the nucleotides in b), and
e) about 202 nucleotides of the Kti3 transcription terminator.

The nucleotides in the synthetic repeats of b) and d) promote formation of a stem in a stem-loop structure. Transgenic organisms prepared with this type of recombinant DNA fragments have been shown to have reduced levels of the protein encoded by the nucleotide fragment forming the loop as described in PCT Publication WO 02/00904, published 03 Jan. 2002.

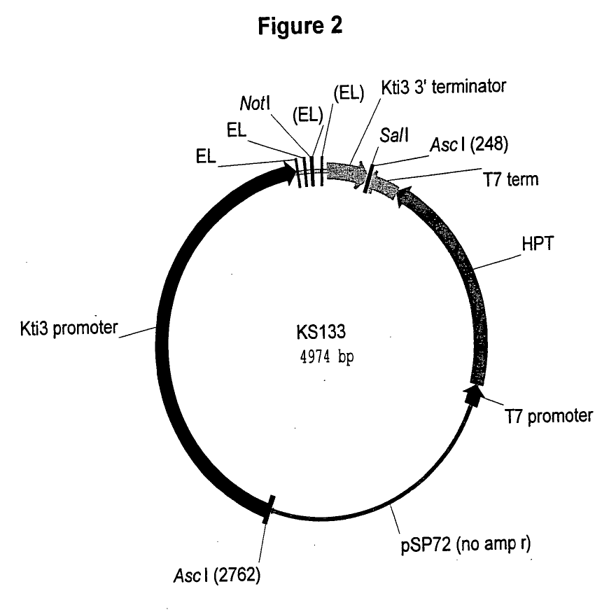

To construct recombinant DNA fragment 1025, a seed-specific gene expression-silencing cassette was obtained from vector pKS133 (whose map is shown in FIG. 2 and sequence shown in SEQ ID NO:86) and modified. Vector pKS133 has been described in PCT Publication WO 02/00904, published 03 Jan. 2002, and is derived from the commercially available vector pSP72 (Promega, Madison, Wis.). The seed-specific gene expression-silencing cassette of pKS133 comprises:
a) about 2088 nucleotides of the Kti3 promoter,
b) 74-nucleotide synthetic sequence,
c) a unique Not I restriction endonuclease site,
d) an inverted repeat of the nucleotides in b), and
e) about 202 nucleotides of the Kti3 transcription terminator.

The gene encoding Kti3 has been described (Jofuku and Goldberg (1989) Plant Cell 1:1079-1093). The 74-nucleotide synthetic sequences of b) and d) promote formation of a stem structure. Insertion of a nucleotide fragment from a desired gene in the unique Not I site has been shown to result in suppression of the desired gene as described in PCT Publication WO 02/00904, published 03 Jan. 2002. The nucleotide sequence of this seed-specific gene expression-silencing cassette from pKS133 is shown in SEQ ID NO:21.

To generate recombinant DNA fragment 1025 the seed-specific gene expression-silencing cassette from pKS133 was modified by replacing the unique Not I site with a unique Eco RI site and inserting into this unique site a polynucleotide from a soybean seed lipoxygenase 3 gene. The unique Eco RI site was generated by inserting into the Not I site of pKS133, by DNA ligation, a self-annealing oligonucleotide linker (shown in SEQ ID NO:22). A 2226 nucleotide DNA fragment from the soybean seed lipoxygenase 3 was obtained by digesting with Eco RI the cDNA insert in clone sgs1c.pk002.g4, and was then inserted into the Eco RI site of the gene expression-silencing cassette. The seed-specific gene expression-silencing cassette from recombinant DNA fragment 1025 forms a “stem-loop” structure where the 2226 nucleotide fragment from LOX3 forms the loop and the 74 nucleotide synthetic sequences form the stem. For use in plant transformation experiments recombinant DNA fragment 1025 was obtained by digesting the plasmid with restriction endonuclease Asc I and isolating recombinant DNA fragment 1025, having 4690 bp, by agarose gel electrophoresis.

B. Recombinant DNA Fragment 1028

Recombinant DNA fragment 1028 was constructed to provide additional sequence similarity to the LOX 1 and LOX2 genes in order to more efficiently suppress expression of all three soybean seed lipoxygenase genes. Recombinant DNA fragment 1028 (the sequence of which is shown in SEQ ID NO:23) comprises in 5′ to 3′ orientation:
a) about 2088 nucleotides of the Kti3 promoter;
b) 74-nucleotide synthetic sequence,
c) a unique Eco RI restriction endonuclease site containing a 1364-nucleotide DNA fragment from the soybean LOX3 gene and a 523-nucleotide DNA fragment from the soybean LOX2 gene;
d) an inverted repeat of the nucleotides in b), and
e) about 202 nucleotides of the Kti3 transcription terminator.

The nucleotide synthetic sequences in b) and d) promote formation of a stem in a stem-loop structure where the nucleotide fragment of c) forms the loop. This stem-loop structure has been shown to result in suppression of the gene having similarity to the nucleotide fragment forming the loop as described in PCT Publication WO 02/00904, published 03 Jan. 2002.

To construct recombinant DNA fragment 1028 an 862-nucleotide fragment from the soybean LOX3 gene in recombinant DNA fragment 1025 was removed by digestion with Pst I and Sph I. This fragment was replaced with a 523 nucleotide soybean LOX2 DNA fragment obtained by digestion of clone se4.pk0007.e7 with Pst I and Sph I. This 523 nucleotide soybean LOX2 DNA fragment contains 3 regions with 32 or more contiguous nucleotides that are identical between soybean LOX1 and soybean LOX2 genes; the longest common sequence is 50 contiguous nucleotides (shown in SEQ ID NO:8). For use in plant transformation experiments recombinant DNA fragment 1028 was obtained by digesting the plasmid with restriction endonuclease Asc I and isolating the 4351 bp recombinant DNA fragment 1028 by agarose gel electrophoresis.

C. ALS Selectable Marker Recombinant DNA Fragment

A recombinant DNA fragment comprising a constitutive promoter directing expression of a mutant soybean acetolactate synthase (ALS) gene followed by the soybean ALS 3′ transcription terminator was used as a selectable marker for soybean transformation. The constitutive promoter used is a 1.3-Kb DNA fragment that functions as the promoter for a soybean S-adenosylmethionine synthase (SAMS) gene and is described in PCT publication No. WO 00/37662 published 29 Jun. 2000. The nucleotide sequence of this recombinant DNA fragment used as a selectable marker is shown in SEQ ID NO:24. The mutant soybean ALS gene encodes an enzyme that is resistant to inhibitors of ALS, such as sulfonylurea herbicides. The deduced amino acid sequence of the mutant soybean ALS present in the recombinant DNA fragment used as a selectable marker is shown in SEQ ID NO:25.

Mutant plant ALS genes encoding enzymes resistant to sulfonylurea herbicides are described in U.S. Pat. No. 5,013,659. One such mutant is the tobacco SURB-Hra gene, which encodes a herbicide-resistant ALS with two substitutions in the amino acid sequence of the protein. This tobacco herbicide-resistant ALS contains alanine instead of proline at position 191 in the conserved “subsequence B” (shown in SEQ ID NO:26) and leucine instead of tryptophan at position 568 in the conserved “subsequence F” (shown in SEQ ID NO:27) (U.S. Pat. No. 5,013,659; Lee et al. (1988) EMBO J 7:1241-1248).

The ALS selectable marker recombinant DNA fragment was constructed using a polynucleotide for a soybean ALS to which the two Hra-like mutations were introduced by site directed mutagenesis. Thus, this recombinant DNA fragment will translate to a soybean ALS having alanine instead of proline at position 183 and leucine instead of tryptophan at position 560.

In addition, during construction of SAMS promoter-mutant ALS expression cassette, the coding region of the soybean ALS gene was extended at the 5′-end by five additional codons, resulting in five amino acids (M-P-H-N-T; shown in SEQ ID NO:28), added to the amino-terminus of the ALS protein. These extra amino acids are adjacent to and presumably removed with the transit peptide during targeting of the mutant soybean ALS protein to the plastid. A DNA fragment comprising a polynucleotide encoding the soybean ALS was digested with Kpn I, blunt ended with T4 DNA polymerase, digested with Sal I, and inserted into a plasmid containing the SAMS promoter which had been previously digested with Nco I and blunt ended by filling-in with Klenow DNA polymerase. For use in plant transformation experiments the ALS selectable marker recombinant DNA fragment was obtained by digesting the plasmid with restriction endonuclease Asc I and isolating the 3964 bp ALS selectable marker recombinant DNA fragment by agarose gel electrophoresis.

D. Recombinant DNA Fragment 1029

Recombinant DNA fragment 1029 contains a seed lipoxygenase gene expression silencing cassette and a selectable marker gene used for soybean transformation. The nucleotide sequence of recombinant DNA fragment 1029 is shown in SEQ ID NO:29. This recombinant DNA fragment contains the lipoxygenase gene expression-silencing cassette from recombinant DNA fragment 1028 (described in Example 3B, above) and the ALS selectable marker recombinant DNA fragment described in Example 3C, above.

Recombinant DNA fragment 1029 was prepared by removing the ALS selectable marker recombinant DNA fragment from its plasmid by digesting with the restriction endonuclease Xho I and inserting this fragment into a Sal I-digested plasmid carrying recombinant DNA fragment 1028. For transformation, the DNA fragment containing both the lipoxygenase gene expression-silencing cassette and the ALS selectable marker cassette was obtained by digesting the plasmid with restriction endonuclease Asc I and isolating the recombinant DNA fragment 1029 having 8336 bp, by agarose gel electrophoresis.

E. Recombinant DNA Fragment KS136

Recombinant DNA fragment KS136 contains a fatty acid desaturase (FAD2) seed-specific gene expression silencing cassette. Recombinant DNA fragment KS136 comprises in 5′ to 3′ orientation:
a) about 2088 nucleotides of the Kti3 promoter,
b) 74-nucleotide synthetic sequence,
c) a unique Not I restriction endonuclease site where an approximately 600 nucleotide fragment of the fatty acid desaturase 2 (FAD2) gene has been inserted,
d) an inverted repeat of the nucleotides in b), and
e) about 202 nucleotides of the Kti3 transcription terminator.

The nucleotide sequence of recombinant DNA fragment KS136 is shown in SEQ ID NO:30.

The seed-specific gene expression-silencing cassette of recombinant DNA fragment KS136 is derived from the vector pKS133 described in Example 3A above. An approximately 600 nucleotide fragment of the FAD2-1 gene was inserted into the Not I site of pKS133 to form KS136. The nucleotide sequence of the approximately 600 nucleotide fragment of the FAD2-1 gene is shown in SEQ ID NO:31. For use in plant transformation experiments recombinant DNA fragment KS136 was obtained by digesting the plasmid with restriction endonuclease Asc I and isolating recombinant DNA fragment KS136, having 3072 bp, by agarose gel electrophoresis.

F. Recombinant DNA Fragment PHP19853A

Recombinant DNA fragment PHP19853A includes a gene expression silencing cassette designed to silence seed lipoxygenases and FAD2-1 linked in a head to head configuration to the ALS selectable marker recombinant DNA fragment of Example 3C above. The mRNA transcripts of the gene expression silencing cassette and the selectable marker cassette run in opposite orientations. The nucleotide sequence of recombinant DNA fragment PHP19853A is shown in SEQ ID NO:32. Recombinant DNA fragment PHP19853A comprises in 5′ to 3′ orientation:
a) the complementary strand of the ALS selectable marker recombinant DNA fragment of Example 3C above,
b) about 2088 nucleotides of the Kti3 promoter,
c) 74-nucleotide synthetic sequence,
d) a unique Not I restriction endonuclease site containing an approximately 2480 polynucleotide fragment comprising about 1880 nucleotides from recombinant DNA fragment 1028 which includes about 1360 nucleotides from the soybean seed LOX3 gene and 520 nucleotides from the soybean seed LOX2 gene, and 600 nucleotides from the FAD2-1 gene,
e) an inverted repeat of the nucleotides in c), and
f) about 202 nucleotides of the Kti3 transcription terminator

Recombinant DNA fragment PHP19853A was constructed as follows. An approximately 1.9 kb DNA fragment was obtained by PCR amplification using primers TW108 (the nucleotide sequence of which is shown in SEQ ID NO:34) and TW109 (the nucleotide sequence of which is shown in SEQ ID NO:35) and using recombinant DNA fragment 1028 as template.

TW108:
5′-CGATGCGGCCGCAATTCCTGGAGCATTTTATATC-3′
TW109:
5′-CACTCGTGAGCAATCACTCACCTCTGAAAGTTAATCCTTC-3′

An approximately 0.6 kb DNA fragment was obtained by PCR amplification using primers TW10 (the nucleotide sequence of which is shown in SEQ ID NO:36) and KS99 (the nucleotide sequence of which is shown in SEQ ID NO:37) and using recombinant DNA fragment KS136 (from Example 3E) as template.

TW110:
5′-GAAGGATTAACTTTCAGAGGTGAGTGATTGCTCACGAGTG-3′
KS99:
5′GAATTCGCGGCCGCTTAATCTCTGTCCATAGTT-3′

The 1.9 kb fragment and 0.6 kb fragment were mixed and used as template for a PCR amplification with primers TW108 and KS99 to yield an approximately 2480 bp fragment that was cloned into the commercially available plasmid pCR2.1 using the TOPO TA Cloning Kit (Invitrogen). After digestion with Not I the 2480 bp fragment having the nucleotide sequence shown in SEQ ID NO:33 was isolated. To prepare the plasmid containing recombinant DNA fragment PHP19853A, plasmid pKS210 containing recombinant DNA fragment PHP19104A (see Example 3K below) was digested with Not I to remove the portion containing LOX3, LOX2, BAM, and OSC nucleotides and the 2480 bp Not I fragment (having the nucleotide sequence shown in SEQ ID NO:33) was ligated into the Not I site.

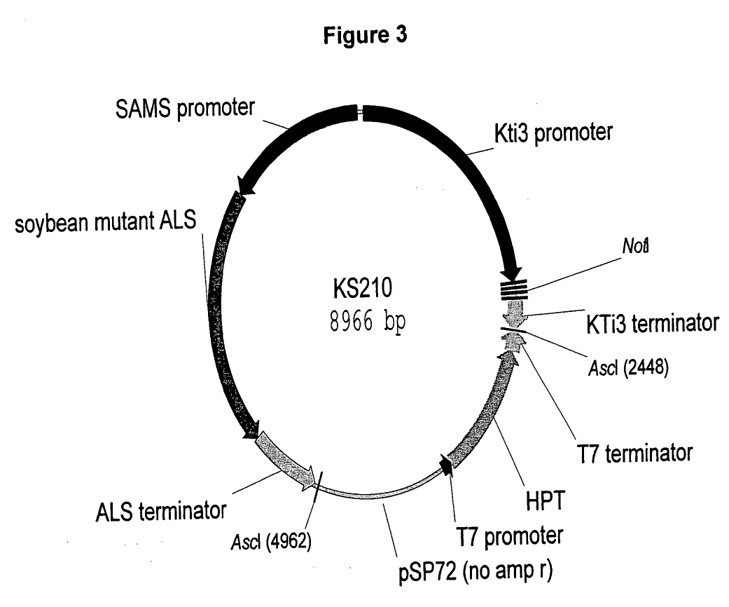

Plasmid pKS210 is derived from the commercially available cloning vector pSP72 (Promega). The beta lactamase coding region has been replaced by a hygromycin phosphotransferase gene for use as a selectable marker in E. coli. In addition, a gene expression silencing cassette linked in a head to head configuration to the ALS selectable marker recombinant DNA fragment of Example 3C has been added. The gene expression silencing cassette in plasmid pKS210 comprises the KTi3 promoter, a 74 nucleotide synthetic sequence, a unique Not I restriction endonuclease site, an inverted repeat of the 74 nucleotide synthetic sequence, and the Kti3 terminator region. A map of plasmid pKS210 is shown in FIG. 3 and its nucleotide sequence is disclosed in SEQ ID NO:87.

For use in plant transformation experiments the 8948 bp recombinant DNA fragment PHP19853A was removed from its cloning plasmid using restriction endonuclease Asc I and was separated from the remaining plasmid DNA by agarose gel electrophoresis.

G. Recombinant DNA Fragment PHP19112A

Recombinant DNA fragment PHP19112A contains a gene expression silencing cassette designed to silence expression of seed lipoxygenases (LOX) and chalcone synthase (CHS) linked in a head to head configuration to the ALS selectable marker recombinant DNA fragment of Example 3C above. The nucleotide sequence of recombinant DNA fragment PHP19112A is shown in SEQ ID NO:38. The portion of CHS gene was obtained from a soybean cDNA encoding an entire CHS that was identified in the DuPont proprietary database as explained in Example 2B above. Recombinant DNA fragment PHP19112A contains in 5′ to 3′ orientation:
a) the complementary strand of the ALS selectable marker recombinant DNA fragment of Example 3C above,
b) about 2088 nucleotides of the Kti3 promoter,
c) 74-nucleotide synthetic sequence,
d) a unique Not I restriction endonuclease site containing an approximately 2250 polynucleotide fragment comprising about 1140 nucleotides from the soybean LOX3 gene, 520 nucleotides from the soybean LOX2 gene, and 586 nucleotides from a soybean CHS gene,
e) an inverted repeat of the nucleotides in c), and
f) about 202 nucleotides of the Kti3 transcription terminator.

The sequence of the approximately 2250 polynucleotide fragment is shown in SEQ ID NO:39. The 2250 polynucleotide fragment comprising about 1140 nucleotides from the soybean LOX3 gene, 520 nucleotides from the soybean LOX2 gene, and 586 nucleotides from a soybean CHS gene was constructed by PCR amplification as follows.

An approximately 1.7 kb DNA fragment was obtained by PCR amplification using primers BM1 (the nucleotide sequence of which is shown in SEQ ID NO:40) and BM2 (the nucleotide sequence of which is shown in SEQ ID NO:41) and using recombinant DNA fragment 1028 as template.

BM1: 5′-GCGGCCGCAATTCCTGGAGCATTTTATATC-3′
BM2: 5′-CTACGCTAAGCGGCCGCATGCCTTGACAAGATCTC-3′

An approximately 0.6 kb DNA fragment was obtained by PCR amplification using primers BM3 (the nucleotide sequence of which is shown in SEQ ID NO:42) and BM4 (the nucleotide sequence of which is shown in SEQ ID NO:43) and using clone sr1.pk0097.b11 (which, as mentioned in Example 2B, encodes an entire CHS) as template.

BM3: 5′-CTTGTCAAGGCATGCGGCCGCTTAGCGTAGCTGAG-3′
BM4: 5′-GCGGCCGCGTGACTGCAGTGATCTCAGAGC-3′

The 1.7 kb fragment and 0.6 kb fragment were mixed and used as template for a PCR amplification with BM1 and BM4 as primers to yield an approximately 2250 bp fragment that was cloned into the commercially available plasmid pCR2.1 using the TOPO TA Cloning Kit (Invitrogen). After digestion with Not I the 2250 bp fragment having the nucleotide sequence shown in SEQ ID NO:39 was ligated into the Not I site of plasmid pKS210, described in Example 3F above.

For use in plant transformation experiments the 8716 bp recombinant DNA fragment PHP19112A was removed from its cloning plasmid using restriction endonuclease Asc I and was separated from the remaining plasmid DNA by agarose gel electrophoresis.

H. Recombinant DNA Fragment PHP19113A

Recombinant DNA fragment PHP19113A contains a gene expression silencing cassette designed to silence soybean seed lipoxygenases (LOX) and isoflavone synthase (IFS) linked in a head to head configuration to the ALS selectable marker recombinant DNA fragment of Example 3C above. The nucleotide sequence of recombinant DNA fragment PHP19113A is shown in SEQ ID NO:44. The portion of IFS used to prepare recombinant DNA fragment PHP19113A was obtained from a soybean cDNA encoding an entire IFS which was identified by analysis of DNA sequences in the Du Pont proprietary database as explained in Example 2D. Recombinant DNA fragment PHP19113A contains in 5′ to 3′ orientation:
a) the complementary strand of the ALS selectable marker recombinant DNA fragment of Example 3C above,
b) about 2088 nucleotides of the Kti3 promoter,
c) 74-nucleotide synthetic sequence,
d) a unique Not I restriction endonuclease site containing an approximately 2440 polynucleotide fragment comprising about 1140 nucleotides from the soybean LOX3 gene and 520 nucleotides from the soybean LOX2 gene, and 786 nucleotides from a soybean IFS gene,
e) an inverted repeat of the nucleotides in c), and
f) about 202 nucleotides of the Kti3 transcription terminator.

The nucleotide sequence of the entire cDNA insert in soybean clone sgs1c.pk006.o20 encodes an entire IFS and is shown in SEQ ID NO:16. The sequence of the approximately 2440 polynucleotide fragment is shown in SEQ ID NO:45. The 2440 polynucleotide fragment comprising about 1140 nucleotides from the soybean LOX3 gene and 520 nucleotides from the soybean LOX2 gene, and 786 nucleotides from a soybean IFS gene was constructed by PCR amplification as follows.

An approximately 1.7 kb DNA fragment was obtained by PCR amplification using primers BM1 (the nucleotide sequence of which is shown in SEQ ID NO:40) and BM8 (the nucleotide sequence of which is shown in SEQ ID NO:46), and recombinant DNA fragment 1028 as template.

BM1: 5′-GCGGCCGCAATTCCTGGAGCATTTTATATC-3′
BM8: 5′-CTCAACAACTTCTCCCTTGACAAGATCTCTATCAC-3′

An approximately 0.8 kb DNA fragment was obtained by PCR amplification using primers BM9 (the nucleotide sequence of which is shown in SEQ ID NO:47) and BM10 (the nucleotide sequence of which is shown in SEQ ID NO:48), and the cDNA insert from clone sgs1c.pk006.o20 as template.

| | |
|---|---|
| BM9: | 5′-AGAGATCTTGTCAAGGGAGAAGTTGTTGAGGGCGAG-3′ |
| BM10: | 5′-GCGGCCGCTTAAGAAAGGAGTTTAGATGCAAC-3′ |

The 1.7 kb fragment and 0.8 kb fragment were mixed and used as template for a PCR amplification with BM1 and BM10 as primers to yield an approximately 2440 bp fragment that was cloned into the commercially available plasmid pCR2.1 using the TOPO TA Cloning Kit (Invitrogen). After digestion with Not I the 2440 bp fragment having the nucleotide sequence shown in SEQ ID NO:45 was ligated into the Not I site of plasmid pKS210, described in Example 3F above.

For use in plant transformation experiments the 8906 bp recombinant DNA fragment PHP19113A was removed from its cloning plasmid using restriction endonuclease Asc I and was separated from the remaining plasmid DNA by agarose gel electrophoresis.

I. Recombinant DNA Fragment PHP19027A

Recombinant DNA fragment PHP19027A contains a gene expression silencing cassette designed to silence seed lipoxygenases and flavanone 3-hydroxylase (F3H) linked in a head to head configuration to the ALS selectable marker recombinant DNA fragment described in Example 3C above. The nucleotide sequence of recombinant DNA fragment PHP19027A is shown in SEQ ID NO:49. A soybean cDNA including an entire F3H coding sequence was identified by analysis of DNA sequences in the Du Pont proprietary database as explained in Example 2E. Recombinant DNA fragment PHP19027A contains in 5′ to 3′ orientation:
a) the complementary strand of the ALS selectable marker recombinant DNA fragment of Example 3C above,
b) about 2088 nucleotides of the Kti3 promoter,
c) 74-nucleotide synthetic sequence,
d) a unique Not I restriction endonuclease site containing an approximately 2320 polynucleotide fragment comprising about 1140 nucleotides from the soybean LOX3 gene, 520 nucleotides from the soybean LOX2 gene, and 663 nucleotides from a soybean F3H gene,
e) an inverted repeat of the nucleotides in c), and
f) about 202 nucleotides of the Kti3 transcription terminator.

The portion of the F3H gene was obtained from clone sfl1.pk0040.g11 which was identified in the Du Pont proprietary database as encoding an entire soybean F3H (see Example 2E, above) and its nucleotide sequence is shown in SEQ ID NO:17. The nucleotide sequence of the 2320 polynucleotide fragment is shown in SEQ ID NO:50. The 2320 polynucleotide fragment comprising about 1140 nucleotides from the soybean LOX3 gene, 520 nucleotides from the soybean LOX2 gene, and 663 nucleotides from a soybean F3H gene was obtained by PCR amplification as follows.

An approximately 1.7 kb DNA fragment was obtained by PCR amplification using primers BM1 (the nucleotide sequence of which is shown in SEQ ID NO:40) and BM11 (the nucleotide sequence of which is shown in SEQ ID NO:51), and recombinant DNA fragment 1028 as template.

| | |
|---|---|
| BM1: | 5′-GCGGCCGCAATTCCTGGAGCATTTTATATC-3′ |
| BM11: | 5′-GGCTGTTGGTGCCATCTTGACAAGATCTCTATCAC-3′ |

An approximately 0.8 kb DNA fragment was obtained by PCR amplification using primers BM12 (the nucleotide sequence of which is shown in SEQ ID NO:52) and BM13 (the nucleotide sequence of which is shown in SEQ ID NO:53), and a DNA fragment containing the cDNA insert from clone sfl1.pk0040.g11 as template.

BM12: 5′-AGAGATCTTGTCAAGATGGCACCAACAGCCAAGAC-3′
BM13: 5′-GCGGCCGCATCCGTGTGGCGCTTCAGGCCAAG-3′

The 1.7 kb fragment and 0.6 kb fragment were mixed and used as template for a PCR amplification with BM1 and BM13 as primers to yield an approximately 2320 bp fragment that was cloned into the commercially available plasmid pCR2.1 using the TOPO TA Cloning Kit (Invitrogen). After digestion with Not I the 2320 bp fragment having the nucleotide sequence shown in SEQ ID NO:50 was ligated into the Not I site of plasmid pKS210, described in Example 3F above.

For use in plant transformation experiments the 8783 bp recombinant DNA fragment PHP19027A was removed from its cloning plasmid using restriction endonuclease Asc I and was separated from the remaining plasmid DNA by agarose gel electrophoresis.

J. Recombinant DNA Fragment PHP19338A

Recombinant DNA fragment PHP19338A contains a gene expression silencing cassette designed to silence seed lipoxygenases (LOX) and hydroperoxide lyases (HPL) linked in a head to head configuration to the ALS selectable marker recombinant DNA fragment described in Example 3C above. The nucleotide sequence of recombinant DNA fragment PHP1 9338A is shown in SEQ ID NO:54. Soybean cDNA clones encoding entire HPLs were identified by analysis of DNA sequences in the Du Pont proprietary database as explained in Example 2C. Recombinant DNA fragment PHP19338A contains in 5′ to 3′ orientation:
a) the complementary strand of the ALS selectable marker recombinant DNA fragment of Example 3C above,
b) about 2088 nucleotides of the Kti3 promoter,
c) 74-nucleotide synthetic sequence,
d) a unique Not I restriction endonuclease site containing an approximately 3290 nucleotide fragment comprising about 1140 nucleotides from the soybean LOX3 gene, 520 nucleotides from the soybean LOX2 gene, and approximately 1626 nucleotides from the soybean HPL genes,
e) an inverted repeat of the nucleotides in c), and
f) about 202 nucleotides of the Kti3 transcription terminator.

The approximately 1626 nucleotides from the soybean HPL genes correspond to 523 nucleotides from the cDNA insert in clone sdp3c.pk017.j17, 559 nucleotides from the cDNA insert in clone sgs4c.pk002.f8, and 544 nucleotides from the cDNA insert in clone sdp4c.pk015.e22. The nucleotide sequence from the 3290 nucleotide fragment is shown in SEQ ID NO:55 and was prepared by PCR as follows.

An approximately 1.7 kb DNA fragment was obtained by PCR amplification using primers BM1 (having the nucleotide sequence shown in SEQ ID NO:40) and BM14 (having the nucleotide sequence shown in SEQ ID NO:56), and recombinant DNA fragment 1028 as template.

BM1:
5′-GCGGCCGCAATTCCTGGAGCATTTTATATC-3′
BM14:
5′-GTGCTGTGTGGTGTGGTGGTTGCATGCCTTGACAAGATCTC-3′

An approximately 0.5 kb DNA fragment was obtained by PCR amplification using primers BM15 (having the nucleotide sequence shown in SEQ ID NO:57) and BM16 (having the nucleotide sequence shown in SEQ ID NO:58), and a DNA fragment comprising the cDNA insert in clone sdp3c.pk017.j17 as template.

BM15:
5′-GAGATCTTGTCAAGGCATGCAACCACCACACCACACAGCAC-3′
BM16:
5′-GAGGAGTGACAGTGTGTCTAGGTTTGATTCTAGTTCTG-3′

An approximately 0.6 kb DNA fragment was obtained by PCR amplification using primers BM17 (having the nucleotide sequence shown in SEQ ID NO:59) and BM18 (having the nucleotide sequence shown in SEQ ID NO:60), and a DNA fragment containing the cDNA insert in clone sgs4c.pk002.f8 as template.

BM17: 5′-GACACACTGTCACTCCTCCTCCTCCCTCTCTCTTCC-3′
BM18: 5′-GTTGAAGCTGGCCTTGGTGTTTTTACTCAACTGG-3′

An approximately 0.5 kb DNA fragment was obtained by PCR amplification using primers BM19 (having the nucleotide sequence shown in SEQ ID NO:61) and BM20 (having the nucleotide sequence shown in SEQ ID NO:62), and a DNA fragment containing the cDNA insert in clone sdp4c.pk015.e22 as template.

BM19:

5′-TTGAGTAAAAACACCAAGGCCAGCTTCAACTCCTCCGTCG-3′
BM20:
5′-GCGGCCTATCCTCAGGACCTCATACACCACTGATTTGG-3′

An approximately 2.2 kb DNA fragment was obtained by PCR amplification using primers BM1 (the nucleotide sequence of which is shown in SEQ ID NO:40) and BM16 (the nucleotide sequence of which is shown in SEQ ID NO:58), and a mixture containing the 1.7 kb fragment obtained by PCR amplification of recombinant DNA fragment 1028 and the 0.5 kb fragment obtained by PCR amplification of the cDNA insert in clone sdp3c.pk017.j17. This approximately 2.2 kb fragment was cloned into the commercially available plasmid pCR2.1 using the TOPO TA Cloning Kit (Invitrogen) and was named pAB.

An approximately 1.1 kb DNA fragment was obtained by PCR amplification of the 0.6 kb fragment obtained by PCR amplification of the cDNA insert in clone sgs4c.pk002.f8 and the 0.5 kb fragment obtained by PCR amplification of the cDNA insert in clone sdp4c.pk015.e22 using primers BM17 (the nucleotide sequence of which is shown in SEQ ID NO:59) and BM20 (the nucleotide sequence of which is shown in SEQ ID NO:62). This approximately 1.1 kb DNA fragment was cloned into the commercially available plasmid pCR2.1 using the TOPO TA Cloning Kit and was named pCD.

To complete the construction of the 3290 nucleotide recombinant DNA fragment containing nucleotides from lipoxygenases and HPLs, PCR amplification was performed using primers BM21 (the nucleotide sequence of which is shown in SEQ ID NO:63) and BM22 (the nucleotide sequence of which is shown in SEQ ID NO:64) and clone pAB as template, and using primers BM23 (the nucleotide sequence of which is shown in SEQ ID NO:65) and BM24 (the nucleotide sequence of which is shown in SEQ ID NO:66) and clone as pCD as template.

BM21: 5′-GCGGCCGCAATTCCTGGAGCATTTTATATC-3′
BM22: 5′-GAGGGAGGAGGAGGAGTGACAGTGTGTC-3′
BM23: 5′-ATCAAACCTAGACACACTGTCACTCCTCC-3′
BM24: 5′-GCGGCCGCTATCCTCAGGACCTCATACACC-3′

Finally, an approximately 3.3 kb fragment was obtained by PCR amplification using primers BM21 (the nucleotide sequence of which is shown in SEQ ID NO:63) and BM24 (the nucleotide sequence of which is shown in SEQ ID NO:66) and the PCR amplification products obtained using pAB and pCD as templates. This 3.3 kb fragment was cloned into the commercially available plasmid pCR2.1 using the TOPO TA Cloning Kit. After digestion with Not I the 3.3 kb fragment was digested with Not I and ligated into the Not I site of plasmid pKS210, described in Example 3F above.

For use in plant transformation experiments the 9746 bp recombinant DNA fragment PHP19338A was removed from its cloning plasmid using restriction endonuclease Asc I and was separated from the remaining plasmid DNA by agarose gel electrophoresis.

K. Recombinant DNA Fragment PHP19104A

Recombinant DNA fragment PHP19104A contains a gene expression silencing cassette designed to suppress seed lipoxygenases (LOX), β-amyrin synthases (BAM), and oxidosqualene cyclases (OSC) linked in a head to head configuration to the ALS selectable marker recombinant DNA fragment described in Example 3C above. The nucleotide sequence of recombinant DNA fragment PHP19104A is shown in SEQ ID NO:67. Soybean cDNA clones encoding entire β-amyrin synthases and oxidosqualene cyclases were identified by analysis of DNA sequences in the Du Pont proprietary database as explained in Example 2F. Recombinant DNA fragment PHP19104A contains in 5′ to 3′ orientation:
a) the complementary strand of the ALS selectable marker recombinant DNA fragment of Example 3C above,
b) about 2088 nucleotides of the Kti3 promoter,
c) 74-nucleotide synthetic sequence,
d) a unique Not I restriction endonuclease site containing an approximately 2900 nucleotide fragment comprising about 1880 nucleotides from recombinant DNA fragment 1028 that includes fragments of the soybean LOX3 and LOX2 genes, followed by about 570 nucleotides from a cDNA insert encoding a β-amyrin synthase and about 450 nucleotides from a cDNA insert encoding an oxidosqualene cyclase,
e) an inverted repeat of the nucleotides in c), and
f) about 202 nucleotides of the Kti3 transcription terminator.

The nucleotide sequence of the 2900 recombinant DNA fragment is shown in SEQ ID NO:68. The 2900 recombinant DNA fragment comprising portions of the LOX2, LOX3, β-amyrin synthase, and oxidosqualene cyclase genes was constructed by PCR amplification as follows:

An approximately 1.9 kb DNA fragment was obtained by PCR amplification using primers BM1 (the nucleotide sequence of which is shown in SEQ ID NO:40) and BM5 (the nucleotide sequence of which is shown in SEQ ID NO:69) and using recombinant DNA fragment 1028 as template.

BM1: 5′-GCGGCCGCAATTCCTGGAGCATTTTATATC-3′
BM5: 5′-CTTCAGCCTCCACATCCTCTGAAAGTTAATCCTTCC-3′

A recombinant DNA fragment comprising a portion of a soybean β-amyrin synthase gene and a portion of an oxidosqualene cyclase gene was prepared by first amplifying sequences corresponding to both genes and then mixing the amplification products for a new amplification reaction. A portion of the cDNA insert in clone sah1c.pk002.n23 was amplified using primers BM25 (the nucleotide sequence of which is shown in SEQ ID NO:70) and BM26 (the nucleotide sequence of which is shown in SEQ ID NO:71) and a portion of the cDNA insert in clone src3c.pk0024.ml 1 was amplified using primers BM27 (the nucleotide sequence of which is shown in SEQ ID NO:72) and BM28 (the nucleotide sequence of which is shown in SEQ ID NO:73).

BM25 5′-GCGGCCGCCAACAATTTAGAAGAGGCTCGG-3′
BM26: 5′-TTCTTGGAGAAGGACCTAATGGAGGTCATG-3′
BM27: 5′-GCGGCCGCATGTGGAGGCTGAAGATAGCAG-3′
BM28: 5′-GTCATGACCTCCATTAGGTCCTTCTCCAAG-3′

The DNA fragments resulting from both amplifications were combined and used as a template for amplification using primers BM29 (the nucleotide sequence of which is shown in SEQ ID NO:74) and BM30 (the nucleotide sequence of which is shown in SEQ ID NO:75) yielding fragment AC18.

BM29: 5′-GCGGCCGCATGTGGAGGCTGAAGATAGCAG-3′
BM30: 5′-GCGGCCGCCAACAATTTAGAAGAGGCTCGG-3′

An approximately 1.0 kb DNA fragment was obtained by PCR amplification using primers BM6 (the nucleotide sequence of which is shown in SEQ ID NO:76) and BM7 (the nucleotide sequence of which is shown in SEQ ID NO:77) with AC18 as template.

BM6: 5′-GATTAACTTTCAGAGGATGTGGAGGCTGAAGATAG-3′
BM7: 5′-GCGGCCGCAACAATTTAGAAGAGGCTCGGTG-3′

The 1.0 kb fragment and 1.9 kb fragment were mixed and used as template for a PCR amplification with BM1 (the nucleotide sequence of which is shown in SEQ ID NO:40) and BM7 (the nucleotide sequence of which is shown in SEQ ID NO:77) as primers to yield an approximately 2900 bp fragment that was cloned into the commercially available plasmid pCR2.1 using the TOPO TA Cloning Kit. After digestion with Not I the 2900 bp fragment was digested with Not I and ligated into the Not I site of plasmid pKS210, described in Example 3F above.

For use in plant transformation experiments the 9358 bp recombinant DNA fragment PHP19104A was removed from its cloning plasmid using restriction endonuclease Asc I and was separated from the remaining plasmid DNA by agarose gel electrophoresis.

L. Recombinant DNA Fragment PHP19962A

Recombinant DNA fragment PHP19962A contains a gene expression silencing cassette designed to silence seed lipoxygenases, β-amyrin synthases, oxidosqualene cyclases, and fatty acid desaturases linked in a head to head configuration to the ALS selectable marker recombinant DNA fragment described in Example 3C above. The nucleotide sequence of recombinant DNA fragment PHP19962A is shown in SEQ ID NO:78. Recombinant DNA fragment PHP19962A contains in 5′ to 3′ orientation:
a) the complementary strand of the ALS selectable marker recombinant DNA fragment of Example 3C above;
b) about 2088 nucleotides of the Kti3 promoter;
c) 74-nucleotide synthetic sequence,
d) a unique Not I restriction endonuclease site containing an approximately 3500 nucleotide fragment comprising about 610 nucleotides from the soybean FAD2-1 gene, about 1880 nucleotides from the soybean LOX3 and LOX2 genes, followed by about 570 nucleotides from a soybean β-amyrin synthase gene and about 450 nucleotides from a soybean oxidosqualene cyclase gene;
e) an inverted repeat of the nucleotides in c), and
f) about 202 nucleotides of the Kti3 transcription terminator.

The nucleotide sequence of the approximately 3500 nucleotide fragment is shown in SEQ ID NO:79. The 3500 nucleotide fragment comprising about 610 nucleotides from the FAD2-1 gene, about 1880 nucleotides from the LOX3 and LOX2 genes, followed by about 570 nucleotides from a β-amyrin synthase gene and about 450 nucleotides from an oxidosqualene cyclase gene was constructed by PCR amplification as follows.

An approximately 0.6 kb DNA fragment was obtained by PCR amplification using primers BM31 (the nucleotide sequence of which is shown in SEQ ID NO:80) and BM32 (the nucleotide sequence of which is shown in SEQ ID NO:81) with a DNA fragment comprising recombinant DNA fragment KS136 as template.

BM31:
5′-GCGGCCGCTGAGTGATTGCTCACGAGTGTG-3′
BM32:
5′-TATAAAATGCTCCAGGAATTTTAATCTCTGTCCATAGTTG-3′

An approximately 2.9 kb DNA fragment was obtained by PCR amplification using primers BM33 (the nucleotide sequence of which is shown in SEQ ID NO:82) and BM34 (the nucleotide sequence of which is shown in SEQ ID NO:83) using recombinant DNA fragment PHP19112A as template.

BM33:
5′-CAACTATGGACAGAGATTAAAATTCCTGGAGCATTTTATATC-3′
BM34:
5′-GCGGCCGCCAACAATTTAGAAGAGGCTCGG-3′

The 0.6 kb and the 2.9 kb fragments were mixed and used as template for PCR amplification with primers BM31 (the nucleotide sequence of which is shown in SEQ ID NO:80) and BM34 (the nucleotide sequence of which is shown in SEQ ID NO:83) to yield an approximately 3500 bp fragment. The 3500 bp fragment was cloned into the commercially available plasmid pCR2.1 using the TOPO TA Cloning Kit. After digestion with Not I the 3500 bp fragment was digested with Not I and ligated into the Not I site of plasmid pKS210, described in Example 3F above.

For use in plant transformation experiments the 9997 bp recombinant DNA fragment PHP19962A was removed from its cloning plasmid using restriction endonuclease Asc I and was separated from the remaining plasmid DNA by agarose gel electrophoresis.

### Example 4

### Transformation of Somatic Soybean (Glycine max) Embryo Cultures and Regeneration of Soybean Plants

Soybean embryogenic suspension cultures were transformed by the method of particle gun bombardment using procedures known in the art (Klein et al. (1987) Nature (London) 327:70-73; U.S. Pat. No. 4,945,050; Hazel, et al. (1998) Plant Cell. Rep. 17:765-772; Samoylov, et al. (1998) In Vitro Cell Dev. Biol.-Plant 34:8-13). In particle gun bombardment procedures it is possible to use purified 1) entire plasmid DNA or, 2) DNA fragments containing only the recombinant DNA expression cassette(s) of interest, such as those set forth in Examples 3A-L above.

In the Examples the follow, the recombinant DNA fragments were isolated from the entire plasmid by Asc I digestion and gel electrophoresis before being used for bombardment. For every eight bombardment transformations, 30 μl of solution were prepared with 3 mg of 0.6 μm gold particles and 1 to 90 picograms (pg) of DNA fragment per base pair of DNA fragment.

In the Examples that follow soybean transformation experiments were carried out using one or two recombinant DNA fragments. In most of the transformation experiments (Examples 7B, 8A-C, 9, 11, and 12), all the recombinant DNA fragments used for suppression of gene expression were in the same recombinant DNA fragment as the selectable marker gene. In some of the experiments, such as those described in Examples 6A and B, the recombinant DNA fragment used to suppress the seed lipoxygenases was on a separate recombinant DNA fragment from the selectable marker gene. In some of the other transformation experiments, such as those disclosed in Examples 7A and 10, the recombinant DNA fragment used to suppress expression of FAD2-1 was in a separate recombinant DNA fragment. In the instances where two separate recombinant DNA fragments were used, as in Examples 6A, 6B, 7A, and 10, both recombinant DNA fragments were co-precipitated onto gold particles.

Stock tissue for these transformation experiments were obtained by initiation from soybean immature seeds. Secondary embryos were excised from explants after 6 to 8 weeks on culture initiation medium. The initiation medium was an agar-solidified modified MS (Murashige and Skoog (1962) Physiol. Plant 15:473-497) medium supplemented with vitamins, 2,4-D and glucose. Secondary embryos were placed in flasks in liquid culture maintenance medium and maintained for 7-9 days on a gyratory shaker at 26±2° C. under ˜80 μEm-2s-1 light intensity. The culture maintenance medium was a modified MS medium supplemented with vitamins, 2,4-D, sucrose and asparagine. Prior to bombardment, clumps of tissue were removed from the flasks and moved to an empty 60×15 mm petri dish for bombardment. Tissue was dried by blotting on Whatman #2 filter paper. Approximately 100-200 mg of tissue corresponding to 10-20 clumps (1-5 mm in size each) were used per plate of bombarded tissue.

After bombardment, tissue from each bombarded plate was divided and placed into two flasks of liquid culture maintenance medium per plate of bombarded tissue. Seven days post bombardment, the liquid medium in each flask was replaced with fresh culture maintenance medium supplemented with 100 ng/mL selective agent (selection medium). For selection of transformed soybean cells the selective agent used was a sulfonylurea (SU) compound with the chemical name, 2-chloro-N-((4-methoxy-6 methy-1,3,5-triazine-2-yl)aminocarbonyl)benzenesulfonamide (common names: DPX-W4189 and chlorsulfuron). Chlorsulfuron is the active ingredient in the DuPont sulfonylurea herbicide, GLEAN®. The selection medium containing SU was replaced every week for 6-8 weeks. After the 6-8 week selection period, islands of green, transformed tissue were observed growing from untransformed, necrotic embryogenic clusters. These putative transgenic events were isolated and kept in media with SU at 100 ng/ml for another 2-6 weeks with media changes every 1-2 weeks to generate new, clonally propagated, transformed embryogenic suspension cultures. Embryos spent a total of around 8-12 weeks in SU. Suspension cultures were subcultured and maintained as clusters of immature embryos and also regenerated into whole plants by maturation and germination of individual somatic embryos.

### Example 5

### Lipoxygenase Assay of Transformed Soybean Somatic Embryos and Seeds

All the transgenic plants prepared in this application contain a recombinant DNA fragment designed to suppress soybean seed lipoxygenases. Lipoxygenase (also referred to as LOX) is a dioxygenase that catalyzes, as a primary reaction, the hydroperoxidation, by molecular oxygen, of linoleic acid (18:2) and any other polyunsaturated lipids that contain a cis, cis- 1,4-pentadiene moiety. The recombinant DNA fragments designed to suppress seed lipoxygenases are described in Example 3 above and comprise either a portion of the LOX3 gene, or a portion of the LOX3 gene and a portion of the LOX2 gene. Transgenic plants prepared with any of these constructs and having non-detectable levels of LOX1 activity will be assumed as having all seed lipoxygenases suppressed which is also referred to as being LOX null. Assays were developed to detect LOX1 activity in extracts from soybean somatic embryos, soybean seed chips, and bulk seed. The assays were developed to either measure each sample individually or automatically using a microtiter plate.

Preparation of Soybean Somatic Embryo Extract

Three-week-old somatic soybean embryos were individually ground in 500 μL of 2 mM sodium taurodeoxycholate in a microtiter plate (96 deep-well microtiter plates with a 1.2-2 mL working volume per well) using one 4 mm or 5/32″ steel grinding ball per embryo. The embryos were ground with two 30-45 second cycles at 1500 strokes/min using a Geno/Grinder™ (SPEX CertiPrep, Metuchen, N.J.). The microtiter plates were then centrifuged using a Sorvall Super T21 centrifuge at 500 to 700 rpm for 5 min to remove cellular debris.

Preparation of Soybean Seed Chip Extract

Enzyme extract for lipoxygenase assay from soybean seed chips was prepared as follows. Small soybean seed chips (not more than 5 mm in diameter and as uniform in size as possible), were taken opposite the hypocotyl, placed into the wells of a flat-bottom microtiter plate, and 200 μL of water (sterile-filtered double distilled, and deionized) was added to each well. The microtiter plate was then left on the bench, at room temperature, for three minutes to allow the LOX enzymes to “leach” out of the chips and into solution. The chips were then removed and the extract solution was transferred to a microfuge tube. Any particulates present were removed by centrifuging the enzyme extract for 2-4 minutes at top speed in a micro-centrifuge.

Preparation of Soybean Bulk Seed Extract

The LOX1 enzyme assay was also used to screen soybean seeds in bulk, in order to identify putative homozygous plants in segregating populations. The assay on multiple seeds was carried out as follows. Ten seeds from a single plant were placed into a Geno/Grinder™ with a 9/16-inch stainless steel ball being placed on top of the seeds. The seeds were ground using the Geno/Grinder™ at 1600 rpm for 30 seconds; additional 30-second grindings of the seeds were done until the seeds were pulverized to a homogeneous powder. Ten to 25 mg of pulverized soybean powder was transferred to a 1.5 mL microfuge tube and the soybean powder was suspended, by vortexing, in 500-1,250 μL sterile filtered ddi H₂O to a concentration of 1 μg/50 μL. The vials containing the samples were then inverted and allowed to sit on the bench at room temperature for approximately 3 minutes. Debris was compacted by centrifugation using a micro-centrifuge at top speed.

Assay for Soybean LOX1

Lipoxygenase activity was determined using a spectrophotometric assay where sodium linoleate is hydroperoxidated increasing the 234 nm absorbance of the sample. When measuring LOX1 activity in soybeans (Glycine max cv. Jack) the absorbance at 234 nm increases in 1-3 minutes to about 0.5 or 0.6 OD234nm min-1.

Sodium linoleate substrate was prepared from linoleic acid as follows. Seventy mg of linoleic acid and 70 mg of Tween 20 were weighed out into a 50 mL tube and homogenized in 4 mL sterile filtered double deionized (ddi) H2O. About 0.55 mL of 0.5 N NaOH was added in order to obtain a clear solution. Sterile filtered double distilled H2O was added to bring the solution up to 25 mL total volume. The solution was divided in 2 mL aliquots which were stored at −20° C. under Nitrogen gas. The final stock concentration of sodium linoleate was 10 mM.

To measure lipoxygenase activity in soybean somatic embryos 10 μL of the extract was decanted from each well and transferred to a 96-well standard UV grade microtiter plate suitable for a microtiter plate reader. To each well 100 μL of 0.2 mM sodium linoleate (18:2) in 0.1 M sodium borate, pH 9.0 was added and the increase in absorbance at 234nm was monitored for 3-5 minutes using a microtiter plate reader SpectraMax 190 (Molecular Devices Corp., Sunnyvale, Calif.).

To measure lipoxygenase activity for individual seed samples a mixture of 0.5 mL of 0.2 M Na+ borate, pH 9.0; 0.38 mL of sterile filtered ddi H2O; 0.02 mL of 10 mM sodium linoleate was prepared and used as a blank to zero the spectrophotometer at 234 nm. After the addition of 0.1 mL of soybean seed chip extract (prepared as described above) the absorbance at 234 nm was monitored for 1-3 minutes. The results were compared with the rates of activity obtained for lipoxygenase 1 in soybean (Glycine max cv. Jack) that is 0.5-0.6 OD234 nm min-1.

To measure lipoxygenase activity in soybean bulk seed extracts, 5 μL of the extract from each vial was transferred into a UV grade microtiter plate, 100 μL of 0.2 mM (18:2) sodium linoleate was added, and the increase in absorbance at 234 nm was followed on the microtiter plate reader for 3-5 minutes.

The assay described in this Example was specific for the detection of LOX1. No lipoxygenase activity was observed when this assay was performed on seeds of a soybean mutant known to lack LOX1, and which contains lipoxygenase isozymes LOX2 and LOX3. In contrast, lipoxygenase activity was observed when this assay was performed on seeds of soybean mutants known to contain LOX1 and lack either LOX2 or LOX3. Thus, the somatic embryo LOX1 assay provides a useful test for selection of transformation events likely to yield LOX1 null seeds.

Assay for LOX1, LOX2 and LOX3 Protein

None of the recombinant DNA fragments designed to suppress soybean seed lipoxygenases contained more than 50 contiguous nucleotides from the LOX1 gene. Therefore, it was expected that seeds that lacked LOX1 enzyme activity would also lack LOX2 and LOX3 activities, as one or both of these were present in the recombinant DNA fragments. To assay for all three lipoxygenase proteins at the same time, SDS-polyacrylamide gel electrophoresis of crude protein extracts of soybean seeds was used, essentially as described by Kitamura (1984) Agric. Biol. Chem. 48, 2339-2346.

### Example 6

### Suppression of Activity of Seed Lipoxygenases in Soybean Somatic Embryos and Seeds

The ability to decrease lipoxygenase expression in the seeds of transgenic soybean plants was tested by transforming soybean embryogenic suspension cultures, regenerating fertile transformed plants, and measuring the levels of lipoxygenase in seeds. Two different approaches were taken to reduce the levels of soybean seed lipoxygenases. The gene expression silencing cassette contained 1) nucleotides only from the gene encoding LOX3, or 2) nucleotides from the gene encoding LOX3 and the gene encoding LOX2. The embryogenic suspension cultures were transformed with either recombinant DNA fragment 1025 or recombinant DNA fragment 1028, the construction of which is described in Example 3A and 3B, respectively, each in combination with a DNA fragment carrying the ALS selectable marker gene, which is described in Example 3C. The nucleotides that form the stem-loop structure in recombinant DNA fragment 1025 correspond to a portion of the LOX3 gene while in recombinant DNA fragment 1028 these nucleotides correspond to a portion of the LOX3 gene and a portion of the LOX2 gene. The results obtained from these two transformations follow.

Soybean Embryos Transformed with Recombinant DNA Fragment 1025

Recombinant DNA fragment 1025 was prepared to test whether nucleotides encoding a portion of LOX 3 were capable of suppressing all three soybean seed lipoxygenases. The nucleotide sequence of recombinant DNA fragment 1025 is shown in SEQ ID NO:20. In a soybean transformation experiment using recombinant DNA fragment 1025, thirty eight independently transformed embryogenic suspension cultures found to be resistant to sulfonylurea herbicide were obtained. Five somatic embryos resulting from each of the original 38 suspension cultures were tested for LOX1 activity. Seventeen of the original 38 suspension cultures produced two or more somatic embryos that showed no LOX1 activity. These 17 transformation events are then considered to be LOX1 nulls. Of these 17 events, 13 produced seeds and 9 of the 13 (70%) produced seeds with no detectable LOX1 activity. In contrast, only ten percent of the transformation events that produced less than two of five LOX1 null somatic embryos, also produced LOX1 null seeds. Thus, recombinant DNA fragment 1025 provides a useful system for suppressing seed lipoxygenase activity.

Soybean Embryos Transformed with Recombinant DNA Fragment 1028

Recombinant DNA fragment 1028 was constructed to provide additional sequence similarity to the LOX1 and LOX2 genes in order to more efficiently suppress expression of all three-soybean seed lipoxygenase genes. Recombinant DNA fragment 1028 comprises nucleotides from a portion of the soybean LOX3 gene and a portion of the soybean LOX2 gene. The nucleotide sequence of recombinant DNA fragment 1028 is shown in SEQ ID NO:23. In a soybean transformation experiment using recombinant DNA fragment 1028 one hundred and six independently transformed embryogenic suspension cultures found to be resistant to sulfonylurea herbicide were obtained. These were called individual transformation events. Five somatic embryos resulting from each of the original 106 suspension cultures were tested for LOX1 activity. Sixty nine of the original 106 suspension cultures produced two or more somatic embryos that showed no LOX1 activity. These 69 transformation events were then considered to be LOX1 nulls. The 69 somatic embryos were also assayed by Southern blot analysis to determine the amount and complexity of recombinant DNA insertions. Eight events that contained the simplest insertions of the transforming DNA fragments were selected for regeneration into plants. Five of these eight events produced seeds and all five produced LOX1 null seeds.

The two recombinant DNA fragments, 1025 and 1028, used to suppress soybean seed lipoxygenases contained no more than 50 contiguous nucleotides identical to the LOX1 gene sequence. Recombinant DNA fragment 1025 contained more than 500 contiguous nucleotides identical to the LOX3 gene and recombinant DNA fragment 1028 contained more than 500 nucleotides identical to the LOX3 and the LOX2 genes because fragments from these genes were present in the recombinant DNA fragments. Therefore, it was expected that seeds that lacked LOX1 enzyme activity would also lack LOX2 and LOX3 activities. To assay for all three lipoxygenase proteins at the same time, SDS-polyacrylamide gel electrophoresis was used as described in Example 5. In every case tested, transgenic seeds lacking LOX1 enzyme activity contained no detectable LOX1, LOX2 or LOX3 protein. Thus, the LOX1 assay provides a useful test for selection of transformation events likely to yield LOX1, LOX2 and LOX3 null seeds.

A self-pollinated soybean plant that is homozygous for a knockout transgene will produce seeds, all of which are null for the desired transgene. A greater than 10-fold reduction in the specific enzyme activity is expected in the null seeds when compared to seeds from control plants. Therefore, a sample of seeds from a plant homozygous for a LOX knockout transgene is expected to have less than 10% of the enzyme activity of a wild type plant. Plants that are segregating for the knockout transgene will produce about 25% wild type seeds and 50% hemizygotes and 25% homozygotes. In this case, the enzyme activity in seeds containing the knockout transgene is expected to be about 25% of that for seeds from control plants if the knockout transgene is dominant, or 75 percent of that for seeds from control plants if the knockout transgene is recessive. Based upon these expectations, and using the assay for LOX1 activity in bulk T2 seeds described in Example 5, plants that were homozygous for the LOX knockout transgene were identified from experiments where either recombinant DNA fragment 1025 or recombinant DNA fragment 1028 was used to knockout lipoxygenase. Homozygotes were confirmed by doing multiple (>10) single seed assays as described in Example 5 and finding all seeds to be LOX1 null.

### Example 7

### Suppression of Activity Seed Lipoxygenases and of an Enzyme of the Fatty Acid Desaturation Pathway in Seeds of Transformed Soybean

Simultaneous suppression of seed lipoxygenase and of fatty acid desaturase in soybean seeds was accomplished using two different approaches. In the first instance soybean embryogenic suspension cultures were transformed with recombinant DNA fragment 1029, which comprises a seed lipoxygenase silencing cassette and a selectable marker gene, and recombinant DNA fragment KS136, which comprises a fatty acid desaturase seed-specific gene expression silencing cassette. In the second case soybean tissue was transformed with recombinant DNA fragment PHP19853A, which comprises a gene expression silencing cassette designed to silence seed lipoxygenases and FAD2, linked to the ALS selectable marker gene.

Transformation with Recombinant DNA Fragments 1029 and KS136 Recombinant DNA fragments 1029 and KS136 are described in Example 3D and E, respectively. The nucleotide sequence of recombinant DNA fragment 1029 is shown in SEQ ID NO:29 and the nucleotide sequence of recombinant DNA fragment KS136 is shown in SEQ ID NO:30. Co-precipitation of the recombinant DNA fragments onto gold particles and soybean transformation is described in Example 4. In a soybean transformation experiment using recombinant DNA fragments 1029 and KS136 one hundred fifteen independently transformed embryogenic suspension cultures found to be resistant to sulfonylurea herbicide were obtained and 30 of these (26%) produced LOX1 null somatic embryos.

In order to determine whether the fatty acid composition was altered, which would indicate suppression of the fatty acid desaturase gene expression, the relative amounts of the fatty acids, palmitic, stearic, oleic, linoleic and linolenic, in soybean somatic embryos was determined as follows. Fatty acid methyl esters were prepared from single, mature, somatic soybean embryos or soybean seed chips by transesterification. One embryo, or a chip from a seed, was placed in a vial containing 50 μL of trimethylsulfonium hydroxide and incubated for 30 minutes at room temperature while shaking. After the 30 minutes 0.5 mL of hexane was added, the sample was mixed and allowed to settle for 15 to 30 minutes to allow the fatty acids to partition into the hexane phase. Fatty acid methyl esters (5 μL from hexane layer) were injected, separated, and quantified using a Hewlett-Packard 6890 Gas Chromatograph fitted with an Omegawax 320 fused silica capillary column (Supelco Inc., Cat#24152). The oven temperature was programmed to hold at 220° C. for 2.7 minutes, increase to 240° C. at 20° C. per minute, and then hold for an additional 2.3 minutes. Carrier gas was supplied with a Whatman hydrogen generator. Retention times were compared to those for methyl esters of commercially available standards (Nu-Chek Prep, Inc. catalog #U-99-A).

An increase in oleic acid is indicative of suppression of the FAD2-1 gene. Of 115 independently transformed embryogenic suspension cultures that after transformation were insensitive to sulfonylurea herbicide, 44 (38%) produced somatic embryos with increased levels of oleic acid. Of these 44 transformation events 15 events produced somatic embryos that were both LOX1 null and contained high levels of oleic acid. Plants were regenerated and T1 seeds were produced from some of these events. Seeds were tested for suppression of lipoxygenase activity, as described in Example 5. The fatty acid composition was monitored to determine possible increase in oleic acid as an assay for suppression of the FAD2-1 gene. Plants derived from four events produced seeds exhibiting both the LOX1 null phenotype and the high oleic acid phenotype.

Transformation with Recombinant DNA Fragment PHP19853A

Recombinant DNA fragment PHP19853A is described in Example 3F and comprises a gene expression-silencing cassette designed to silence seed lipoxygenases and FAD2-1 linked to the ALS selectable marker recombinant DNA fragment. The nucleotide sequence of recombinant DNA fragment PHP19853A is shown in SEQ ID NO:32. Precipitation of the recombinant DNA fragment onto gold particles and soybean transformation is described in Example 4.

Of 116 soybean independently transformed embryogenic suspension cultures that after transformation using recombinant DNA fragment PHP19853A were insensitive to sulfonylurea herbicide, 25 (22%) produced LOX1 null somatic embryos. Eighteen of these 25 transformation events (72%) also produced embryos with increased levels of oleic acid, indicative that expression of the FAD2-1 gene was also suppressed. Plants regenerated from 7 of the 18 events produced seeds exhibiting both the LOX1 null phenotype and the high oleic acid phenotype.

### Example 8

### Suppression of Activity of Seed Lipoxygenases and of an Enzyme of the Phenylypropanoid Pathway in Seeds of Transformed Soybean

In order to decrease the amount of lipoxygenase and of an enzyme of the phenylpropanoid pathway in soybean seeds soybean embryogenic suspension cultures were transformed with recombinant DNA fragments designed to suppress seed lipoxygenase and either chalcone synthase, isoflavone synthase, or flavanone 3-hydroxylase.

Transformation with Recombinant DNA Fragment PHP19112A Recombinant DNA fragment PHP19112A is described in Example 3G and contains a gene expression-silencing cassette designed to silence expression of seed lipoxygenases (LOX) and chalcone synthase (CHS) linked to the ALS selectable marker recombinant DNA fragment. The nucleotide sequence of recombinant DNA fragment PHP19112A is shown in SEQ ID NO:38. Precipitation of recombinant DNA fragment PHP19112A onto gold particles and transformation into soybean is described in Example 4.

Of 70 soybean independently transformed embryogenic suspension cultures that after transformation using recombinant DNA fragment PHP19112A were insensitive to sulfonylurea herbicide, 34 (49%) produced LOX1 null somatic embryos. Plants that produced seeds were regenerated from 16 transformation events. Nine of the 16 produced LOX1 null seeds. The levels of isoflavones were tested in seeds from the 9 events that produced LOX1 null seeds, as described below. A reduced level of isoflavones is indicative of suppressed expression of chalcone synthase because this enzyme is required for the production of isoflavones. Five of the nine events that produced LOX1 null seeds also produced seeds with reduced levels of isoflavones.

Single Seed Isoflavone Analysis

A single soybean seed was accurately weighed into a vial and a ⅜ inch stainless steel ball was added. Vials were capped and then placed in a Geno/Grinder™ (Spex Certiprep, Metuchen, N.J.) at 1500 strokes/min for 30 sec. To each vial that contained a pulverized seed, 3.5 mL of methanol:water (80:20 v/v) was added and then the vials were placed in a Geno/Grinder™ for 1 min at 1500 strokes/min. For multiseed assays 8 to 10 seeds were placed into a vial and a 9/16 inch stainless steel ball was added. The vial was capped and then placed in a Geno/Grinder™ at 1600 strokes/min for 30 seconds. Approximately 100 mg aliquot of soy flour was accurately weighed into a vial, 3.5 ml of methanol:water (80:20, v:v) was added to each vial containing pulverized seed and then the vials were placed in a Geno/Grinder™ for 1 min at 1500 strokes/min. The vials were then positioned on an end over end mixer (Glas-Col, Terre Haute, Ind.) for 2 hours at room temperature (approximately 22° C.) and then returned to the Geno/Grinder™ for 1 min at 1500 strokes/min. After the addition of 263 μl of 2N NaOH to each vial the vials were positioned on an end-over-end mixer for 10 minutes at room temperature. Eighty eight μl of glacial acetic acid was added to each vial and mixed. Vials were then centrifuged (Sorvall Super T21, Kendro, Newtown, Conn.) for 20 minutes at room temperature at 3500 rpm in a swinging bucket rotor (ST-H750, Sorvall). Supernatant was analyzed by HPLC (model 1100, Agilent, Wilmington, Del.) equipped with an autosampler at 4° C., diode array detector, and a Luna C18(2) column (4.6 mm×50 mm, 3 micron, Phenomenex, Torrence, Calif.) maintained at 30° C. The column was eluted with 90% A and 10% B (A as 0.1% formic acid in water and B as 0.1% formic acid in acetonitrile) for 5 min at 1 ml/min, 10% B to 22% B from 5 to 11 min at 1 ml/min, 22% B from 11 to 12 min at 1 ml/min, 100% B from 12 to 14.5 min at 2 ml/min, 10% B from 14.6 to 16.5 min at 2 ml/min, and 10% B from 16.5 to 17 min at 1 ml/min. The quantitiy of daidzin, glycitin and genistin were calculated by comparison with standard curves prepared from authentic compounds (Indofine Chemical Co., Soverville, N.J.; Fujico Co., Japan) at 262 nm.

Transformation with Recombinant DNA Fragment PHP191 13A

Recombinant DNA fragment PHP19113A is described in Example 3H and contains a gene expression-silencing cassette designed to silence soybean seed lipoxygenases (LOX) and isoflavone synthase (IFS) linked to the ALS selectable marker recombinant DNA fragment. The nucleotide sequence of recombinant DNA fragment PHP19113A is shown in SEQ ID NO:44. Precipitation of recombinant DNA fragment PHP19113A onto gold particles and transformation into soybean is described in Example 4.

Of 70 independently transformed embryogenic suspension cultures that after transformation using recombinant DNA fragment PHP19113A where insensitive to sulfonylurea herbicide, 25 (36%) produced LOX1 null somatic embryos. Plants that produced seeds were regenerated from twenty of the 25 transformation events. Plants from 14 of the 20 events tested produced LOX1 null seeds. The levels of isoflavones were tested in seeds from 10 events that produced LOX1 null seeds, as described above. A reduced level of isoflavones is indicative of suppressed expression of isoflavone synthase because this enzyme is required for the production of isoflavones. Seeds from 6 of the 10 events exhibiting the LOX1 null phenotype also exhibited reduced levels of isoflavones.

Transformation with Recombinant DNA Fragment PHP19027A

Recombinant DNA fragment PHP19027A is described in Example 31 and contains a lipoxygenase (LOX)-flavanone 3-hydroxylase (F3H) gene expression silencing cassette linked to the ALS selectable marker recombinant DNA fragment. The nucleotide sequence of recombinant DNA fragment PHP19027A is shown in SEQ ID NO:49. Precipitation of recombinant DNA fragment PHP19027A onto gold particles and transformation into soybean is described in Example 4.

In a soybean transformation experiment using recombinant DNA fragment PHP19027A, 61 of 201 sulfonylurea herbicide resistant transformation events (30%) produced LOX1 null somatic embryos. Of 30 events that were tested, 15 produced seeds with the LOX1 null phenotype. The fifteen events that produced LOX1 null seeds were further tested for reduction in the level of flavonols, as described below. A reduced level of flavonols is indicative of suppressed expression of flavanone 3-hydroxylase because this enzyme is required for the production of flavonols. Seeds from five of the events exhibiting the LOX1 null phenotype also exhibited reduced levels of flavonols.

To test for reduction in the level of flavonols in transgenic seeds, the level of kaempferol, the most abundant of the flavonols present in soybean seeds, was determined as follows. Eight to ten seeds were placed into a vial and a 9/16 inch stainless steel ball was added. The vial was capped and then placed in a Geno/Grinder™ Model 2000 (SPEX Certiprep, Metuchen, N.J.) at 1600 strokes/min for 30 seconds. About 100 mg ground soybean was accurately weighed into a beater vial and a ¼ inch stainless steel bead was added along with 1 mL of 60% acetonitrile. The mixture was agitated on a Geno/Grinder™ for 1 minute with the machine set at 1500 strokes per minute and then placed on an end-over-end mixer (Glas-Col, Terre Haute, Ind.) for 1 hour. The vial was then placed in the Geno/Grinder™ for 1 minute with the machine set at 1500 strokes per minute and then centrifuged at 12,000 rpm for 5 minutes. The supernatant was then transferred to a 13×100 mm Pyrex tube fitted with a Teflon cap. One hundred μL of 10 mg/mL aqueous ascorbic acid was added to the extract and the solutions were mixed. Then, 120 μL of 12 N hydrochloric acid was added and the solutions were mixed. Tubes were placed in a heating block at 80° C. for 1 hour. After allowing the tube to cool to room temperature, the volume was measured and the tube was centrifuged at 3500 rpm for 10 minutes. The supernatant was placed in an HPLC vial.

Kaempferol standards were prepared at the following concentrations 0.1, 0.25, 0.5, 1.0 and 2.0 PPM in 60% acetonitrile with 1 mg/mL ascorbic acid. Both samples and standards were analyzed by liquid chromatography/mass spectrometry (LC/MS) according to the following protocol. LC/MS was performed using a Waters (Milford, Mass.) 2690 Alliance HPLC interfaced with a ThermoQuest Finnigan (San Jose, Calif.) LCQ mass spectrometer. Samples were maintained at 20° C. prior to injection. A 10 μL sample was injected onto a Phenomenex (Torrance, Calif.) Luna C18 column (3 1, 4.6 mm×75 mm) maintained at 40° C. Compounds were eluted from the column at a flow rate of 0.8 mL/minute with 90% solvent A (0.1 % formic acid in water) and 10% solvent B (0.1% formic acid in acetonitrile), followed by a linear gradient from 10% B to 20% B from 0 to 0.5 minutes then held at 20% B from 0.5 to 6 minutes, followed by a linear gradient from 20% B to 50% B from 6 to 8 minutes, then 50% B to 95% B from 10 to 12 minutes and then 90% A and 10% B from 12 to 17 minutes. The solvent flow was split post-column with 0.3 mL/minutes diverted to the mass spectrometer. The mass spectrometer was equipped with an ESI source set to scan m/z of 200 to 600 in positive ion mode. The capillary temperature 160° C., the sheath gas flow 60-psi, and the auxiliary gas flow 10 psi.

### Example 9

### Suppression of Activity of Seed Lipoxygenases and of a Second Enzyme of the Lipid Oxidation Pathway in Seeds of Transformed Soybean

In order to decrease the amount of lipoxygenase and of a second enzyme of the lipid oxidation pathway in soybean seeds, soybean embryogenic suspension cultures were transformed with recombinant DNA fragments designed to suppress seed lipoxygenase and hydroperoxide lyase. Recombinant DNA fragment PHP19338A is described in Example 3J and contains a lipoxygenase (LOX)-hydroperoxide lyase (HPL) gene expression silencing cassette linked to the ALS selectable marker gene. The nucleotide sequence of recombinant DNA fragment PHP19338A is shown in SEQ ID NO:54. Precipitation of recombinant DNA fragment PHP19338A onto gold particles and transformation into soybean is described in Example 4.

Of 95 independently transformed embryogenic suspension cultures that after transformation using recombinant DNA fragment PHP19338A were insensitive to sulfonylurea herbicide, 47 (49%) produced LOX1 null somatic embryos. Plants that produced seeds were regenerated from 29 transformation events. Eleven of the 29 events produced LOX1 null seeds. The presence of hydroperoxide lyase mRNA was determined by RT-PCR in ten events that produced seeds with the LOX1 null phenotype, as described below. A reduced level of hydroperoxide lyase mRNA indicated suppressed expression of hydroperoxide lyase. Seeds from five of the ten events exhibiting the LOX1 null phenotype also exhibited reduced levels of hydroperoxide lyase mRNA.

Amplification of HPL mRNA using RT-PCR and PCR

Individual soybean seeds were ground using a vial and ball bearing in a Geno/Grinder™ Model 2000 (SPEX CertiPrep) for 30 seconds at 1550 strokes per minute. One ml of TRIzol (Invitrogen) was added to the vial of each powdered seed, and the mixture was placed in the Geno/Grinder™ again for 30 seconds at 1550 strokes per minute. After mixing, RNA was isolated following Invitrogen's protocol. RNA was resuspended in 100 μl of nuclease-free ddH₂O and stored at −80° C.

First-strand synthesis was performed on individual RNA samples using SuperScript First-Strand Synthesis System for RT-PCR (Invitrogen). Each first-strand synthesis reaction consisted of 1.25 μL of resuspended RNA and 2 μL of random hexamer primer mix (50 ng/μL). Other components were added per manufacturer's protocol. The first-strand synthesis reaction was performed in a GeneAmp PCR System 9700 machine (Applied Biosystems). Temperature regime of each reaction was 10 minutes at 25° C., followed by 50 minutes at 42° C., and followed 15 minutes at 70° C. One microliter of RNase H was added to each reaction, and reactions were incubated for 20 minutes at 37° C. Reactions were stored at −20° C.

PCR amplification of the first-strand reactions was carried out using ReadyMix Taq PCR Reaction Mix with MgCl₂ (Sigma). PCR reactions consisted of: 12.5 μL Sigma Taq Mix, 10.5 μL nuclease-free ddH₂O, 1 μL first-strand reaction, and 0.5 μL of sense and antisense primers (each at 100 μmol/μL). The primer sequences were derived from clone sdp4c.pk015.e22 which, as seen in Example 2C, encodes an entire HPL. The nucleotide sequence of the sense primer is shown in SEQ ID NO:84 and the nucleotide sequence of the antisense primer is shown in SEQ ID NO:85.

| | |
|---|---|
| Sense: | 5′-ATCTTGTGTTCATGTTATCGTTCAACG-3′ |
| Antisense: | 5′-GGCTCCTCCGTCTGGGGTCCGTTGG-3′. |

PCR amplification was performed using a GeneAmp PCR System 9700 machine (Applied Biosystems). Temperature cycles for HPL amplification were: two minutes at 94° C.; followed by 35 cycles of 15 seconds at 94° C., 15 seconds at 54° C., and 45 seconds at 72° C.; followed by three minutes at 72° C. Presence or absence of the HPL cDNA was evaluated by agarose gel electrophoresis.

### Example 10

### Suppression of Activity of Seed Lipoxygenases, of a Second Enzyme of the Lipid Oxidation Pathway, and of an Enzyme of the Fatty Acid Desaturation Pathway in Seeds of Transformed Soybean

In order to decrease the amount of lipoxygenase, decrease the amount of a second enzyme of the lipid oxidation pathway, and decrease the amount of fatty acid desaturase produced in soybean seeds, soybean tissue was co-transformed with recombinant DNA fragment PHP19338A in combination with recombinant DNA fragment KS136. Recombinant DNA fragment PHP19338A is described in Example 3J and contains a lipoxygenase (LOX)-HPL gene expression silencing cassette linked to the ALS selectable marker gene. Recombinant DNA fragment KS136 is described in Example 3E and contains a fatty acid desaturase seed-specific gene expression silencing cassette. The nucleotide sequence of recombinant DNA fragment PHP19338A is shown in SEQ ID NO:54 and the nucleotide sequence of recombinant DNA fragment KS136 is shown in SEQ ID NO:30. Recombinant DNA fragments PHP19338A and KS136 were co-precipitated onto gold particles and transformed into soybean as described in Example 4.

Of 67 independently transformed embryogenic suspension cultures that after transformation using recombinant DNA fragments PHP19338A and KS136 were insensitive to sulfonylurea herbicide, 56 (84%) produced LOX1 null somatic embryos. In order to determine whether the fatty acid composition of the transformed tissue was also altered, which would indicate suppression of fatty acid desaturase gene expression, the relative amounts of the fatty acids, palmitic, stearic, oleic, linoleic and linolenic, in soybean somatic embryos were determined as described in Example 7.

Twenty-two of 45 transformation events (49%) that produced lox null somatic embryos also produced embryos with increased levels of oleic acid, which indicates that expression of the FAD2-1 gene is also suppressed. Plants were regenerated and T1 seeds were produced from some of these events. Seeds were tested for suppression of lipoxygenase activity, as described in Example 5 above. Suppression of the FAD2-1 gene was monitored by determining the relative amounts of the fatty acids, palmitic, stearic, oleic, linoleic and linolenic, in the seeds, as described in Example 7. Five events that produced seeds with both the LOX1 null phenotype and the high oleic acid phenotype were identified. The presence of HPL mRNA was determined by RT-PCR (as described in Example 9) in seeds from three of the five events with both the LOX1 null phenotype and the high oleic acid phenotype. A reduced level of HPL mRNA indicated suppressed expression of HPL. Seeds from all three of the events exhibiting the LOX1 null and the high oleic acid phenotype also exhibited reduced levels of HPL mRNA.

### Example 11

### Suppression of Activity of Seed Lipoxygenases and of Enzymes of the Triterpenoid Pathway in Seeds of Transformed Soybean

In order to decrease the amount of lipoxygenase and decrease the amount of β-amyrin synthase, an oxidosqualene cyclase enzyme of the triterpenoid pathway, in soybean seeds, a DNA fragment containing a lipoxygenase and β-amyrin synthase silencing cassette was constructed. Triterpenoids are composed of the five-carbon isoprenoids. Two molecules of farnesyl pyrophosphate are joined head-to-head to form squalene, a triterpene, in the first dedicated step in the pathway. Squalene is then converted to 2,3-oxidosqualene which, in photosynthetic organisms, may be converted to the 30 carbon, 4-ring structure, cycloartenol or to the 5-ring structure, β-amyrin.

Oxidosqualene cyclases catalyze the cyclization of 2,3-oxidosqualene to form various polycyclic skeletons including one or more of lanosterol, lupeol, cycloartenol, isomultiflorenol, β-amyrin, and α-amyrin. The non-cycloartenol producing oxidosqualene cyclase activities are different, although evolutionarily related, to cycloartenol synthases (Kushiro, T., et al. (1998) Eur. J. Biochem. 256:238-244). β-amyrin synthase catalyzes the cyclization of 2,3-oxidosqualene to β-amyrin and is therefore an example of an oxidosqualene cyclase. The basic β-amyrin ring structure may be modified to give classes of sapogenins, also known as sapogenols. Saponins are glycosylated sapogenins and may play a defense role against pathogens in plant tissues.

Soybean seeds contain several classes of saponin, all of which are formed from one sapogenin ring structure that is modified by hydroxylation and by the addition of different carbohydrate moieties. Total saponin content varies somewhat by soybean cultivar and is in the range of 0.25% of the seed dry weight (Shiraiwa, M., et al. (1991) Agric. Biol. Chem. 55:323-331). The amount of saponin in a sample is proportional to the amount of measured sapogenols. Thus, a relative saponin content may be calculated by measuring the total sapogenols resulting from removing the sugar moieties from the saponin.

Recombinant DNA fragment PHP19104A was used in order to decrease the amount of lipoxygenase and decrease the amount of β-amyrin synthase produced in soybean seeds. Recombinant DNA fragment PHP19104A is described in Example 3K and its nucleotide sequence is shown in SEQ ID NO:67, Recombinant DNA fragment PHP19104A was precipitated onto gold particles and transformed into soybean as described in Example 4.

Of 209 independently transformed embryogenic suspension cultures that after transformation using recombinant DNA fragment PHP19104A were insensitive to sulfonylurea herbicide, 42 (20%) produced LOX1 null somatic embryos. Plants that produced seeds were regenerated from twenty-nine transformation events. Fourteen of the twenty-nine events produced LOX1 null seeds. Nine events that produced seeds with the LOX1 null phenotype have been tested for levels of sapogenols, as described below. A reduced level of sapogenols is indicative of suppressed expression of β-amyrin synthase because this enzyme is required for the production of sapogenols. Seeds from four of the nine events exhibiting the LOX1 null phenotype also exhibited levels of sapogenols reduced by 50 percent or more.

The level of sapogenols present in seeds was determined as follows. Eight to ten seeds were placed into a vial and a 9/16 inch stainless steel ball was added. The vial was capped and then placed in a Model 2000 Geno/Grinder™ at 1600 strokes/min for 30 seconds. About 100 mg ground soybean was accurately weighed into a beater vial and a ¼ inch stainless steel bead was added along with 1 mL of 60% acetonitrile. The mixture was agitated on a Geno/Grinder™ Model 2000 (SPEX Certiprep, Metuchen, N.J.) for 1 minute with the machine set at 1500 strokes per minute and then placed on an end-over-end tumbler for 1 hour. The vial was then placed in the Geno/Grinder™ for 1 minute with the machine set at 1500 strokes per minute and the sediment removed by centrifugation at 12,000 rpm for 4 minutes. The supernatant was then transferred to a 13×100 mm glass test tube fitted with a Teflon® cap. The extraction procedure was repeated once and the supernatants combined into the same 13×100 mm glass test tube. To the tube containing the combined supernatants 0.1 mL of 12N HCl was added. After mixing, the tube was placed into an 80° C. heating block overnight (approximately 16 hours).

After overnight incubation, the tube was removed from the heating block and allowed to cool to room temperature. Next, 5 mL of 12.5% methanol in acetonitrile, 100 μL DMSO and 1.5 mL of methanol was added and the solution was mixed. The volume was measured and recorded. Sediment was removed by centrifuging the tubes for 10 minutes at 3500 rpm at 20° C. and an aliquot of the supernatant was placed into an HPLC vial to analyze the soyasapogenols using liquid chromatography/mass spectrometry (LC/MS).

LC/MS was performed using a Waters TM (Waters Corp., Milford, Mass.) 2690 Alliance HPLC interfaced with a ThermoFinnigan (San Jose, Calif.) LCQ™ mass spectrometer. Samples were maintained at 20° C. prior to injection. A 10 μL sample was injected onto a Phenomenex® (Torrance, Calif.) Luna™ C18(2) column (3 μm, 4.6 mm×50 mm), equipped with a guard cartridge of the same material, and maintained at 40° C. Compounds were eluted from the column at a flow rate of 0.8 mL/minute using a solvent gradient. For the first two minutes the eluent was a 50/50 mixture of solvent A (0.1% formic acid in water) and solvent B (0.1% formic acid in acetonitrile). From 2 to 5 minutes the eluent was a linear gradient from 50% solvent B to 100% solvent B. From 5 to 8 minutes the eluent was 100% solvent B, and from 8 to 11 minutes the eluent was a 50/50 mixture of solvent A and solvent B. The mass spectrometer was equipped with an APCI source set to scan m/z of 250 to 550 in positive ion mode. The vaporizer temperature was set to 400° C., the capillary temperature was at 160° C. and the sheath gas flow was at 60 psi. Identification and quantification of soyasapogenol A and B was based on m/z and co-chromatography of authentic standards (Apin Chemicals, LTD, Oxon, UK).

### Example 12

### Suppression of Activity of Seed Lipoxygenases, of Enzymes of the Triterpenoid Pathway, and of an Enzyme of the Fatty Acid Desaturation Pathway in Seeds of Transformed Soybean

In order to decrease the amount of lipoxygenase, of β-amyrin synthase, of an oxidosqualene cyclase enzyme, and of fatty acid desaturase in soybean seeds, transformed plants were prepared with recombinant DNA fragment PHP19962A. Recombinant DNA fragment PHP19962A is described in Example 3L and contains a lipoxygenase (LOX)-β-amyrin synthase (βAM)-fatty acid desaturase (FAD2) gene expression silencing cassette linked to the ALS selectable marker gene. The nucleotide sequence of recombinant DNA fragment PHP19962A is shown in SEQ ID NO:78. Recombinant DNA fragment PHP19962A was precipitated onto gold particles and transformed into soybean as described in Example 4.

Of 95 independently transformed embryogenic suspension cultures that after transformation using recombinant DNA fragment PHP19962A were insensitive to sulfonylurea herbicide, 31 (33%) produced LOX1 null somatic embryos. Eighteen of the 31 (58%) transformation events that produced LOX1 null somatic embryos also produced embryos with increased levels of oleic acid, indicative that expression of the FAD2-1 gene was also suppressed. Seeds were obtained from 14 of the transformation events that produced somatic embryos that were LOX1 null and showed increased oleic acid. Eight of the 14 events produced LOX1 null seeds. Seeds from 4 of the 8 LOX1 null events also were high in oleic acid. Seeds from 2 of the 4 events exhibiting the LOX1 null and high oleic acid phenotype also exhibited levels of sapogenols reduced by 50 percent or more.

### Example 13

### Preparation of Additional Recombinant DNA Fragments for Suppression of Gene Expression in Seeds of Transformed Soybean

Recombinant DNA fragments were prepared and used in transformation of soybean for the simultaneous suppression of seed lipoxygenases (LOX) and fatty acid desaturases FAD2-1 and FAD2-2, and for simultaneous suppression of seed lipoxygenases (LOX) and fatty acid desaturases FAD2-1 and FAD2-2, and fatty acid desaturase FAD3. A description of the construction of the recombinant DNA fragments follows.

A. Recombinant DNA Fragment KSFAD2-Hybrid

Recombinant DNA Fragment KSFAD2-hybrid contains an approximately 890 polynucleotide fragment comprising about 470 nucleotides from the soybean FAD2-2 gene and 420 nucleotides from the soybean FAD2-1 gene. The nucleotide sequence of recombinant DNA fragment KSFAD2-hybrid is shown in SEQ ID NO:88. Recombinant DNA Fragment KSFAD2-hybrid was constructed as follows.

An approximately 0.47 kb DNA fragment comprising a portion of the soybean FAD2-2 gene was obtained by PCR amplification using primers KS1 (the nucleotide sequence of which is shown in SEQ ID NO:89) and KS2 (the nucleotide sequence of which is shown in SEQ ID NO:90) and using genomic DNA purified from leaves of Glycine max cv. Jack as a template.

KS1: 5′- GCGGCCGCCGGTCCTCTCTCTTTCCGTG -3′
KS2: 5′- TAGAGAGAGTAAGTCCTGCAAGTACTCCTG -3′

An approximately 0.42 kb DNA fragment comprising a portion of the soybean FAD2-1 gene was obtained by PCR amplification using primers KS3 (the nucleotide sequence of which is shown in SEQ ID NO:91) and KS4 (the nucleotide sequence of which is shown in SEQ ID NO:92) and using genomic DNA purified from leaves of Glycine max cv. Jack as a template.

KS3: 5′- CAGGAGTACTTGCAGGACTTACTCTCTCTA -3′
KS4: 5′- GCGGCCGGCCCCTTCTCGGATGTTCCTTC -3′

The 0.47 kb fragment comprising a portion of the soybean FAD2-2 gene and the 0.42 kb fragment comprising a portion of the soybean FAD2-1 gene were gel purified using GeneClean (Qbiogene, Irvine, Calif.), mixed, and used as template for PCR amplification with KS1 and KS4 as primers to yield an approximately 890 bp fragment that was cloned into the commercially available plasmid pGEM-T Easy (Promega, Madison, Wis.) to create a plasmid comprising recombinant DNA Fragment KSFAD2-hybrid.

B. Recombinant DNA Fragment PHP21672A

Recombinant DNA fragment PHP21672A contains a gene expression silencing cassette designed to silence expression of seed lipoxygenases (LOX) and both the FAD2-1 and FAD2-2 genes linked in a head to head configuration to the ALS selectable marker recombinant DNA fragment of Example 3C above. The nucleotide sequence of recombinant DNA fragment PHP21672A is shown in SEQ ID NO:93. Recombinant DNA fragment PHP21672A contains in 5′ to 3′ orientation:
a) the complementary strand of the ALS selectable marker recombinant DNA fragment of Example 3C above,
b) about 2088 nucleotides of the Kti3 promoter,
c) a 74-nucleotide synthetic sequence,
d) an approximately 2779 polynucleotide fragment comprising about 470 nucleotides from the soybean FAD2-2 gene, 420 nucleotides from the soybean FAD2-1 gene and, about 1880 nucleotides from the soybean LOX3 and LOX2 genes inserted at a unique Not I restriction endonuclease site,
e) an inverted repeat of the 74-nucleotide synthetic sequence in c), and
f) about 202 nucleotides of the Kti3 transcription terminator.

The sequence of the approximately 2770 polynucleotide fragment is shown in SEQ ID NO:94. The approximately 2770 polynucleotide fragment comprising about 470 nucleotides from the soybean FAD2-2 gene, 420 nucleotides from the soybean FAD2-1 gene and, about 1880 nucleotides from the soybean LOX3 and LOX2 genes was constructed by PCR amplification as follows.

An approximately 0.9 kb DNA fragment, comprising a portion of the soybean FAD2-2 gene and a portion of the soybean FAD2-1 gene, was obtained by PCR amplification using primers BM35 (the nucleotide sequence of which is shown in SEQ ID NO:95) and BM36 (the nucleotide sequence of which is shown in SEQ ID NO:96) and using as template recombinant DNA fragment KSFAD2-hybrid described in A above.

BM35: 5′-GCGGCCGCCGGTCCTCTCTCTTTCCGTG-3′
BM36: 5′-AAATGCTCCAGGAATTCCCTTCTCGGATGTTC-3′

An approximately 1.9 kb DNA fragment, comprising portions of the LOX2 and LOX3 genes, was obtained by PCR amplification using primers BM37 (the nucleotide sequence of which is shown in SEQ ID NO:97) and BM38 (the nucleotide sequence of which is shown in SEQ ID NO:98) and using recombinant DNA fragment 1028 as template. Recombinant DNA fragment 1028 is described in Example 3B, above.

BM37: 5′- CATCCGAGAAGGGAATTCCTGGAGCATTTTATATC -3′
BM38: 5′- GCGGCCGCCCTCTGAAAGTTAATCCTTCC -3′

The 0.9 kb fragment, comprising a portion of the soybean FAD2-2 gene and a portion of the soybean FAD2-1 gene, and the approximately 1.9 kb fragment, comprising portions of the LOX2 and LOX3 genes, were mixed and used as template for PCR amplification with BM36 and BM38 as primers to yield an approximately 2770 bp fragment that was cloned into the commercially available plasmid pCR2.1 using the TOPO TA Cloning Kit (Invitrogen). After digestion with Not I the approximately 2770 bp fragment having the nucleotide sequence shown in SEQ ID NO:94 was ligated into the Not I site of plasmid pKS210, described in Example 3F above.

For use in plant transformation experiments the 9231 bp recombinant DNA fragment PHP21672A was removed from its cloning plasmid using restriction endonuclease Asc I and was separated from the remaining plasmid DNA by agarose gel electrophoresis.

C. Recombinant DNA Fragment PHP21676A

Recombinant DNA fragment PHP21676A contains a gene expression silencing cassette designed to silence expression of seed lipoxygenases (LOX), the FAD2-1 and FAD2-2 genes, and the FAD3 gene, linked in a head to head configuration to the ALS selectable marker recombinant DNA fragment of Example 3C above. The nucleotide sequence of recombinant DNA fragment PHP21676A is shown in SEQ ID NO:99. Recombinant DNA fragment PHP21676A contains in 5′ to 3′ orientation:
a) the complementary strand of the ALS selectable marker recombinant DNA fragment of Example 3C above,
b) about 2088 nucleotides of the Kti3 promoter,
c) a 74-nucleotide synthetic sequence,
d) an approximately 3414 polynucleotide fragment comprising about 470 nucleotides from the soybean FAD2-2 gene, 420 nucleotides from the soybean FAD2-1 gene, 643 nucleotides from the soybean FAD3 gene and about 1880 nucleotides from the soybean LOX3 and LOX2 genes inserted at a unique Not I restriction endonuclease site,
e) an inverted repeat of the 74-nucleotide synthetic sequence in c), and
f) about 202 nucleotides of the Kti3 transcription terminator.

The sequence of the approximately 3414 polynucleotide fragment is shown in SEQ ID NO:100. The approximately 3414 polynucleotide fragment comprising about 470 nucleotides from the soybean FAD2-2 gene, about 420 nucleotides from the soybean FAD2-1 gene, about 643 nucleotides from the soybean FAD3 gene, and about 1880 nucleotides from the soybean LOX3 and LOX2 genes was constructed by PCR amplification as follows.

An approximately 0.9 kb DNA fragment, comprising a portion of the soybean FAD2-2 gene and a portion of the soybean FAD2-1 gene, was obtained by PCR amplification using primers BM35 (the nucleotide sequence of which is shown in SEQ ID NO:95) and BM39 (the nucleotide sequence of which is shown in SEQ ID NO:101) and using as template recombinant DNA fragment KSFAD2-hybrid described in A above.

BM35: 5′-GCGGCCGCCGGTCCTCTCTCTTTCCGTG-3′
BM39: 5′-TAAACGGTGGAGGAGCCCTTCTCGGATGTTC-3′

An approximately 0.65 kb DNA fragment, comprising a portion of a FAD3 gene, was obtained by PCR amplification using primers BM40 (the nucleotide sequence of which is shown in SEQ ID NO:102) and BM41 (the nucleotide sequence of which is shown in SEQ ID NO:103) and using plasmid pXF1 as template. Plasmid pXF1 comprises a polynucleotide encoding a soybean delta-15 desaturase (FAD3) and is described in U.S. Pat. No. 5,952,544 issued on Sep. 14, 1999. Plasmid pXF1 was deposited with the American Type Culture Collection (ATCC) of Rockville, Md. on Dec. 3, 1991 under the provisions of the Budapest Treaty, and bears Accession Number ATCC 68874.

BM40: 5′- GAACATCCGAGAAGGGCTCCTCCACCGTTTAAG -3′
BM41: 5′- GCGGCCGCCCATAGAGCTTGAGCACTAG -3′

The approximately 0.9 kb fragment, comprising a portion of the soybean FAD2-2 gene and a portion of the soybean FAD2-1 gene, and the approximately 0.65 kb fragment, comprising a portion of a FAD3 gene, were mixed and used as template for a PCR amplification with BM35 and BM41 as primers to yield an approximately 1533 bp fragment that was cloned into the commercially available plasmid pCR2.1 using the TOPO TA Cloning Kit (Invitrogen) to form plasmid Taste24/pC R-TO PO.

An approximately 1.5 kb DNA fragment, comprising a portion of the soybean FAD2-2 gene, a portion of the soybean FAD2-1 gene, and a portion of the soybean FAD3 gene, was obtained by PCR amplification using primers BM35 (the nucleotide sequence of which is shown in SEQ ID NO:95) and BM42 (the nucleotide sequence of which is shown in SEQ ID NO:104) and using plasmid Taste24/pCR-TOPO as a template.

BM35: 5′-GCGGCCGCCGGTCCTCTCTCTTTCCGTG-3′
BM42: 5′-TAAAATGCTCCAGGAATTCCATAGAGCTTGAGCAC-3′

An approximately 1.9 kb DNA fragment, comprising portions of the LOX2 and LOX3 genes, was obtained by PCR amplification using primers BM38 (the nucleotide sequence of which is shown in SEQ ID NO:98) and BM43 (the nucleotide sequence of which is shown in SEQ ID NO:105) and using recombinant DNA fragment 1028 as template. Recombinant DNA fragment 1028 is described in Example 3B, above.

BM38: 5′- GCGGCCGCCCTCTGAAAGTTAATCCTTCC-3′
BM43: 5′- GCTCAAGCTCTATGGAATTCCTGGAGCATTTTATATC-3′

The approximately 1.5 kb fragment, comprising a portion of the FAD2-2 gene, a portion of the FAD2-1 gene, and a portion of the FAD3 gene, was mixed with the approximately 1.9 kb fragment, comprising portions of the LOX2 and LOX3 genes, and used as template for a PCR amplification with BM35 and BM38 as primers to yield an approximately 3414 bp fragment that was cloned into the commercially available plasmid pCR2.1 using the TOPO TA Cloning Kit (Invitrogen).

After digestion with Not I the approximately 3414 bp fragment having the nucleotide sequence shown in SEQ ID NO:100 was ligated into the Not I site of plasmid pKS210, described in Example 3F above.

For use in plant transformation experiments the 9874 bp recombinant DNA fragment PHP21676A was removed from its cloning plasmid using restriction endonuclease Asc I and was separated from the remaining plasmid DNA by agarose gel electrophoresis.

### Example 14

### Suppression of Activity Seed Lipoxygenases and of Enzymes of the Fatty Acid Desaturation Pathway in Seeds of Transformed Soybean

Simultaneous suppression of seed lipoxygenase (LOX) and of fatty acid desaturases in soybean seeds was accomplished using two different approaches than those mentioned in Example 7. In the first instance soybean embryogenic suspension cultures were transformed with recombinant DNA fragment PHP21672A, which comprises a gene expression silencing cassette designed to silence expression of seed lipoxygenases (LOX) and both the FAD2-1 and FAD2-2 genes linked in a head to head configuration to the ALS selectable marker recombinant DNA fragment. In the second case soybean tissue was transformed with recombinant DNA fragment PHP21676A, which comprises a gene expression silencing cassette designed to silence expression of seed lipoxygenases (LOX), of the FAD2-1 and FAD2-2 genes, and the FAD3 gene, linked in a head to head configuration to the ALS selectable marker recombinant DNA fragment.

Transformation of Soybean with Recombinant DNA Fragment PHP21672A Precipitation of the recombinant DNA fragment onto gold particles and soybean transformation is described in Example 4. In a soybean transformation experiment using recombinant DNA fragment PHP21672A 160 independently transformed embryogenic suspension cultures found to be resistant to sulfonylurea herbicide were obtained and 38 of these (24%) produced LOX1 null somatic embryos.

In order to determine whether the fatty acid composition was altered, which would indicate suppression of the fatty acid desaturase gene expression, the relative amounts of the fatty acids, palmitic, stearic, oleic, linoleic and linolenic, in soybean somatic embryos was determined as described in Example 7.

An increase in oleic acid, and a corresponding reduction in linoleic and linolenic acids, is indicative of suppression of the FAD2 genes. Of the 38 transformed embryogenic suspension cultures that produced LOX1 null somatic embryos, 16 produced somatic embryos with increased levels of oleic acid. Plants were regenerated and T1 seeds were produced from 8 of these events. Seeds were tested for suppression of lipoxygenase activity, as described in Example 5 and fatty acid composition was monitored as described in Example 7. Plants derived from 3 events produced seeds exhibiting both the LOX1 null phenotype and the high oleic acid-low polyunsaturated fatty acid phenotype.

Transformation of soybean with Recombinant DNA Fragment PHP21676A Precipitation of the recombinant DNA fragment onto gold particles and soybean transformation is described in Example 4. In a soybean transformation experiment using recombinant DNA fragment PHP21676A 402 independently transformed embryogenic suspension cultures found to be resistant to sulfonylurea herbicide were obtained and 193 of these (48%) produced LOX1 null somatic embryos.

Of the 193 transformed embryogenic suspension cultures that produced LOX1 null somatic embryos, 85 produced somatic embryos with increased levels of oleic acid. Plants were regenerated and T1 seeds were produced from 60 of these events. Seeds were tested for suppression of lipoxygenase activity, as described in Example 5 and fatty acid composition was monitored as described in Example 7. Plants derived from 22 transformation events produced seeds exhibiting both the LOX1 null phenotype and the high oleic acid-low polyunsaturated fatty acid phenotype. About half of these transformation events produced seeds with linolenic acid content below 3% of the total fatty acids. This is a lower linolenic acid level than that obtained from transformations that employed FAD2-1 DNA fragments only.

What is claimed is:

1. A transgenic soybean plant producing seed having reduced activity of seed lipoxygenases when compared to a soybean plant expressing wild type activity of native seed lipoxygenases, said transgenic soybean plant having a nucleic acid fragment from at least a portion of at least one soybean seed lipoxygenase gene, wherein said nucleic acid fragment is capable of suppressing expression of said native seed lipoxygenases and has been introduced into the soybean plant by transformation.

2. A transgenic soybean plant producing seed having:
a) reduced activity of seed lipoxygenases, when compared to a soybean plant expressing wild type activity of native seed lipoxygenases, said transgenic soybean plant having a first nucleic acid fragment from at least a portion of at least one soybean seed lipoxygenase gene, wherein said first nucleic acid fragment is capable of suppressing expression of said native seed lipoxygenases, and
b) reduced activity of a second native enzyme selected from the group consisting of an enzyme of the lipid oxidation pathway, fatty acid desaturation pathway, phenylpropanoid pathway, triterpenoid pathway, and combinations thereof, when compared to a soybean plant expressing wild type activity of said second native enzyme, said transgenic soybean plant having a second nucleic acid fragment from at least a portion of at least one second native enzyme gene, wherein said second nucleic acid fragment is capable of suppressing expression of said native second enzyme,
wherein said first nucleic acid fragment and said second nucleic acid fragment have been introduced into the soybean plant by transformation.

3. The plant of claims 1 or 2 wherein said nucleic acid fragment capable of suppressing expression of native soybean seed lipoxygenases comprises at least a portion of a nucleotide sequence selected from the group consisting of SEQ ID NOS: 1, 2, 3, 4, 5, and 6.

4. The plant of claims 1 or 2 wherein said nucleic acid fragment capable of suppressing expression of native soybean seed lipoxygenases is selected from the group consisting of SEQ ID NOS:20 and 23.

5. The plant of claim 2 wherein said at least one second nucleic acid fragment is selected from the group consisting of fatty acid desaturase, beta-amyrin synthase, oxidosqualene cyclase, isoflavone synthase, chalcone synthase, flavanone 3-hydroxylase, hydroperoxide lyase, and combinations thereof.

6. The plant of claim 2 wherein said enzyme of the lipid oxidation pathway is hydroperoxide lyase.

7. The plant of claim 2 wherein said enzyme of the fatty acid desaturation pathway is selected from the group consisting of fatty acid desaturase 2 and fatty acid desaturase 3.

8. The plant of claim 7 wherein said fatty acid desaturase 2 is selected from the group consisting of fatty acid desaturase 2-1 and fatty acid desaturase 2-2.

9. The plant of claim 2 wherein said enzyme of the phenylpropanoid pathway is selected from the group consisting of isoflavone synthase, chalcone synthase, and flavanone 3-hydroxylase.

10. The plant of claim 2 wherein said enzyme of the triterpenoid pathway is selected from the group consisting of beta-amyrin synthase and oxidosqualene cyclase.

11. A method of suppressing wild type activity of native soybean seed lipoxygenases comprising:
a) transforming plant tissue with a nucleic acid fragment from at least a portion of at least one soybean seed lipoxygenase gene, wherein said nucleic acid fragment is capable of suppressing expression of native soybean seed lipoxygenases,
b) regenerating said plant tissue into a transgenic plant,
c) growing the transgenic plant to produce transgenic seed, and
d) evaluating said transgenic seed for suppression of soybean seed lipoxygenases when compared to seed having wild type activity of native soybean seed lipoxygenases.

12. A method of suppressing wild type activity of native soybean seed lipoxygenases and a second native enzyme selected from the group consisting of an enzyme of the lipid oxidation pathway, the fatty acid desaturation pathway, the phenylpropanoid pathway, the triterpenoid pathway, and combinations thereof, comprising:
a) transforming plant tissue with a first nucleic acid fragment from at least a portion of at least one soybean seed lipoxygenase gene, wherein said nucleic acid fragment is capable of suppressing expression of native soybean seed lipoxygenases, and a second nucleic acid fragment from at least a portion of at least one second enzyme gene, wherein said second nucleic acid fragment is capable of suppressing expression of said second native enzyme,
b) regenerating said plant tissue into a transgenic plant,
c) growing the transgenic plant to produce transgenic seed, and
d) evaluating said transgenic seed for suppression of soybean seed lipoxygenases and suppression of said second native enzyme when compared to seed having wild type activity of soybean seed lipoxygenases and said second native enzyme.

13. The method of claim 12 wherein said second nucleic acid fragment is selected from the group consisting of fatty acid desaturase, beta-amyrin synthase, oxidosqualene cyclase, isoflavone synthase, chalcone synthase, flavanone 3-hydroxylase, hydroperoxide lyase, and combinations thereof.

14. The method of claim 12 wherein said enzyme of the lipid oxidation pathway is hydroperoxide lyase.

15. The method of claim 12 wherein said enzyme of the fatty acid desaturation pathway is selected from the group consisting of fatty acid desaturase 2 and fatty acid desaturase 3.

16. The method of claim 15 wherein said fatty acid desaturase 2 is selected from the group consisting of fatty acid desaturase 2-1 and fatty acid desaturase 2-2.

17. The method of claim 12 wherein said enzyme of the phenylpropanoid pathway is selected from the group consisting of isoflavone synthase, chalcone synthase, and flavanone 3-hydroxylase.

18. The method of claim 12 wherein said enzyme of the triterpenoid pathway is selected from the group consisting of beta-amyrin synthase and oxidosqualene cyclase.

19. Soybean grain from the transgenic plant of claims 1 or 2.

20. Soybean protein product prepared from the soybean grain of claim 19.

21. Soybean oil prepared from the soybean grain of claim 19.

22. Feed prepared from the grain of claim 19.

23. A food prepared from the grain of claim 19.

24. A food prepared with the soybean protein product of claim 20.

25. An industrial product prepared from the grain of claim 19.
